(19) United States
(12) Patent Application Publication
(10) Pub. No.: US 2026/0174502 A1
(43) Pub. Date: Jun. 25, 2026

(54) SYSTEMS AND METHODS FOR GUIDING BONE REMOVAL

(71) Applicant: Stryker Corporation, Portage, MI (US)

(72) Inventors: Christopher ZEH, Parker, CO (US); Brady Lewis WOOLFORD, Mapleton, UT (US); Brian FOUTS, Morgan Hill, CA (US); James FLOM, Redwood City, CA (US); William KAISER, Campbell, CA (US)

(73) Assignee: Stryker Corporation, Portage, MI (US)

(21) Appl. No.: 19/432,058

(22) Filed: Dec. 23, 2025

Related U.S. Application Data
(60) Provisional application No. 63/738,661, filed on Dec. 24, 2024.

(51) Int. Cl. A61B 34/10; A61B 34/00; A61B 90/00

(52) U.S. Cl. CPC A61B 34/10; A61B 34/25; A61B 90/37; A61B 90/39; A61B 2034/105; A61B 2034/107; A61B 2034/252; A61B 2090/374; A61B 2090/3764; A61B 2090/3966

(57) ABSTRACT

Disclosed herein are systems and methods for guiding bone removal. A two-dimensional image that includes bony anatomy and at least one radiopaque object is received. A position and orientation of the radiopaque object(s) relative to a three-dimensional model of the bony anatomy is determined based on the two-dimensional image. An endoscopic image that includes at least one feature associated with the radiopaque object(s) is received. A position and orientation of the feature(s) relative to the endoscopic image is determined. An alignment of the three-dimensional model relative to the endoscopic image is determined based on the position and orientation of the radiopaque object(s) relative to the three-dimensional model and the position and orientation of the feature(s) relative to the endoscopic image. At least a portion of the three-dimensional model is displayed based on the alignment of the three-dimensional model relative to the endoscopic image.

### CROSS-REFERENCE TO RELATED APPLICATIONS

This application claims the benefit of U.S. Provisional Application No. 63/738,661, filed Dec. 24, 2024, the entire contents of which is incorporated herein by reference.

### FIELD

The present disclosure relates generally to orthopedics, and more particularly to methods and systems for guiding bone removal.

### BACKGROUND

Orthopedics is a medical specialty that focuses on the diagnosis, correction, prevention, and treatment of patients with skeletal conditions, including for example conditions or disorders of the bones, joints, muscles, ligaments, tendons, nerves, and skin, which make up the musculoskeletal system. Joint injuries or conditions such as those of the hip joint or other joints can occur from overuse or over-stretching or due to other factors, including genetic factors that may cause deviations from “normal” joint morphology.

Joints are susceptible to a number of different pathologies (e.g., conditions or disorders, which may cause deviation from the normal joint morphology). These pathologies can have both congenital and injury-related origins. In some cases, the pathology can be substantial at the outset. In other cases, the pathology may be minor at the outset but, if left untreated, may worsen over time. More particularly, in many cases an existing pathology may be exacerbated, for example, by the dynamic nature of the joint, the substantial forces imposed on the joint, or a combination thereof. The pathology may, either initially or thereafter, significantly interfere with patient comfort and lifestyle and may require surgical treatment.

Orthopedic surgery commonly involves treating joint pathologies using minimally invasive techniques, such as joint arthroscopy, in which an endoscope is inserted into the joint through a small incision. Procedures performed arthroscopically include debridement of bony pathologies in which portions of bone in a joint that deviate from a “normal” or target morphology are removed. During a debridement procedure, the surgeon uses an endoscopic camera to view the debridement area, but because the resulting endoscopic image has a limited field of view and is somewhat distorted, the surgeon cannot view the entire pathology all at once. As a result, it is challenging for the surgeon to determine exactly where and how much bone should be removed, and whether the shape of the remaining bone has the desired geometry.

Three-dimensional models generated from preoperative images can be used to help a surgeon visualize where and how much bone should be removed from a joint. However, determining which portion of a three-dimensional model corresponds to the limited field of view provided by an endoscopic camera during surgery can be challenging. Furthermore, preoperatively generated three-dimensional models show only the preoperative status of a joint, which does not reflect the changes in joint morphology realized over the course of a bone removal procedure. Accordingly, it can be difficult for a surgeon to gauge whether a sufficient amount of bone has been removed at a given point during surgery.

### SUMMARY

According to various aspects, systems and methods can display a three-dimensional model of bony anatomy of a joint based on a determined position and orientation of the bony anatomy relative to at least one endoscopic image (e.g., a snapshot image or video frame) of the joint to guide a surgeon during a bone removal procedure. The three-dimensional model can be displayed in a position and orientation that aligns with the position and orientation of the bony anatomy in the endoscopic image. Displaying the three-dimensional model in alignment with the endoscopic image can help a surgeon associate the three-dimensional model with the limited view of the joint that the surgeon sees in the endoscopic image. The three-dimensional model may include a representation of planned bone removal that is displayed in association with the endoscopic image to guide the surgeon in bone removal according to the plan. The planned bone removal can be displayed, for example, as an overlay on the bony anatomy in the endoscopic image.

According to an aspect, a method for guiding bone removal includes receiving a two-dimensional image that includes bony anatomy and at least one radiopaque object, such as an object anchored into the bony anatomy, a tool, or a group of small objects or deposits positioned on the bony anatomy. The position and orientation of the radiopaque object(s) relative to a three-dimensional model of the bony anatomy may be determined based on the two-dimensional image. For example, the position and orientation of the three-dimensional model of the bony anatomy relative to the two-dimensional image may be determined by identifying a projection of the three-dimensional model of the bony anatomy that aligns with the two-dimensional image. Similarly, the position and orientation of the radiopaque object(s) relative to the two-dimensional image may be determined by identifying a projection of a three-dimensional model of the radiopaque object(s) that aligns with the radiopaque object(s) in the two-dimensional image. The position and orientation of the radiopaque object(s) relative to the three-dimensional model may then be determined based on the position and orientation of the radiopaque object(s) relative to the two-dimensional image and the position and orientation of the three-dimensional model relative to the two-dimensional image. Alternatively, once the position and orientation of the three-dimensional model relative to the two-dimensional image has been determined, the radiopaque object(s) may be positioned on the surface of the three-dimensional model at locations that would result in the projections of the radiopaque object(s) being aligned with the radiopaque object(s) in the two-dimensional image.

An endoscopic image that includes a feature associated with the radiopaque object(s) may also be received. The feature may be an optically visible portion of the radiopaque object(s), such as the shape of the radiopaque object(s) or a fiducial associated with the radiopaque object(s) (e.g., an ArUco). The feature may also be one or more objects deposited or positioned by a positioning tool, such as an arrangement of pins, beads, gel drops, or pieces of bone wax. The feature may alternatively be one or more depressions left in the bony anatomy by a burring tool. A position and orientation of the feature relative to the endoscopic image may then be determined using image analysis techniques. For example, the known geometry of the feature may be used to determine the position and orientation of the feature that would result in what is seen in the endoscopic image. The known geometry may be a predetermined geometry, such as the predetermined pattern of an ArUco. Based on the determined position and orientation of the feature relative to the endoscopic image, the position and orientation of the radiopaque object(s) relative to the three-dimensional model, and a known relationship between the radiopaque object(s) and the feature, an alignment of the three-dimensional model relative to the endoscopic image may be determined.

At least a portion of the three-dimensional model may be displayed based on the alignment of the three-dimensional model relative to the at least one endoscopic image. The display may include, for example, the three-dimensional model overlaid on the endoscopic image, a side-by-side view of the endoscopic image and three-dimensional model, or a simulated endoscopic image. The display may include an indication of planned bone removal to guide a surgeon during a bone removal procedure.

The alignment between the three-dimensional model and endoscopic images may be updated over time in order to maintain the displayed portion of the three-dimensional model in the correct position and orientation relative to the endoscopic view. The alignment of the three-dimensional model relative to endoscopic images may be updated by tracking the position of the feature as new endoscopic images are received (e.g., by using a motion tracking algorithm that determines image-to-image motion), by using a machine learning model that identifies tissue in endoscopic images, and/or by tracking movement of various aspects of the system (e.g., the bony anatomy and/or an endoscopic imager used to capture the endoscopic images) with inertial measurement units.

Optionally, the three-dimensional model may be updated over time to reflect removal of bone by a surgeon during a bone removal procedure. For example, the position and orientation of a bone removal instrument may be tracked. The bone removal instrument may be tracked by determining the position and orientation of the bone removal instrument in endoscopic images using known characteristics of the bone removal instrument (e.g., a known shape of the bone removal instrument and/or a fiducial marker associated with the bone removal instrument). Movement of the bone removal instrument may be monitored over time by analyzing the endoscopic images and/or by using data from an inertial measurement unit attached to the bone removal instrument. The three-dimensional model may be updated while bone is being removed and the updated model may be displayed to a user, such that changes to the bony anatomy may be visualized by a surgeon in real-time.

An exemplary method for guiding bone removal includes: receiving at least one two-dimensional image comprising bony anatomy and at least one radiopaque object; determining a position and orientation of the at least one radiopaque object relative to a three-dimensional model of the bony anatomy based on the at least one two-dimensional image; receiving at least one endoscopic image comprising at least one feature associated with the at least one radiopaque object; determining a position and orientation of the at least one feature associated with the at least one radiopaque object relative to the at least one endoscopic image; determining an alignment of the three-dimensional model relative to the at least one endoscopic image based on the position and orientation of the at least one radiopaque object relative to the three-dimensional model and the position and orientation of the at least one feature associated with the at least one radiopaque object relative to the at least one endoscopic image; and displaying at least a portion of the three-dimensional model based on the alignment of the three-dimensional model relative to the at least one endoscopic image.

Determining a position and orientation of the at least one radiopaque object relative to the three-dimensional model of the bony anatomy based on the at least one two-dimensional image may include: determining a position and orientation of the three-dimensional model relative to the at least one two-dimensional image; and determining a position and orientation of the at least one radiopaque object relative to the at least one two-dimensional image. Determining a position and orientation of the at least one radiopaque object relative to a three-dimensional model of the bony anatomy based on the at least one two-dimensional image may include: determining a plurality of possible locations of the at least one radiopaque object relative to the three-dimensional model; and selecting a location of the at least one radiopaque object relative to the three-dimensional model based on a position and orientation of a two-dimensional imager used to capture the at least one two-dimensional image relative to the bony anatomy. Displaying the at least a portion of the three-dimensional model may include displaying an overlay of the at least a portion of the three-dimensional model on the at least one endoscopic image. The overlay may be at least partially transparent. Displaying the at least a portion of the three-dimensional model may include displaying the at least a portion of the three-dimensional model separately from the at least one endoscopic image. Displaying the at least a portion of the three-dimensional model may include displaying a simulated endoscopic image that comprises the at least a portion of the three-dimensional model. The at least a portion of the three-dimensional model that is displayed may include an indication of planned bone removal. The indication of planned bone removal may include a heat map.

The at least one feature associated with the at least one radiopaque object may include a fiducial marker. The fiducial marker may include a predetermined pattern. The at least one feature associated with the at least one radiopaque object may include a shape of at least a portion of the at least one radiopaque object. The at least one radiopaque object may include at least one radiopaque object anchored to the bony anatomy or at least one radiopaque material deposited on the bone. A position and orientation of the at least one feature associated with the at least one radiopaque object relative to the three-dimensional model may be determined based on the position and orientation of the at least one radiopaque object relative to the three-dimensional model, and the position and orientation of the at least one feature associated with the at least one radiopaque object relative to the at least one endoscopic image may be determined based on the position and orientation of the at least one feature associated with the at least one radiopaque object relative to the three-dimensional model. The at least one radiopaque object may include a tool for positioning the at least one feature associated with the at least one radiopaque object with respect to the bony anatomy. The at least one radiopaque object may include a radiopaque fiducial attached to a tool for positioning the at least one feature associated with the at least one radiopaque object with respect to the bony anatomy. The at least one feature associated with the at least one radiopaque object may include one or more markings in the bony anatomy made by the tool, bone wax or gel drops deposited by the tool, one or more depressions formed in the bony anatomy by the tool, or one or more pins inserted into the bony anatomy by the tool. The at least one radiopaque object may include a plurality of radiopaque objects, and the at least one feature associated with the at least one radiopaque object may include an optically visible portion of at least one of the plurality of radiopaque objects. The plurality of radiopaque objects may include a plurality of pins. The at least one feature associated with the at least one radiopaque object may not be radiopaque.

The method may include receiving a new endoscopic image comprising the at least one feature associated with the at least one radiopaque object; determining a position and orientation of the at least one feature associated with the at least one radiopaque object relative to the new endoscopic image; and determining an alignment of the three-dimensional model relative to the new endoscopic image based on the position and orientation of the at least one radiopaque object relative to the three-dimensional model and the position and orientation of the at least one feature associated with the at least one radiopaque object relative to the new endoscopic image. The method may include updating the display of the at least a portion of the three-dimensional model based on the alignment of the three-dimensional model relative to the new endoscopic image. The method may include monitoring motion of an endoscopic imager using at least one inertial measurement unit and updating the display of the at least a portion of the three-dimensional model based on the monitored motion of the endoscopic imager. The method may include monitoring motion of an endoscopic imager based on a plurality of endoscopic images and updating the display of the at least a portion of the three-dimensional model based on the monitored motion. The method may include monitoring motion of the bony anatomy using at least one inertial measurement unit and updating the display of the at least a portion of the three-dimensional model based on the monitored motion of the bony anatomy.

The method may include tracking a bone removal instrument while the bone removal instrument removes bone and updating the at least a portion of the three-dimensional model to reflect bone removal based on the tracking of the bone removal instrument. Tracking the bone removal instrument may include analyzing endoscopic images to determine a position and orientation of the bone removal instrument. Analyzing the endoscopic images to determine the position and orientation of the bone removal instrument may include analyzing a fiducial marker associated with the bone removal instrument. Tracking the bone removal instrument may include tracking motion of the bone removal instrument using at least one inertial measurement unit. Tracking the bone removal instrument may include maintaining alignment of the three-dimensional model relative to endoscopic images based on at least one fiducial attached to the bone.

The method may include, while tracking the bone removal instrument, maintaining alignment of the three-dimensional model relative to endoscopic images by: analyzing the endoscopic images to identify at least a portion of the bony anatomy in the endoscopic images; and matching the at least a portion of the bony anatomy in the endoscopic images to a corresponding portion of the three-dimensional model. The method may include, while tracking the bone removal instrument, maintaining alignment of the three-dimensional model relative to endoscopic images by: receiving endoscopic images comprising the bone removal instrument positioned at a location of the at least one feature associated with the at least one radiopaque object; determining a position and orientation of the bone removal instrument relative to the endoscopic images; and determining a position and orientation of the three-dimensional model relative to the endoscopic images based on the position and orientation of the bone removal instrument relative to the endoscopic images and a position and orientation of the at least one feature associated with the at least one radiopaque object relative to the three-dimensional model.

The method may include: monitoring motion of the bony anatomy using a first inertial measurement unit; monitoring motion of an endoscopic imager using a second inertial measurement unit; monitoring motion of a bone removal instrument using a third inertial measurement unit and a fiducial attached to the bone removal instrument; and updating display of the at least a portion of the three-dimensional model based on the monitored motion of the bony anatomy, the monitored motion of the endoscopic imager, and the monitored motion of the bone removal instrument. The method may include: monitoring motion of an endoscopic imager using a first inertial measurement unit; monitoring motion of a bone removal instrument using a second inertial measurement unit and a fiducial attached to the bone removal instrument; and updating display of the at least a portion of the three-dimensional model based on the monitored motion of the endoscopic imager and the monitored motion of the bone removal instrument. The method may include: monitoring motion of an endoscopic imager using at least one inertial measurement unit; monitoring motion of a bone removal instrument using a fiducial attached to the bone removal instrument; and updating display of the at least a portion of the three-dimensional model based on the monitored motion of the endoscopic imager and the monitored motion of the bone removal instrument.

The method may include: while displaying the at least a portion of three-dimensional model, drilling at least one depth guide in the bony anatomy; and removing bone based on the at least one depth guide.

An exemplary method for guiding bone removal includes: receiving at least one two-dimensional image comprising bony anatomy and at least one radiopaque feature of an endoscopic imager; determining a position and orientation of the endoscopic imager relative to a three-dimensional model of the bony anatomy based on the at least one two-dimensional image; determining an alignment of the three-dimensional model relative to at least one endoscopic image captured by the endoscopic imager based on the position and orientation of the endoscopic imager relative to the three-dimensional model and one or more properties of the endoscopic imager; and displaying at least a portion of the three-dimensional model based on the alignment of the three-dimensional model relative to the at least one endoscopic image.

An exemplary system for guiding bone removal includes one or more processors and memory storing one or more programs for execution by the one or more processors, the one or more programs including instructions that, when executed by the one or more processors, cause the system to: receive at least one two-dimensional image comprising bony anatomy and at least one radiopaque object; determine a position and orientation of the at least one radiopaque object relative to a three-dimensional model of the bony anatomy based on the at least one two-dimensional image; receive at least one endoscopic image comprising at least one feature associated with the at least one radiopaque object; determine a position and orientation of the at least one feature associated with the at least one radiopaque object relative to the at least one endoscopic image; determine an alignment of the three-dimensional model relative to the at least one endoscopic image based on the position and orientation of the at least one radiopaque object relative to the three-dimensional model and the position and orientation of the at least one feature associated with the at least one radiopaque object relative to the at least one endoscopic image; and display at least a portion of the three-dimensional model based on the alignment of the three-dimensional model relative to the at least one endoscopic image.

Determining a position and orientation of the at least one radiopaque object relative to the three-dimensional model of the bony anatomy based on the at least one two-dimensional image may include: determining a position and orientation of the three-dimensional model relative to the at least one two-dimensional image; and determining a position and orientation of the at least one radiopaque object relative to the at least one two-dimensional image. Determining a position and orientation of the at least one radiopaque object relative to a three-dimensional model of the bony anatomy based on the at least one two-dimensional image may include: determining a plurality of possible locations of the at least one radiopaque object relative to the three-dimensional model; and selecting a location of the at least one radiopaque object relative to the three-dimensional model based on a position and orientation of a two-dimensional imager used to capture the at least one two-dimensional image relative to the bony anatomy. Displaying the at least a portion of the three-dimensional model may include displaying an overlay of the at least a portion of the three-dimensional model on the at least one endoscopic image. The overlay may be at least partially transparent. Displaying the at least a portion of the three-dimensional model may include displaying the at least a portion of the three-dimensional model separately from the at least one endoscopic image. Displaying the at least a portion of the three-dimensional model may include displaying a simulated endoscopic image that comprises the at least a portion of the three-dimensional model. The at least a portion of the three-dimensional model that is displayed may include an indication of planned bone removal. The indication of planned bone removal may include a heat map.

The at least one feature associated with the at least one radiopaque object may include a fiducial marker. The fiducial marker may include a predetermined pattern. The at least one feature associated with the at least one radiopaque object may include a shape of at least a portion of the at least one radiopaque object. The at least one radiopaque object may include at least one radiopaque object anchored to the bony anatomy or at least one radiopaque material deposited on the bone. A position and orientation of the at least one feature associated with the at least one radiopaque object relative to the three-dimensional model may be determined based on the position and orientation of the at least one radiopaque object relative to the three-dimensional model, and the position and orientation of the at least one feature associated with the at least one radiopaque object relative to the at least one endoscopic image may be determined based on the position and orientation of the at least one feature associated with the at least one radiopaque object relative to the three-dimensional model. The at least one radiopaque object may include a tool for positioning the at least one feature associated with the at least one radiopaque object with respect to the bony anatomy. The at least one radiopaque object may include a radiopaque fiducial attached to a tool for positioning the at least one feature associated with the at least one radiopaque object with respect to the bony anatomy. The at least one feature associated with the at least one radiopaque object may include one or more markings in the bony anatomy made by the tool, bone wax or gel drops deposited by the tool, one or more depressions formed in the bony anatomy by the tool, or one or more pins inserted into the bony anatomy by the tool. The at least one radiopaque object may include a plurality of radiopaque objects, and the at least one feature associated with the at least one radiopaque object may include an optically visible portion of at least one of the plurality of radiopaque objects. The plurality of radiopaque objects may include a plurality of pins. The at least one feature associated with the at least one radiopaque object may not be radiopaque.

The one or more programs may include instructions for receiving a new endoscopic image comprising the at least one feature associated with the at least one radiopaque object; determining a position and orientation of the at least one feature associated with the at least one radiopaque object relative to the new endoscopic image; and determining an alignment of the three-dimensional model relative to the new endoscopic image based on the position and orientation of the at least one radiopaque object relative to the three-dimensional model and the position and orientation of the at least one feature associated with the at least one radiopaque object relative to the new endoscopic image. The one or more programs may include instructions for updating the display of the at least a portion of the three-dimensional model based on the alignment of the three-dimensional model relative to the new endoscopic image. The one or more programs may include instructions for monitoring motion of an endoscopic imager using at least one inertial measurement unit and updating the display of the at least a portion of the three-dimensional model based on the monitored motion of the endoscopic imager. The one or more programs may include instructions for monitoring motion of an endoscopic imager based on a plurality of endoscopic images and updating the display of the at least a portion of the three-dimensional model based on the monitored motion. The one or more programs may include instructions for monitoring motion of the bony anatomy using at least one inertial measurement unit and updating the display of the at least a portion of the three-dimensional model based on the monitored motion of the bony anatomy.

The one or more programs may include instructions for tracking a bone removal instrument while the bone removal instrument removes bone and updating the at least a portion of the three-dimensional model to reflect bone removal based on the tracking of the bone removal instrument. Tracking the bone removal instrument may include analyzing endoscopic images to determine a position and orientation of the bone removal instrument. Analyzing the endoscopic images to determine the position and orientation of the bone removal instrument may include analyzing a fiducial marker associated with the bone removal instrument. Tracking the bone removal instrument may include tracking motion of the bone removal instrument using at least one inertial measurement unit. Tracking the bone removal instrument may include maintaining alignment of the three-dimensional model relative to endoscopic images based on at least one fiducial attached to the bone.

The one or more programs may include instructions for, while tracking the bone removal instrument, maintaining alignment of the three-dimensional model relative to endoscopic images by: analyzing the endoscopic images to identify at least a portion of the bony anatomy in the endoscopic images; and matching the at least a portion of the bony anatomy in the endoscopic images to a corresponding portion of the three-dimensional model. The one or more programs may include instructions for, while tracking the bone removal instrument, maintaining alignment of the three-dimensional model relative to endoscopic images by: receiving endoscopic images comprising the bone removal instrument positioned at a location of the at least one feature associated with the at least one radiopaque object; determining a position and orientation of the bone removal instrument relative to the endoscopic images; and determining a position and orientation of the three-dimensional model relative to the endoscopic images based on the position and orientation of the bone removal instrument relative to the endoscopic images and a position and orientation of the at least one feature associated with the at least one radiopaque object relative to the three-dimensional model.

The one or more programs may include instructions for: monitoring motion of the bony anatomy using a first inertial measurement unit; monitoring motion of an endoscopic imager using a second inertial measurement unit; monitoring motion of a bone removal instrument using a third inertial measurement unit and a fiducial attached to the bone removal instrument; and updating display of the at least a portion of the three-dimensional model based on the monitored motion of the bony anatomy, the monitored motion of the endoscopic imager, and the monitored motion of the bone removal instrument. The one or more programs may include instructions for: monitoring motion of an endoscopic imager using a first inertial measurement unit; monitoring motion of a bone removal instrument using a second inertial measurement unit and a fiducial attached to the bone removal instrument; and updating display of the at least a portion of the three-dimensional model based on the monitored motion of the endoscopic imager and the monitored motion of the bone removal instrument. The one or more programs may include instructions for: monitoring motion of an endoscopic imager using at least one inertial measurement unit; monitoring motion of a bone removal instrument using a fiducial attached to the bone removal instrument; and updating display of the at least a portion of the three-dimensional model based on the monitored motion of the endoscopic imager and the monitored motion of the bone removal instrument.

The one or more programs may include instructions for: while displaying the at least a portion of three-dimensional model, drilling at least one depth guide in the bony anatomy; and removing bone based on the at least one depth guide.

An exemplary system for guiding bone removal includes one or more processors and memory storing one or more programs for execution by the one or more processors, the one or more programs including instructions that, when executed by the one or more processors, cause the system to: receive at least one two-dimensional image comprising bony anatomy and at least one radiopaque feature of an endoscopic imager; determine a position and orientation of the endoscopic imager relative to a three-dimensional model of the bony anatomy based on the at least one two-dimensional image; determine an alignment of the three-dimensional model relative to at least one endoscopic image captured by the endoscopic imager based on the position and orientation of the endoscopic imager relative to the three-dimensional model and one or more properties of the endoscopic imager; and display at least a portion of the three-dimensional model based on the alignment of the three-dimensional model relative to the at least one endoscopic image.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1A is a schematic view of an exemplary surgical suite;

FIG. 1B is a functional block diagram of an endoscopic imaging system;

FIG. 2 illustrates an exemplary method for guiding a surgeon in the removal of bone from a portion of a joint during a surgical procedure;

FIGS. 3A-3C illustrate exemplary two-dimensional images that include radiopaque objects;

FIG. 4 illustrates an exemplary three-dimensional model of bony anatomy;

FIG. 5 illustrates an exemplary method for determining an alignment of a three-dimensional model of bony anatomy relative to a two-dimensional image of the bony anatomy;

FIG. 6 illustrates aspects of an exemplary application of the method of FIG. 5 to a femur;

FIGS. 7A-7D illustrate exemplary endoscopic images that include optical features associated with radiopaque objects;

FIG. 8A illustrates an exemplary visualization of a three-dimensional model of bony anatomy overlaid on an endoscopic image of the bony anatomy;

FIG. 8B illustrates an exemplary visualization of a three-dimensional model of bony anatomy and a representation of a surgical instrument overlaid on an endoscopic image of the bony anatomy;

FIG. 9 illustrates an exemplary side-by-side visualization of an endoscopic image of bony anatomy and a three-dimensional model of the bony anatomy;

FIG. 10 illustrates an exemplary simulated endoscopic image;

FIG. 11 illustrates an exemplary implementation of the method of FIG. 2 that uses a radiopaque tool and a plurality of non-radiopaque objects to determine an alignment between a three-dimensional model and an endoscopic image;

FIG. 12 illustrates an exemplary implementation of the method of FIG. 2 that uses a radiopaque tool and a plurality of depressions to determine an alignment between a three-dimensional model and an endoscopic image;

FIG. 13 illustrates an exemplary implementation of the method of FIG. 2 that uses a plurality of radiopaque objects to determine an alignment between a three-dimensional model and an endoscopic image;

FIG. 14 illustrates exemplary depth guides for guiding bone removal;

FIG. 15 illustrates an exemplary method for guiding a surgeon in the removal of bone from a portion of joint during a surgical procedure;

FIG. 16 illustrates an exemplary computing system;

FIG. 17 illustrates an exemplary graphical user interface displaying an interactive checklist for guiding a surgeon through a surgical procedure;

FIG. 18 illustrates an exemplary graphical user interface displaying an interactive checklist for guiding a surgeon to place bony anatomy in a plurality of positions;

FIG. 19 illustrates an exemplary graphical user interface for guiding a surgeon in touching a tracked surgical instrument to bony anatomy to generate a three-dimensional model;

FIG. 20 illustrates an exemplary process flow for generating a three-dimensional model of bony anatomy from a determined surface of the bony anatomy;

FIG. 21 illustrates an exemplary method for generating a three-dimensional model of bony anatomy;

FIG. 22 illustrates an exemplary side-by-side visualization of an endoscopic image of bony anatomy and a three-dimensional model of the bony anatomy in which the endoscopic image and the three-dimensional model have a different scale;

FIG. 23 illustrates a three-dimensional model of bony anatomy with a plurality of bur pockets around the perimeter of a planned bone removal area;

FIG. 24 illustrates a three-dimensional model of bony anatomy in which bone that has been resected is shown using different coloration;

FIG. 25A illustrates a three-dimensional model of bony anatomy in which a portion of a heat map is removed in an area corresponding to resected bone;

FIG. 25B illustrates a three-dimensional model of bony anatomy in which portion of a heat map is removed in an area corresponding to resected bone and the area is outlined;

FIG. 25C illustrates a three-dimensional model of bony anatomy in which a portion of a heat map is colorized in an area corresponding to bone that has been resected to a target depth; and

FIG. 25D illustrates a three-dimensional model of bony anatomy in which a portion of a heat map changes color to reflect a remaining amount of bone to be resected.

### DETAILED DESCRIPTION

Reference will now be made in detail to implementations of various aspects and variations of the invention, examples of which are illustrated in the accompanying drawings. Various devices, systems, and methods are described herein. Although at least two variations of the devices, systems, and methods are described, other variations may include aspects of the devices, systems, and methods described herein combined in any suitable manner having combinations of all or some of the aspects described. Exemplary aspects will now be described more fully hereinafter with reference to the accompanying drawings; however, they may be embodied in different forms and should not be construed as limited to the examples set forth herein. Rather, these examples are provided so that this disclosure will be thorough and complete, and will fully convey exemplary implementations to those skilled in the art.

According to various aspects, systems and methods according to the principles described herein include displaying a three-dimensional model of bony anatomy of a joint or a portion thereof based on an alignment of the three-dimensional model relative to an endoscopic image of the joint. An image processing system may receive a two-dimensional image that includes bony anatomy and at least one radiopaque object. For example, the at least one radiopaque object may be a radiopaque object with a known geometry that is implanted in the bony anatomy. A position and orientation of the radiopaque object relative to the three-dimensional model may be determined based on the two-dimensional image. The position and orientation of the radiopaque object relative to the two-dimensional image may be determined by manipulating the position and orientation of a three-dimensional model of the radiopaque object until a projection of the model aligns with the two-dimensional image. The three-dimensional model may also be aligned with the two-dimensional image using a similar method in which the position and orientation of the three-dimensional model is iteratively adjusted until a projection of the model into two-dimensional space aligns with the two-dimensional image. The position and orientation of the radiopaque object relative to the three-dimensional model may then be determined using the position and orientation of the radiopaque object relative to the two-dimensional image and the position and orientation of the three-dimensional model relative to the two-dimensional image.

The image processing system may also receive an endoscopic image that includes at least one feature associated with the radiopaque object. Using the example above, the radiopaque object may be an object with a known geometry, and the feature associated with the radiopaque object may be a fiducial marker, such as an ArUco or QR code printed on or attached to the at least one radiopaque object. A position and orientation of the feature relative to the endoscopic image may then be determined (e.g., using image analysis techniques).

An alignment of the three-dimensional model relative to the endoscopic image may be determined based on the position and orientation of the radiopaque object relative to the three-dimensional model and the position and orientation of the feature relative to the endoscopic image. The relationship between the radiopaque object and the feature may be known. A three-dimensional model of the radiopaque object stored in a memory of the image processing system may indicate the position of the feature with respect to the three-dimensional model of the radiopaque object. For example, the three-dimensional model of the radiopaque object stored in a memory of an image processing system may indicate a surface of the radiopaque object a fiducial marker is attached to or printed on. Based on the known relationship between the radiopaque object and the feature, the position and orientation of the radiopaque object relative to the endoscopic image may be determined, and/or the position and orientation of the feature relative to the three-dimensional model may be determined. Based on the position and orientation of the radiopaque object relative to the endoscopic image and the position and orientation of the radiopaque object relative to the three-dimensional model (or based on the position and orientation of the feature relative to the endoscopic image and the position and orientation of the feature relative to the three-dimensional model), the alignment of the three-dimensional model relative to the endoscopic image may be determined.

In some aspects, other types of radiopaque objects and associated features may be used to determine an alignment of the three-dimensional model relative to the endoscopic image. For example, the radiopaque object may be a tool used to deposit or position objects with respect to the bony anatomy, and the associated feature may include a plurality of small objects (e.g., pins, beads, gel drops, or pieces of bone wax). Alternatively, the radiopaque object may be a bone removal tool (e.g., a bur), and the associated feature may be a plurality of depressions (also referred to herein as “bur pockets”) created by the bone removal tool.

At least a portion of the three-dimensional model may be displayed based on the alignment of the three-dimensional model relative to the endoscopic image. For example, the three-dimensional model or a portion thereof may be overlaid on the endoscopic image or displayed side-by-side with the endoscopic image. Alternatively, a simulated endoscopic image may be displayed. The display may optionally include an indication of planned bone removal (e.g., a heat map), which may be overlaid on the at least a portion of the three-dimensional model.

The alignment of the three-dimensional model relative to the endoscopic view may be updated over time to maintain the displayed portion of the three-dimensional model in the correct position and orientation relative to the endoscopic view. The alignment of the three-dimensional model relative to the endoscopic view may be maintained by re-aligning the three-dimensional model with new endoscopic images. For example, the three-dimensional model may be re-aligned with new endoscopic images by tracking the position of the feature as new endoscopic images are received. A motion tracking algorithm may be used to determine image-to-image motion of the feature. In some aspects, the feature may not be visible in the endoscopic view (e.g., if the bone or the endoscopic imager has moved). If the feature is not visible, alignment between the three-dimensional model and endoscopic images may be maintained by analyzing the endoscopic images to identify the bony anatomy pictured in the endoscopic images. For example, a machine learning model may be used to identify tissue in the new endoscopic images, and the identified tissue may be matched with a bone surface of the three-dimensional model. Other machine learning models that do not identify tissue may also be used to maintain alignment. For example, a machine learning model (e.g., a deep learning model) may use similarity metrics to determine changes in what is shown in the endoscopic images (and, thereby, motion of tissue) without identifying the tissue shown in the endoscopic images. Alternatively, or additionally, the alignment may be maintained using one or more sensors, such as one or more inertial measurement units (IMUs). For instance, IMUs may be attached to the endoscopic imager used to capture the endoscopic images and/or the bony anatomy in order to track their movement.

In some examples, the displayed portion of the three-dimensional model may be used to guide a surgeon during a bone removal procedure. For example, the displayed portion of the three-dimensional model may be updated as a surgical procedure progresses in order to reflect the removal of bone. The three-dimensional model may be updated by tracking a bone removal instrument (e.g., a bur, shaver, drill, etc.). The bone removal instrument may be tracked using an endoscopic imager. A fiducial marker (e.g., an ArUco or QR code) may be attached to the bone removal instrument, and endoscopic images that include the fiducial marker may be captured and analyzed to determine the location of the bone removal instrument. Alternatively or in addition, the bone removal instrument may be tracked using an IMU attached to the bone removal instrument. The three-dimensional model may be updated while bone is being removed and the updated model may be displayed to a user, such that changes to the bony anatomy may be visualized by a surgeon in real-time.

Optionally, the displayed portion of the three-dimensional model may be used by a surgeon to drill depth guides in the bony anatomy for guiding bone removal. A depth chart corresponding to the indication of planned bone removal may indicate to a surgeon how deep of a depth guide to drill in various portions of the bony anatomy. Once the surgeon has drilled depth guides based on the depth chart, the surgeon can use the depth guides to determine how much bone to remove in different areas of the bony anatomy. The surgeon may resect bone to a depth consistent with each of the depth guides in the areas surrounding the respective depth guides.

As used herein, “bone removal” encompasses any method of resecting bone, including drilling, sawing, burring, and bone removal using an osteotome. As used herein, the term “endoscopic image” encompasses single snapshot images and one or more video frames.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system memories or registers or other such information storage, transmission, or display devices.

The present disclosure in some aspects also relates to devices or systems for performing the operations herein. The devices or systems may be specially constructed for the required purposes, may comprise a general-purpose computer selectively activated or reconfigured by a computer program stored in the computer, or may include any combination thereof. Computer instructions for performing the operations herein can be stored in any combination of non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMS, EEPROMs, magnetic or optical cards, or any type of media suitable for storing electronic instructions, and each coupled to a computer system bus. One or more instructions for performing the operations herein may be implemented in or executed by one or more Application Specific Integrated Circuits (ASICs), Field Programmable Gate Arrays (FPGAs), Digital Signal Processing units (DSPs), Graphics Processing Units (GPUs), Central Processing Units (CPUs), or any other suitable processing unit. Furthermore, the computers referred to herein may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

Although the following examples often refer to hip joints, hip joint pathologies, and hip joint characteristics and measurements, it is to be understood that the systems, methods, techniques, visualizations, etc., described herein can be used for analyzing and visualizing other joints, including knees, shoulders, elbows, the spine, the ankle, etc.

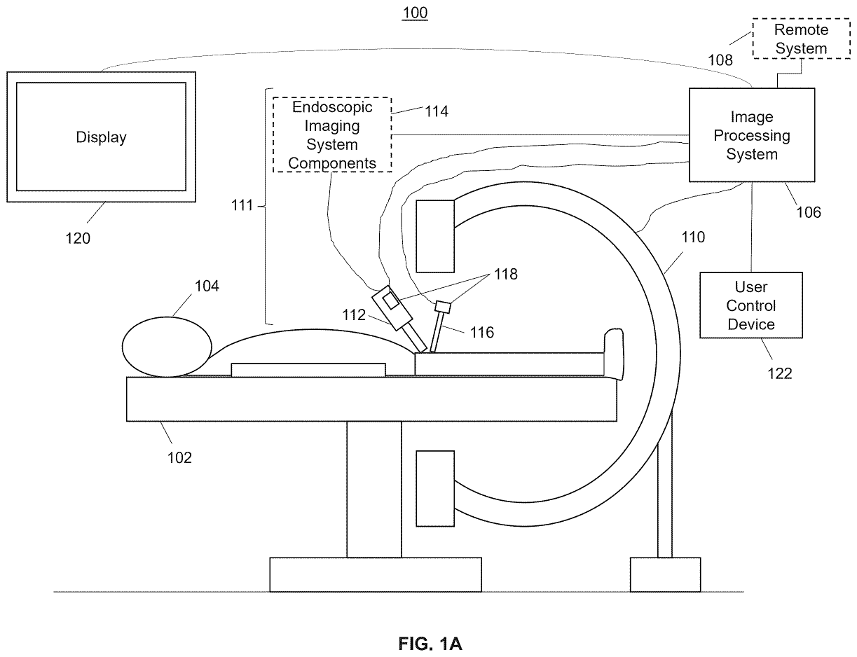

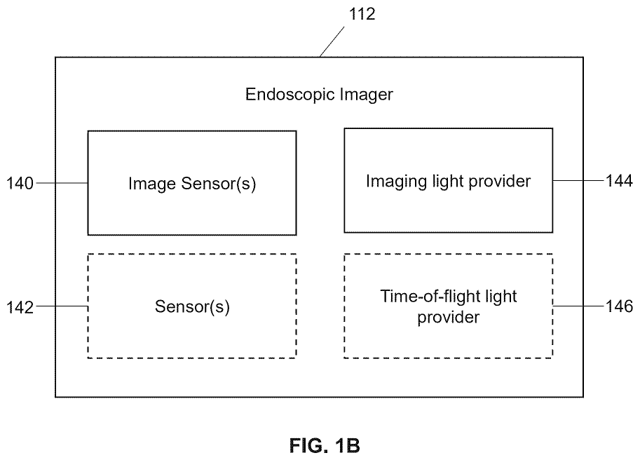

Systems and methods for guiding a surgeon during a surgical procedure on a joint can include displaying at least a portion of a three-dimensional model based on a determined alignment of the three-dimensional model relative to an endoscopic image. FIG. 1A illustrates an exemplary surgical suite incorporating a system 100 configured to display a three-dimensional model based on a determined alignment of the three-dimensional model relative to an endoscopic image. System 100 may include a surgical table 102 on which a patient 104 may be positioned. A two-dimensional imaging system 110, such as a C-arm X-ray machine, can be used to capture two-dimensional images of bony anatomy of the patient 104. An endoscopic imaging system 111 can be used to image a surgical space within the patient 104. Endoscopic imaging system 111 may include an endoscopic imager 112 (which may include an endoscope removably attached to an endoscopic camera head) and one or more additional endoscopic imaging system components 114, such as a light source for providing light to a surgical cavity via the endoscope of the endoscopic imager 112 and a camera control unit (CCU) that is configured to receive imaging data from the camera head and generate endoscopic images.

Endoscopic imager 112 includes one or more sensors and/or light providers. FIG. 1B illustrates a functional block diagram illustrating exemplary sensing and light providing functional features of an example of endoscopic imager 112. Endoscopic imager 112 includes one or more image sensors 140. Image sensor(s) 140 may be used for imaging a surgical space within a patient. Endoscopic imager 112 may optionally include one or more additional sensors 142. For example, sensors 142 may include one or more inertial measurement units (IMUs). The IMU(s) may be attached to endoscopic imager 112 and may be configured to track movement of endoscopic imager 112. Alternatively, or additionally, sensors 142 may include one or more time-of-flight sensors configured to detect light emitted by a time-of-flight light provider 146.

Endoscopic imager 112 also includes one or more light provider components. For example, endoscopic imager 112 includes an imaging light provider 144 for providing the light necessary for illuminating a surgical space for imaging. Imaging light provider 144 may be the optical components of the endoscopic imager 112 that provide a light pathway for externally provided light, such as light from endoscopic light source(s) 112. Alternatively, imaging light provider 144 may include one or more light sources configured to generate light for illuminating the surgical space. Optionally, endoscopic imager 112 may include a time-of-flight light provider 146. Time-of-flight light provider 146 may be configured to direct concentrated light beams at bony anatomy in order to generate a three-dimensional model of bony anatomy using time-of-flight techniques. In some examples, time-of-flight light provider 146 may be a laser.

Returning to FIG. 1A, the system 100 may include one or more displays 120 on which the surgeon can view information related to a surgical procedure. For example, two-dimensional images of bony anatomy captured by the two-dimensional imaging system 110 and/or endoscopic images (e.g., single snapshot images and/or video) captured using endoscopic imaging system 111 can be displayed on the one or more displays 120. A three-dimensional model of the bony anatomy may also be displayed on the one or more displays 120. One or more visual indicators may be displayed on one or more displays 120 to guide a user (e.g., a surgeon) in removing bone during the surgical procedure. For example, visual indicators that provide real-time guidance on bone removal may be displayed to the surgeon. The visual indicators may include, for example, arrows (or any other suitable graphical indicators) overlaid on the three-dimensional model that point the surgeon toward a bony protrusion. The visual indicators may be dynamic (e.g., arrows pointing to a bony protrusion may change color depending on the proximity of a surgical tool to the bony protrusion). Alternatively, or additionally, a checklist for a surgical procedure can be displayed on the one or more displays 120. For example, a checklist can include a list of standard leg positions in which a surgeon should image a patient's leg during a given surgical procedure and/or a series of prompts for the surgeon regarding bone to be resected. A display 120 can be any suitable type of display, including but not limited to a boom-mounted display, a display on an imaging cart, a touchscreen display of a tablet or smartphone, and an augmented or virtual reality display.

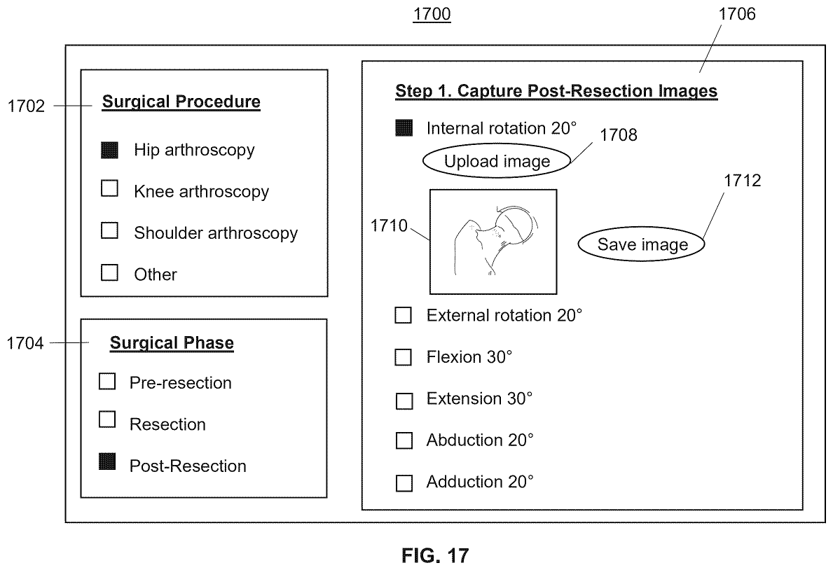

As noted above, the one or more displays 120 can display a checklist for a surgical procedure. The checklist can include a list of steps in the procedure. According to an aspect, the one or more displays 120 can display a graphical user interface including the checklist, such that a user can interact with the checklist to view, add, and edit information associated with the various steps of the procedure. FIG. 17 illustrates an exemplary graphical user interface 1700 displaying a checklist for guiding a surgeon through a surgical procedure. The graphical user interface 1700 includes a procedure type panel 1702 that includes a list of surgical procedures (e.g., hip arthroscopy, knee arthroscopy, shoulder arthroscopy, other). A user may select an indicator (e.g., a checkbox) next to the type of surgical procedure that is ongoing. In the example of FIG. 17, the indicator for a hip arthroscopy has been selected. In accordance with the selected surgical procedure type, a procedure stage panel 1704 may populate with a list of stages in the selected surgical procedure. The user may select an indicator next to the phase of the surgical procedure that is ongoing. In the example of FIG. 17, the indicator for the post-resection stage has been selected.

Graphical user interface 1700 further includes an instruction panel 1706 that displays instructions for the selected surgical phase. For example, in FIG. 17, instruction panel 1706 displays an instruction to capture post-resection images based on the selection of the “post-resection” surgical phase in procedure stage panel 1704. The instruction includes a list of positions in which the anatomy of interest should be imaged during the post-resection phase. When a position is selected, the user may be presented with the option 1708 to upload an image associated with that position. The uploaded image 1710 may be displayed to the user. If the user is satisfied with the image 1710, the user may be presented with the option 1712 to save the image before proceeding to the next position. While FIG. 17 illustrates an exemplary checklist including instructions for a post-resection stage, it should be understood that similar checklists may be used for the pre-resection stage and post-resection stages, according to various embodiments.

System 100 may include an image processing system 106 that is configured to generate visualizations for display on the one or more displays 120 for guiding a surgical procedure on the patient 104. As used herein, an “image processing system” refers to any computing system configured to process image data as well as non-image data, such as data from one or more sensors. For example, image processing system 106 may receive data from one or more inertial measurement units (IMUs) 118 attached to a surgical tool, endoscopic imager, or bony anatomy. The image processing system 106 may include one or more processors. The image processing system 106 may be communicatively connected to the endoscopic imaging system 111 and to the two-dimensional imaging system 110 to receive and process images from the endoscopic imaging system 111 and two-dimensional imaging system 110. Image processing system 106 may generate visualizations for guiding bone removal that display at least a portion of a three-dimensional model of bony anatomy that is associated with planned bone removal for the bony anatomy. The three-dimensional model may be stored in a memory of the image processing system 106, received directly from another imaging system, or received via a USB flash drive or other transportable storage device inserted into, or otherwise coupled to, image processing system 106. According to some aspects, the three-dimensional model may be transmitted to image processing system 106 wirelessly, such as through WiFi or a different type of wireless network. Alternatively, the image processing system 106 may obtain the three-dimensional model from a remote system 108. Remote system 108 may be a cloud storage system, a data repository within the network of the treatment facility in which system 100 is located, a data repository outside of the network of the treatment facility, or any other suitable system for storing three-dimensional model data. The visualizations may include the three-dimensional model displayed in a position and orientation that aligns with the bony anatomy as captured in an endoscopic image generated by endoscopic imaging system 111.

The image processing system 106 may be configured to determine an alignment of a three-dimensional model relative to an endoscopic image captured by endoscopic imaging system 111 in order to generate visualizations for guiding bone removal in which the three-dimensional model is displayed based on its alignment relative to the endoscopic image. As discussed in detail below, the determination of the alignment of the three-dimensional model relative to the endoscopic image may be based on one or more two-dimensional images captured by two-dimensional imaging system 110. Image processing system 106 may generate one or more visualizations based on the determined alignment of the three-dimensional model relative to the endoscopic image, such as an overlay image in which the three-dimensional model is overlaid on the endoscopic image, a side-by-side view of the three-dimensional model and the endoscopic image, and/or a simulated endoscopic image based on the three-dimensional model. Image processing system 106 may provide the visualizations to one or more displays 120.

The visualizations may represent the bony anatomy as it exists at a given moment in time. Alternatively, as noted above, the visualizations can be used to guide a surgeon during a bone removal procedure and may represent a real-time view of the bony anatomy. Image processing system 106 may update the visualizations to reflect changes to the bony anatomy as a bone removal instrument 116 removes bone. The movement of bone removal instrument 116 may be tracked using endoscopic imaging system 111 and/or an IMU 118 associated with the bone removal instrument (e.g., affixed to the bone removal instrument). Image processing system 106 may receive the tracking information and update the visualizations accordingly. Optionally, image processing system 106 may also update the visualizations based on tracking the movement of endoscopic imager 112 and/or the bony anatomy of patient 104. For example, one or more IMUs 118 may be attached to endoscopic imager 112 and/or the bony anatomy of patient 104 to monitor their respective movements, and data from the IMUs 118 may be provided to image processing system 106 to update the visualizations for display 120.

A user control device 122 may be communicatively coupled to image processing system 106. User control device 122 may have user input/output functionality, such as a touchscreen, touchpad, trackball, keyboard, mouse, gesture recognition device, voice activation feature, or pupil reading device. A surgeon may use user control device 122 to interact with what is displayed on the display 120. For instance, the surgeon may select one or more visualizations for use in guiding the surgeon during the surgical procedure. User control device 122 may also be configured to allow the surgeon to customize aspects of the visualizations, for example by adjusting the transparency of an overlay image, zooming in or out on a visualization, or displaying a visualization of planned bone removal on an image. User control device 122 may be at least partially located in the sterile field, such that the surgeon may control aspects of display 120 during the surgical procedure. For example, user control device 122 may comprise a touchscreen tablet mounted to surgical table 102 or to a boom-type tablet support and covered with a sterile drape to maintain the surgeon's sterility as he or she operates the touchscreen tablet.

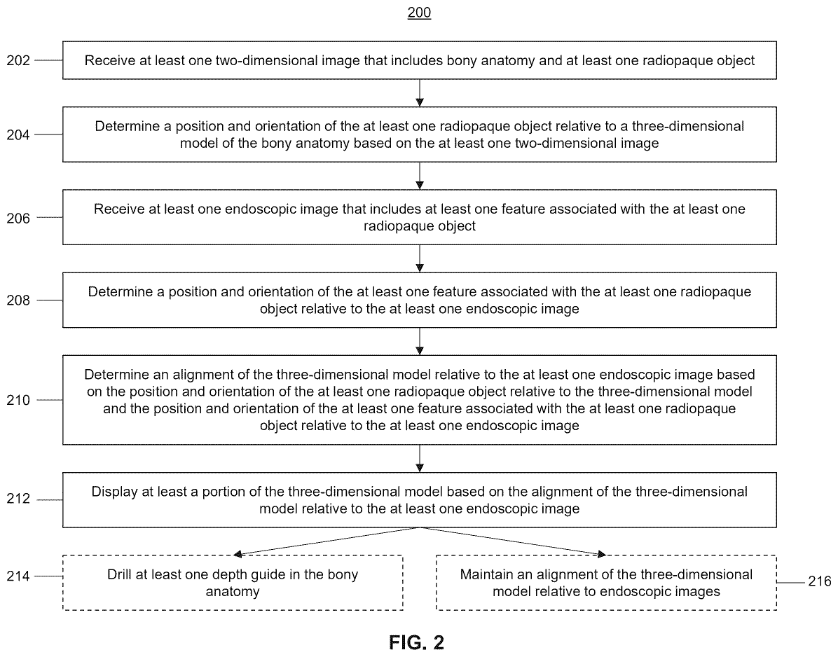

As noted above, image processing system 106 may be configured to generate visualizations that can be displayed via one or more displays 120 to guide a surgeon during a bone removal procedure. FIG. 2 illustrates an exemplary method 200 that may be performed by one or more processors of image processing system 106, or any other suitable computing system, for guiding a surgeon in the removal of bone from a portion of a joint during a surgical procedure. For example, performing method 200, image processing system 106 may generate a visualization for display by display 120 that includes at least a portion of a three-dimensional model of bony anatomy displayed in a position and orientation that aligns with the bony anatomy in an endoscopic image. Method 200 includes determining the alignment of the three-dimensional model relative to the endoscopic image and displaying at least a portion of the three-dimensional model based on the determined alignment so that a surgeon can better associate the bony anatomy as a whole with the limited view that the surgeon sees in the endoscopic image. In some aspects, the displayed three-dimensional model may include a visualization of planned bone removal to indicate to the surgeon where and how much bone should be removed from various portions of the bony anatomy. This can be advantageous to the surgeon by enabling the surgeon to better associate the planned bone removal with the endoscopic image. Method 200 may be used for any joint of the body, including the hip joint, shoulder joint, knee joint, spinal joints, etc., and/or for any anatomy of the body.

At step 202, at least one two-dimensional image comprising bony anatomy and at least one radiopaque object is received. The at least one two-dimensional image may be a fluoroscopic image received, for example, by image processing system 106 from two-dimensional imaging system 110 of FIG. 1A. The two-dimensional image(s) may include the portion of the bone that is being or will be surgically treated, as well as surrounding portions of the bone that enable the surgeon to compare what is shown in the two-dimensional image(s) to what the surgeon sees endoscopically. For instance, in examples involving debridement of a bony pathology (e.g., a cam pathology), the two-dimensional image(s) may include the head and neck of the femur. The two-dimensional image(s) may be pre-generated (i.e., generated prior to performance of method 200) and retrieved from a suitable image storage system. Pre-generated two-dimensional images may be captured when the patient is in the same position as they are when method 200 is performed (e.g., immediately prior to the commencement of method 200) so that the pre-generated two-dimensional images accurately reflect the position and orientation of the patient's bony anatomy at step 202. According to various aspects, the two-dimensional images may be captured earlier and/or when the patient is not in exactly the same position and orientation as they are when method 200 is performed.

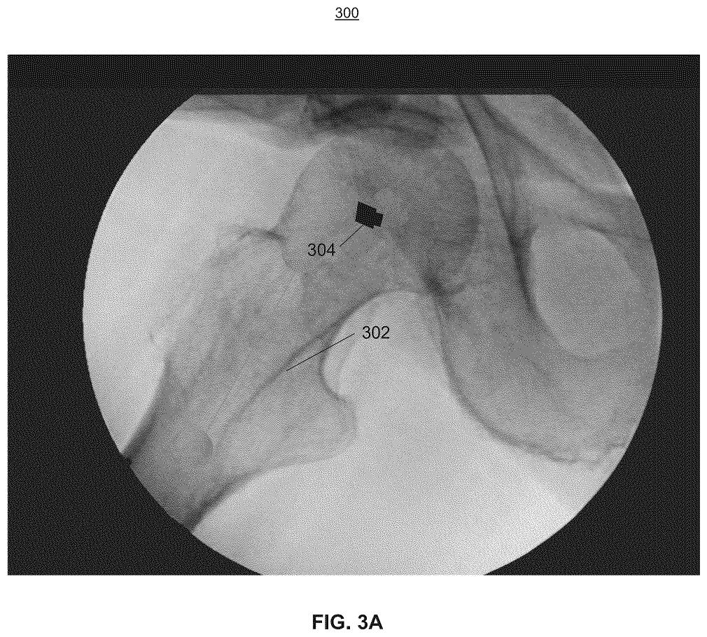

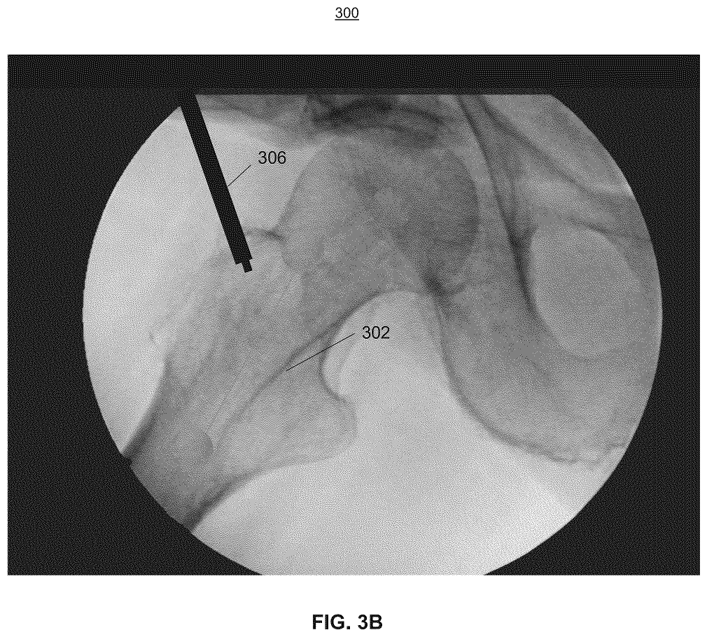

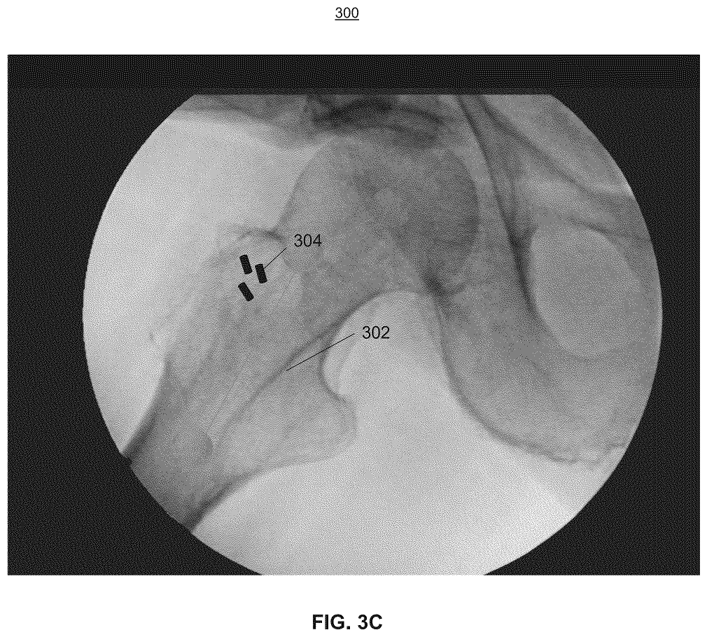

The two-dimensional image(s) also include at least one radiopaque object. The at least one radiopaque object may be an object that is affixed to or otherwise positioned on the bony anatomy. For example, the object may be inserted into (e.g., screwed into) the bony anatomy prior to step 202. The at least one radiopaque object may be an object with a known geometry. FIG. 3A illustrates an exemplary two-dimensional image 300 that includes the bony anatomy 302 and an exemplary radiopaque object 304 that has a known geometry. Radiopaque object 304 may be, for example, an irregularly shaped polygon that is affixed to bony anatomy 302. As discussed in greater detail below, image processing system 106 may use the known geometry of the radiopaque object 304 to determine the position and orientation of radiopaque object 304 relative to the two-dimensional image 300, and subsequently to a three-dimensional model of the bony anatomy 302. For example, a three-dimensional model of radiopaque object 304 may be stored in a memory of image processing system 106 or in remote system 108. Image processing system 106 may retrieve the three-dimensional model of radiopaque object 304 and use the three-dimensional model of radiopaque object 304 to determine a position and orientation of radiopaque object 304 relative to a three-dimensional model of bony anatomy 302.

As noted above, the radiopaque object(s) may include an object that is affixed to the bony anatomy. Alternatively, the at least one radiopaque object may include an object that is not affixed to bone. For example, the at least one radiopaque object may include a radiopaque tool. FIG. 3B illustrates an exemplary endoscopic image 300 that includes bony anatomy 302 and a radiopaque tool 306. Radiopaque tool 306 may be a tool for positioning objects on or in bony anatomy 302, a pointer tool, a tool for removing tissue (e.g., a bur, drill, or other bone removal instrument), or any other tool used during the surgical procedure.

The radiopaque object(s) may be radiopaque object(s) that do not have a known geometry. As used herein, “known geometry” refers to geometry that is known by image processing system 106 and used in method 200. As such, while the geometry of a small pin may be known in the abstract, its geometry may not be “known” to the image processing system 106. FIG. 3C illustrates an example in which the at least one radiopaque object is a plurality of small radiopaque objects 304, such as pins or beads, whose geometry is not known to image processing system 106. The relative arrangement of the radiopaque objects 304 may also not be known in the sense that the radiopaque objects 304 may have been placed in relatively arbitrary locations of the bony anatomy such that their positions relative to one another are not initially known to image processing system 106. For example, the radiopaque objects 304 may be placed by a surgeon using an insertion tool after a three-dimensional model of the bony anatomy has already been generated.

Optionally, one or more pre-processing operations may be applied to the two-dimensional image(s), such as one or more scaling operations, cropping operations, down-sampling, up-sampling, etc. A dewarping operation may be applied to correct for warping caused by two-dimensional imaging system 110. Dewarping of a given two-dimensional image may be performed based on the determined relationship between a known pattern of reference markers attached to the detector of two-dimensional imaging system 110 and the reference markers visible in the two-dimensional image. For example, the reference markers in a two-dimensional image may be detected, a non-rigid transformation that maps the known positions of the reference markers to the markers visible in the image may be calculated, and the transformation may be applied to the image, resulting in a dewarped image. The reference markers may then be removed from the image.

Optionally, multiple two-dimensional images may be received, and the two-dimensional images may depict the bony anatomy in a plurality of positions. Depicting the bony anatomy in a plurality of positions may be useful in providing multiple views of the bony anatomy from multiple different angles and/or showing relative positions of the bony anatomy relative to other anatomical features. To increase the amount of the bony anatomy that is viewable with the camera, the leg can be moved into a plurality of different positions. The plurality of positions may include predetermined positions, such as standard positions that are used during a surgical procedure. The standard positions of the leg include particular degrees or ranges of degrees of internal rotation, external rotation, flexion, extension, abduction, and/or adduction. During a hip surgery, the two-dimensional images received at step 202 may include images of the bony anatomy in at least two of the standard positions in order to provide the surgeon with different views of the hip joint. Alternatively, the received two-dimensional images may include images of the bony anatomy in a combination of standard and non-standard positions. For example, the received two-dimensional images may include images of the bony anatomy in at least one of the standard positions and at least one non-standard position. Alternatively, the received two-dimensional images may include images of the bony anatomy in a plurality of non-standard positions. Alternatively, the received two-dimensional images may include images of the bony anatomy in the same position.

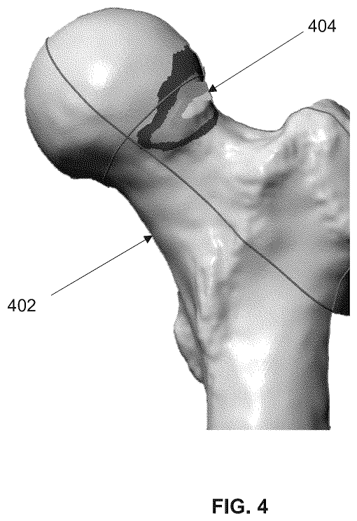

At step 204, a position and orientation of the at least one radiopaque object relative to a three-dimensional model of the bony anatomy is determined based on the at least one two-dimensional image. The three-dimensional model of the bony anatomy may represent at least a portion of the joint that is targeted for surgical treatment. FIG. 4 illustrates an example of a three-dimensional model 402 of a portion of a femur. The three-dimensional model 402 may be generated based on a pre-operative three-dimensional scan of the patient's joint. For example, for a hip surgery, a patient's hip joint can be imaged, and a model can be built from the imaging that includes various portions of the hip joint, including, for example, the femoral head, the femoral neck, the acetabular cup, the pelvis, femoral condyles, etc. The joint may be imaged using any suitable imaging system for generating three-dimensional image data, including, for example, a CT imaging system, an ultrasound imaging system, a C-arm imaging system, a cone beam CT imaging system, or an MRI imaging system. The three-dimensional model 402 may be pre-generated and stored for use during the surgical session (e.g., by remote system 108 of FIG. 1A). Alternatively, the three-dimensional model 402 may be generated intraoperatively, such as by an intraoperative CT system or by combining information from multiple two-dimensional images (e.g., multiple images captured by a C-arm imaging system) to construct the three-dimensional model 402. The three-dimensional model 402 may be updated based on images captured during the surgical procedure to reflect the removal of bone during the surgical procedure.

According to an aspect, the three-dimensional model 402 may be generated by mapping the bony anatomy using a tracked surgical instrument. A surgical instrument (e.g., a pointer tool, a bone removal tool such as a bur or shaver tool, or any other surgical tool) may be tracked as the surgical instrument is touched to a plurality of locations on the bony anatomy. In some implementations, the tip of the tracked surgical instrument may be gently dragged across the surface of the bony anatomy of interest in order to define the contours of the surface. Alternatively, the tip of the tracked surgical instrument may not be dragged cross the entire surface but instead may be touched to a plurality of points on the surface of the bony anatomy. In other implementations, the tracked surgical instrument may be a surgical instrument that is configured to grasp the bony anatomy rather than a surgical instrument that is dragged across or touched against the bony anatomy. For instance, the tracked surgical instrument may be a pointer tool with a claw-shaped distal end portion that can wrap around at least a portion of the bony anatomy, and touching the tracked surgical instrument to the bony anatomy may include wrapping the distal end of the tracked surgical instrument around a portion of the bony anatomy. Once the tracked surgical instrument has been touched to the bony anatomy, one or more surfaces of the bony anatomy may be estimated to generate the three-dimensional model 402 based on the locations touched by the tracked surgical instrument.

In some examples, computer vision techniques may be used to track the surgical instrument in order to generate the three-dimensional model 402. For example, the surgical instrument may be tracked by applying a visual simultaneous localization and mapping (vSLAM) algorithm to endoscopic video of the surgical instrument. The vSLAM algorithm may be configured to detect the surgical instrument within received endoscopic video and track the surgical instrument across frames. Based on analysis of the tracked movement of the surgical instrument across frames, the vSLAM algorithm can generate the three-dimensional model 402. In some aspects, one or more other computer vision techniques may be used to analyze the endoscopic video of the surgical instrument in order to track the position of the surgical instrument, such as one or more object tracking algorithms, optical flow techniques, monocular depth estimation techniques, or any other suitable techniques.

Alternatively, or additionally, the tracked surgical instrument may be tracked by a camera external to the subject. The camera may be an RGB color camera, an infrared camera, or any other type of camera. The camera may track a fiducial marker associated with the surgical instrument that is external to the subject. For example, the surgical instrument may include a visual fiducial. The visual fiducial may be located on a portion of the surgical instrument that is external to the subject and thus outside of the field of view of an endoscopic imager. The visual fiducial may be an ArUco, QR code, or any other visual pattern or marking. The visual fiducial may be tracked by a camera external to the subject to determine the movement of the surgical instrument, which can be used to determine a surface of bony anatomy touched by the surgical instrument.

Optionally, one or more vSLAM algorithms may be used to analyze video captured by the camera external to the subject in order to generate the three-dimensional model 402. For example, one or more vSLAM algorithms may be used to track the movement of the visual fiducial across a sequence of video frames. This information can be used to determine a surface of bony anatomy touched by the surgical instrument to which the visual fiducial is attached, which can be used as or can be used to determine the three-dimensional model 402. In some examples, an alert may be provided to a user (e.g., a surgeon or other medical personnel) if the subject has moved while the video is being captured. The alert may prompt the user to re-register the three-dimensional model 402 with respect to the bony anatomy, such as by touching the surgical instrument that includes the visual fiducial to one or more locations on the bony anatomy whose position is known relative to the three-dimensional model 402. In some aspects, one or more other computer vision techniques may be used to analyze video captured by a camera external to the subject, such as one or more object tracking algorithms, optical flow techniques, monocular depth estimation techniques, or any other suitable techniques.

In some examples, computer vision techniques may be used to track features associated with the bony anatomy rather than a surgical instrument in order to generate the three-dimensional model 402. For example, any one or more of the computer vision techniques discussed above (e.g., vSLAM) may be used to track at least one fiducial, bone marking, bur pocket, piece of wax or gel, bead, etc. that is associated with (e.g., attached to or formed on or in) the bony anatomy and determine its motion across frames in order to generate the three-dimensional model 402. Alternatively or additionally, computer vision techniques may be used to track the position of the bony anatomy itself, which may help a surgeon to understand the position of the bony anatomy. According to an aspect, the computer vision techniques may be used in combination with one or more other techniques.

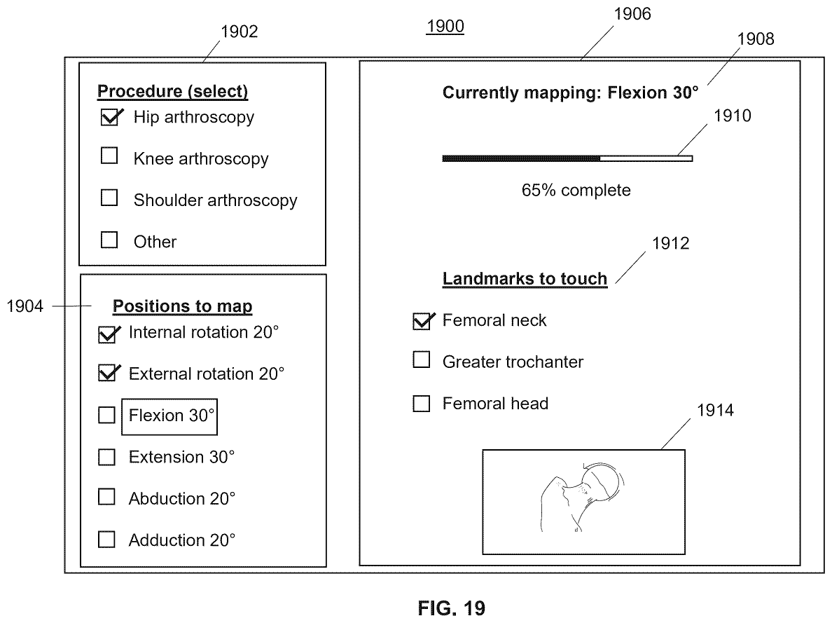

A surgeon may receive guidance regarding which and how many locations on the bony anatomy to touch with the tracked surgical instrument. For example, a graphical user interface may be displayed (e.g., via display(s) 120 of system 100) that guides the surgeon to touch the tracked surgical instrument to particular areas based on the type of bony anatomy being modeled. FIG. 19 illustrates an exemplary graphical user interface 1900 for guiding a surgeon in touching a tracked surgical instrument to bony anatomy in order to generate a three-dimensional model. Graphical user interface 1900 includes a procedure type panel 1902. Procedure type panel 1902 may include a list of surgical procedures (e.g., hip arthroscopy, knee arthroscopy, shoulder arthroscopy, other), and a surgeon may indicate which type of procedure is ongoing, for example by operating user control device 122 to check a checkbox corresponding to the appropriate surgical procedure (e.g., via a button press). A position panel 1904 may populate with a list of positions in which the bony anatomy should be placed for touching the bony anatomy with the tracked surgical instrument based on the procedure type selected in procedure panel 1902. The surgeon may check boxes corresponding to positions in which the bony anatomy has already been touched with the tracked surgical instrument. The surgeon may also select the current position of the bony anatomy. For example, in FIG. 19, position panel 1904 indicates that the leg is in a 30° flexion position.

Graphical user interface 1900 further includes a status panel 1906 that includes information about how much of the bony anatomy in the current position has been touched with the tracked surgical instrument and how much more needs to be touched in order to collect enough information to generate a three-dimensional model. The amount of information needed to generate a three-dimensional model may be predetermined. For example, the system may be configured to generate a three-dimensional model of the bony anatomy using at least 10, 20, 30, 40, 50, 60, 70, 80, 90, 100, or more unique points on the bony anatomy. As shown in FIG. 19, status panel 1906 includes a position indicator 1908 showing a current position of the bony anatomy and a corresponding progress indicator 1910. Progress indicator 1910 may indicate the current amount of information about the bony anatomy gathered based on touching the tracked surgical instrument to the bony anatomy relative to the amount of information required to generate a three-dimensional model.

Optionally, status panel 1906 may include a list 1912 of landmarks to touch with the tracked surgical instrument. A surgeon may indicate when a landmark has been touched with the tracked surgical instrument by checking a corresponding checkbox. For example, in the example shown in FIG. 19, the femoral neck has been touched with the tracked surgical instrument, but the greater trochanter has yet to be touched. Status panel 1906 may also include a preliminary version of a three-dimensional model 1914 generated based on touching the tracked surgical instrument to the bony anatomy. The preliminary version of the three-dimensional model 1914 may update in real-time as the tracked surgical instrument is touched to the bony anatomy. The preliminary version of the three-dimensional model 1914 may also provide real-time feedback to the surgeon regarding additional landmarks that should be touched in order to build a detailed three-dimensional model. For example, the display of the preliminary version of the three-dimensional model 1914 may include one or more text alerts and/or visual indications on the preliminary version of the three-dimensional model 1914 (e.g., outlining, highlighting, etc.) that identify areas and/or landmarks of the bony anatomy that should be touched in order to improve the three-dimensional model.

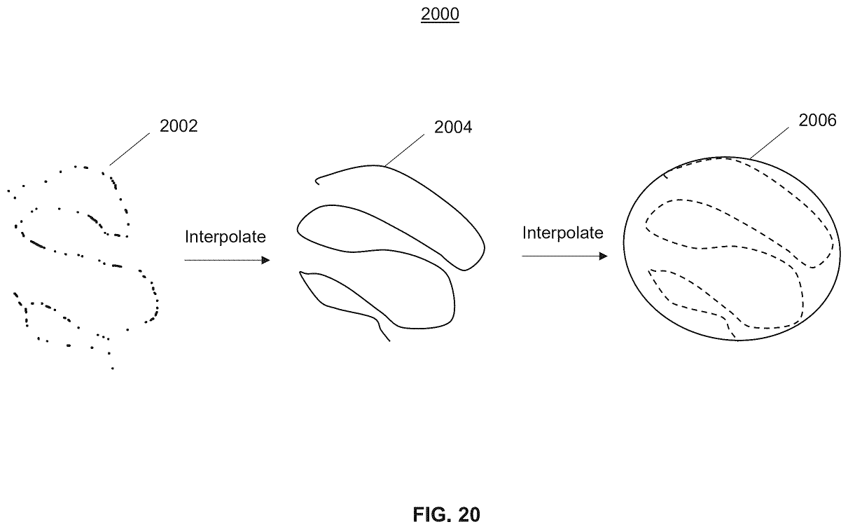

One or more surfaces can be estimated by stitching together the three-dimensional coordinates generated by touching the tracked surgical instrument to a plurality of locations on the bony anatomy in order to generate the three-dimensional model 402. FIG. 20 illustrates an example of how a surface can be determined by stitching together a plurality of coordinates corresponding to locations of the bony anatomy touched by the tracked surgical instrument. A plurality of coordinates 2002 is collected by touching a tracked surgical instrument to bony anatomy. The plurality of coordinates 2002 may form the rough outline of a surface of bony anatomy, but because the tracked surgical instrument is not touched to every point on the surface of the bony anatomy, there may be gaps between coordinates. A surface 2004 may be generated by interpolating the gaps between coordinates. For example, areas corresponding to untouched areas of the bony anatomy may be interpolated using a machine learning model trained to estimate the contours of bony anatomy based on a limited number of coordinates. In some examples, the interpolation performed by the machine learning model may be based at least partially on the position of the bony anatomy in which the coordinates were collected. Alternatively, or additionally, the interpolation may be based at least partially on motion of the camera used to track the surgical instrument as it was touched to the bony anatomy (e.g., an endoscopic camera or a camera external to the patient). For example, the motion of the camera may be determined using one or more inertial measurement units attached to the camera and/or by using optical flow techniques to analyze pixel motion across sequential image frames captured by the camera, and the motion of the camera may be used to inform the interpolation performed by the machine learning model. According to another aspect, the interpolation may be based on a statistical shape model of the bony anatomy. For example, the plurality of coordinates may be compared to a statistical shape model, and the statistical shape model may be used to fill in gaps between existing coordinates.

A surface determined by stitching together a plurality of coordinates may be the three-dimensional model 402. Alternatively, the three-dimensional model 402 can be generated based on the determined surface. For example, in FIG. 20, surface 2004 may be transformed into a three-dimensional model of a femoral head 2006 by interpolating additional aspects of the bony anatomy based on surface 2004. In some examples, three-dimensional model 2006 may be generated by providing surface 2004 to a trained machine learning model configured to generate three-dimensional models of bony anatomy based on partial representations of the bony anatomy. Alternatively, or additionally, a three-dimensional model may be generated by stitching together multiple determined surfaces. For example, a given determined surface may represent only a small portion of the bony anatomy, and multiple surfaces may need to be determined to generate a fuller three-dimensional model of the bony anatomy.

According to another aspect, the three-dimensional model 402 may be generated based on two-dimensional image data (e.g., X-ray image frames) captured from different perspectives. For example, first two-dimensional image data that captures a joint from a first perspective and second two-dimensional image data that captures the joint from a second perspective may be received. The first and second image data may be generated via a two-dimensional imaging modality, such as a C-arm imaging system. Three-dimensional image data may be generated by back-projecting the first and second two-dimensional image data in three-dimensional space in accordance with a relative difference between the first and second imaging perspectives. The three-dimensional model 402 may then be generated based on the three-dimensional image data. For instance, the three-dimensional image data may be processed using a machine learning model to generate a set of multi-class voxels. Each multi-class voxel may represent bone or no-bone. The machine learning model may be trained on imaging data generated via at least a three-dimensional imaging modality (e.g., CT, MRI). The three-dimensional model 402 may then be generated based on multi-class voxels of the set of multi-class voxels that correspond to a class associated with the joint (e.g., voxels that represent bone).

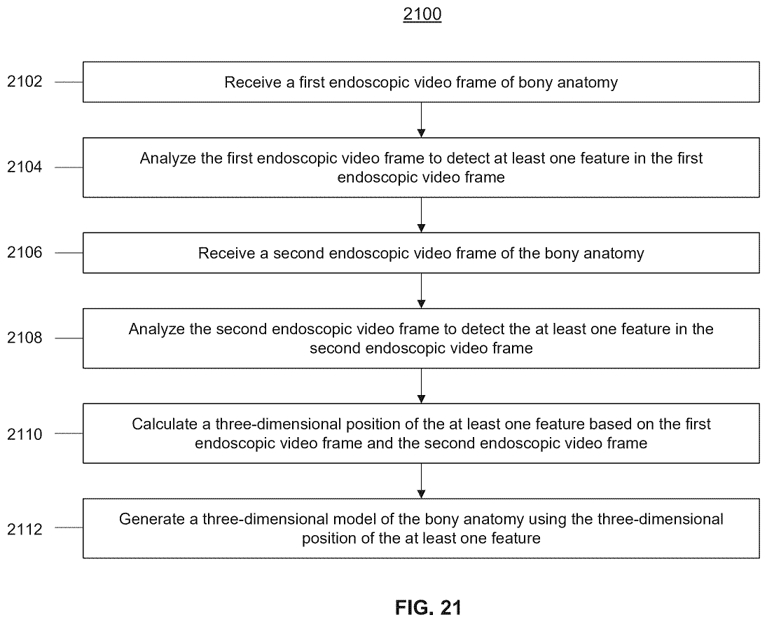

According to another aspect, the three-dimensional model 402 may be generated by analyzing a series of endoscopic video frames depicting the bony anatomy to identify features of the bony anatomy and track their motion across video frames. The tracked motion of the features can be used to triangulate three-dimensional points, which can be used to build the three-dimensional model 402. An exemplary method 2100 for generating a three-dimensional model (e.g., three-dimensional model 402) is illustrated in FIG. 21. Method 2100 can be performed by one or more processors, such as one or more processors of image processing system 106.

At step 2102, a first endoscopic video frame is received. The endoscopic video frame includes the bony anatomy for which a three-dimensional model is desired. At step 2104, the first endoscopic video frame is analyzed to detect at least one feature in the first endoscopic video frame. The at least one feature may be any aspect of the first endoscopic video frame that can be tracked across successive video frames. For example, the first endoscopic video frame may be analyzed to detect one or more edges, corners, texture patterns, or shapes in the frame.

At step 2106, a second endoscopic video frame is received. The second endoscopic video frame includes the same bony anatomy pictured in the first endoscopic video frame but from a slightly different perspective. For example, the second endoscopic video frame may be a successive video frame in a series of video frames captured while moving the endoscopic camera around within a surgical space. At step 2108, the second endoscopic video frame is analyzed to detect the at least one feature in the second endoscopic video frame. The position and orientation of the at least one feature within the second endoscopic video frame may be different relative to its position and orientation within the first endoscopic video frame due to the movement of the camera between frames.

At step 2110, a three-dimensional position of the at least one feature is calculated based on the first endoscopic video frame and the second endoscopic video frame. The three-dimensional position of the at least one feature may be calculated based on the difference in the location of the at least one feature between frames. For example, optical flow techniques may be used to estimate the motion of the endoscopic camera between frames based on the apparent motion of pixels between the first and second endoscopic video frames. The position and orientation of the endoscopic camera when the first endoscopic video frame was captured may serve as a reference coordinate. A position and orientation of the endoscopic camera when the second endoscopic video frame was captured may be calculated relative to the reference coordinate based on the estimated motion of the endoscopic camera. Based on the estimated three-dimensional positions of the endoscopic camera when each video frame was captured and the position and orientation of the at least one feature in each video frame, the three-dimensional position of the at least one feature may be triangulated.

At step 2112, a three-dimensional model of the bony anatomy is generated using the three-dimensional position of the at least one feature. The three-dimensional position of the at least one feature may be used to initialize a map of the bony anatomy. Steps 2102-2112 may then be repeated with successive video frames in order to calculate the three-dimensional positions of various features. Optionally, steps 2102-2112 may be repeated by calculating the positions of multiple features. Steps 2102-2112 may be repeated for any number of video frames (e.g., two, three, four, five, or more). The three-dimensional position of each feature may be successively added to the map. The map may be the three-dimensional model (e.g., three-dimensional model 402).

Optionally, information about the tissue surrounding the bony anatomy may be used to generate the three-dimensional model 402. The tissue information may be used to supplement the information about the bony anatomy gathered using any of the techniques described herein. Generating the three-dimensional model 402 may include identifying at least one type of tissue associated with the bony anatomy, such as a ligament, tendon, cartilage, fibrocartilage, muscle, and/or bone.

The at least one type of tissue may be identified using a database of tissue types. A representation of the bony anatomy (e.g., a preliminary version of a three-dimensional model) may be compared to a database of tissue types in order to determine one or more tissue types present in the representation of the bony anatomy. Identifying types of tissue may help to identify the bony anatomy being mapped. For example, the arrangement of ligaments, tendons, cartilage, fibrocartilage, muscle, and/or bone in the representation of the bony anatomy may indicate that the bony anatomy is a hip. The indication that the bony anatomy is a hip may be used to generate the three-dimensional model 402. For example, the generated three-dimensional model 402 may be compared to a generic three-dimensional model of a hip to ensure that the generated three-dimensional model roughly matches the expected form of a hip.

Alternatively or additionally, the at least one type of tissue may be identified using machine learning techniques. For example, a machine learning model may be configured to receive endoscopic images of anatomy and identify the types of tissue present in the endoscopic image. Optionally, the machine learning model may be configured to receive multi-spectral imaging data that includes images captured within various wavelength ranges across the electromagnetic spectrum. The machine learning model may be configured to analyze the multi-spectral imaging data to identify tissue shown in the images. Multi-spectral imaging data may provide a more comprehensive view of the anatomy due to the images in different spectral bands capturing different details about the anatomy, which may improve the accuracy of the tissue identification process.

Alternatively or additionally, the at least one type of tissue may be identified by comparing a representation of the bony anatomy to a pre-operatively generated model of the bony anatomy that includes tissue information. The pre-operatively generated model may be a subject-specific model that is generated from pre-operative images such as MRI or ultrasound images, which may provide a detailed view of the various tissues associated with the imaged bony anatomy. Alternatively, the pre-operatively generated model of the bony anatomy that includes tissue information may not be subject-specific. For example, the pre-operatively generated model of the bony anatomy may be a statistical shape model of the bony anatomy that includes tissue information. Using tissue information to generate the three-dimensional model 402 may improve the accuracy of the three-dimensional model 402. For example, tissue information may be used to confirm the accuracy of the locations of various anatomical structures in the three-dimensional model 402.

The three-dimensional model 402 may include a representation of at least one region of the portion of the joint that deviates from a target morphology. The three-dimensional model can be used to assist a practitioner in planning for a surgical procedure on region(s) of the joint, such as by including indications of where and how much bone should be removed. For example, a three-dimensional model of a portion of a hip joint of a subject can be generated that includes information identifying a location of a hip joint pathology (e.g., a condition or a disorder), and an amount of bone that may be removed to match a baseline or “target” anatomy. In the illustrated example, the three-dimensional model 402 includes a heat map 404 covering a hip joint pathology. Heat map 404 indicates the location and amount of bone that may be removed from the hip joint pathology to match a target morphology. Heat map 404 includes variation in color that indicates a degree of deviation from the target morphology. In other examples, heat map 404 may include variations in contrast, shading, pattern, or any other suitable visual indicator to indicate a degree of deviation from the target morphology. A user, such as the surgeon or a third party, can tailor the settings of heat map 404 for surgical planning purposes, such as by altering one or more parameters that determine the deviations from the target bone morphology, which can increase or decrease the size of the region indicated for bone removal and/or increase or decrease the amount of bone indicated for removal. It should be understood that heat map 404 is merely illustrative and other types of indications of planned bone removal may be displayed. For example, an indication of planned bone removal may include a map (e.g., a contour map) that defines a plurality of regions and that indicates the depth of planned bone removal in those regions. The depth of each region may be indicated by using different types of outlining to identify the boundaries of the region and/or by labeling each region with a numerical depth value.

Various aspects of heat map 404 may be user-adjustable. For example, a user may utilize user control device 122 to edit a heat map 404 displayed by display 120. The user may adjust the positions of outlines indicating different regions of the heat map 404, adjust the color scheme of the heat map 404, and/or reassign a new depth of planned bone removal to a region of the heat map 404. Other types of indications of planned bone removal may be similarly adjustable. For example, in implementations where the indication of planned bone removal is a contour map, the user may adjust the boundaries displayed on the contour map and/or reassign a new depth of planned bone removal to a given region.

In some examples, three-dimensional model 402 may include only the bony anatomy of a joint, as shown in FIG. 4. Alternatively, three-dimensional model 402 may include the cartilage layer of the joint. Information about the cartilage layer may be obtained using MRI imaging or any other imaging modality that is configured to visualize cartilage. In some implementations, if information about the cartilage layer of the joint is available, display of the cartilage layer in three-dimensional model 402 may be toggled on and off by a user. Optionally, the indication of planned bone removal (e.g., heat map 404) may include the cartilage layer, and the indication of planned bone removal may indicate the location and depth of cartilage to be removed. The display of the cartilage layer in the indication of planned bone removal may also be toggled on and off by a user. According to various embodiments, the indication of planned bone removal (e.g., heat map 404) may include the cartilage layer, while the three-dimensional model 402 does not include the cartilage layer. Similarly, the three-dimensional model 402 may include the cartilage layer, while the indication of planned bone removal (e.g., heat map 404) does not include the cartilage layer.

The position and orientation of the at least one radiopaque object relative to the three-dimensional model may be determined using the at least one two-dimensional image received at step 202. The position and orientation of the at least one radiopaque object relative to the three-dimensional model may be determined based on a position and orientation of the at least one radiopaque object relative to the at least one two-dimensional image and a position and orientation of the three-dimensional model relative to the at least one two-dimensional image. Once the position and orientation of the at least one radiopaque object relative to the two-dimensional image is known and the position and orientation of the three-dimensional model relative to the at least one two-dimensional image is known, the image processing system 106 may use this information to determine the position and orientation of the at least one radiopaque object relative to the three-dimensional model. For example, the image processing system 106 may position and orient the at least one radiopaque object and the three-dimensional model relative to one another in a suitable reference frame, such as a virtual world reference frame in which the three-dimensional model defines the origin and direction of the reference frame coordinates. According to other examples, the virtual world reference frame may use an origin defined at a location other than the location of the three-dimensional model.

For the purposes of this disclosure, determining the position and orientation of a three-dimensional model of bony anatomy relative to a two-dimensional image may include determining a pose of the three-dimensional model with respect to the two-dimensional image that results in a projection of the three-dimensional model matching the bony anatomy shown in the two-dimensional image. Similarly, determining the position and orientation of a radiopaque object relative to a two-dimensional image may include determining a pose of a three-dimensional model of the radiopaque object with respect to the two-dimensional image that results in a projection of the model of the radiopaque object matching the radiopaque object shown in the two-dimensional image. The determination of the pose that results in a projection of the three-dimensional model of bony anatomy matching the representation of the bony anatomy shown in the two-dimensional image and the determination of the pose that results in a projection of the three-dimensional model of the radiopaque object matching the representation of the radiopaque object shown in the two-dimensional image may include accounting for imaging system characteristics. For example, X-ray system characteristics may be accounted for where the two-dimensional image is an X-ray image. X-ray system characteristics may include the geometry of the X-ray beam, the relative positions of the X-ray source and the X-ray detector, the calibration of the X-ray system, or other details.

The position and orientation of the three-dimensional model relative to the at least one two-dimensional image may be determined by identifying a projection of the three-dimensional model that aligns with the at least one two-dimensional image. A projection of the three-dimensional model that “aligns” with the at least one two-dimensional image is one in which the projection of the three-dimensional model sufficiently matches the bony anatomy shown in the two-dimensional image. For example, the image processing system 106 may compare a projection of the three-dimensional model with the bony anatomy shown in the two-dimensional image to determine the extent to which the projection overlaps with the bony anatomy shown in the two-dimensional image and may determine that the projection of the three-dimensional model aligns with the at least two-dimensional image if the projection completely overlaps or if the projection overlaps with the bony anatomy shown in the two-dimensional image within a threshold amount (e.g., a threshold of 95 percent overlap).

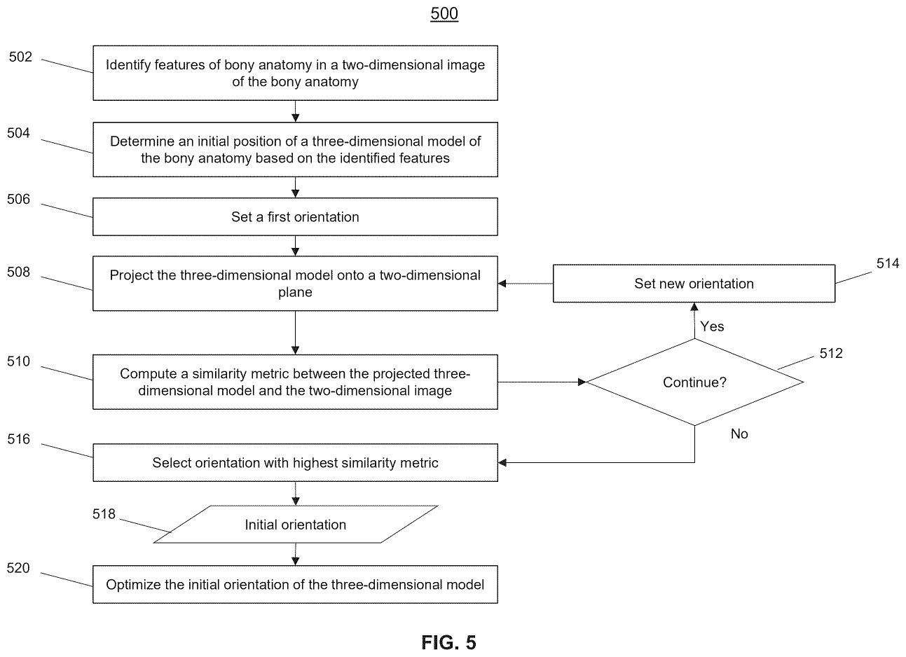

An exemplary method 500 for determining a projection of the three-dimensional image that aligns with the at least one two-dimensional image is illustrated in FIG. 5. Method 500 may be performed by one or more processors, such as one or more processors of image processing system 106 described above with reference to FIG. 1A.

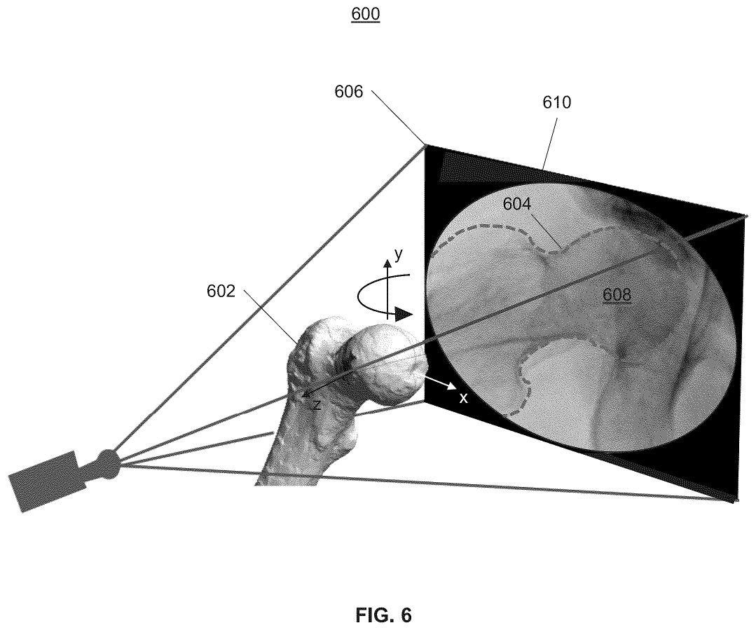

An example of method 500 related to debridement of a lesion (e.g., a cam pathology) on the head of a femur of a hip joint is shown in FIG. 6 to illustrate aspects of method 500. In the illustrated example, a three-dimensional model 602 models the upper portion of a particular patient's femur. A portion of bone may deviate from a target morphology and, as such, be identified for removal (e.g., via debridement). As used herein, “target morphology” may refer to any joint morphology that may be desired for a given subject. Target morphology can be based on the anatomy representative of any reference patient population, such as a normal patient population. For example, baseline data can be a model of a “normal” joint that is derived from studies of a healthy patient population and/or from a model generated based on measurements, computer simulations, calculations, etc.

As discussed further below, method 500 includes steps for determining a position and orientation of three-dimensional model 602 relative to the portion of the femur 608 captured in the two-dimensional image 610, such that a projection 604 of the model onto a two-dimensional plane 606 that corresponds with the plane of the two-dimensional image aligns with femur 608 in two-dimensional image 610. Conceptually speaking, the three-dimensional model 602 is manipulated according to the available degrees of freedom-such as translations in the x, y, and z directions, rotations about these axes, and scaling relative to the viewpoint of a simulated camera-until its projection 604 aligns sufficiently with the femur 608 in a two-dimensional image of the joint. The projection 604 may be determined to align sufficiently with the femur 608 if the projection 604 completely overlaps with the femur 608 shown in the two-dimensional image or if the projection 604 overlaps with the femur 608 shown in the two-dimensional image within a threshold amount (e.g., a threshold of 95 percent overlap).

Returning to FIG. 5, step 502 includes identifying features of bony anatomy in a two-dimensional image of the bony anatomy. The two-dimensional image analyzed may be the two-dimensional image received in step 202 of method 200. The features of bony anatomy identified in the two-dimensional image may include, for example, the position of the center of the femoral head in the two-dimensional image, the direction of the midline of the femoral neck, the radius of the femoral head, or any other suitable anatomical landmarks. Step 502 may be performed automatically by an image processing system (e.g., image processing system 106), semi-automatically by a combination of an image processing system and a user providing input, or manually by a user providing input. According to other aspects, method 500 may be performed without performing step 502.

At step 504, an initial position of the three-dimensional model may be determined based on the features identified in step 502. The center of the femoral head and the midline of the femoral neck in the image may be used to set an initial x and y position of the model. The center of the femoral head in the model may be positioned in the same location as the center of the femoral head in the two-dimensional image, and the midline of the femoral neck in the model may be positioned such that it extends in the same direction from the center of the femoral head as the midline of the femoral neck in the two-dimensional image. The radius of the femoral head may be used to determine an initial z position of the three-dimensional model. The z position of the three-dimensional model may be determined by calculating the distance between the three-dimensional model and a simulated camera that causes a projection of the three-dimensional model onto a two-dimensional plane to align with the two-dimensional image. The distance between the simulated camera and the pre-generated three-dimensional model may be calculated by first determining the radius of the femoral head in the two-dimensional image and the radius of the femoral head in the pre-generated three-dimensional model. The distance between the pre-generated three-dimensional model and the simulated camera that causes the radius of the three-dimensional femoral head shown in the three-dimensional model to become the radius of the two-dimensional femoral head shown in the two-dimensional image when projected into a two-dimensional plane may then be computed. According to other aspects, different anatomical landmarks may be used to determine the initial position of the 3D model.

Once the initial position of the three-dimensional model is determined, a first orientation of the three-dimensional model is set in step 506. In some examples, the first orientation may be set based on the likely orientation of the bone represented by the model during a surgical procedure. In other examples, the first orientation may be a predetermined orientation. For instance, an ultimate “initial orientation” (which may later be optimized) may be determined by performing a grid search that identifies a plurality of rotated positions of the three-dimensional model and computes a similarity metric between the two-dimensional image and a two-dimensional projection of the three-dimensional model for each position. In that case, the first orientation may be a predetermined rotation of the model, such as (−90,−90,−90).

Once the first orientation has been set, the three-dimensional model is projected onto a two-dimensional plane at step 508. The three-dimensional model may be projected onto the two-dimensional plane in the first orientation in order to visualize how closely the model in the first orientation aligns with the bone in the two-dimensional image.

A similarity metric is then computed between the projected three-dimensional model and the two-dimensional image at step 510. In some examples, the similarity metric used may be a cost function such as the intersection over union (IoU). IoU is a measure of overlap. To calculate IoU, the number of pixels that are shared between a pixel mask corresponding to the two-dimensional image and a pixel mask corresponding to the projection of the three-dimensional model onto the two-dimensional plane of the image is calculated. The total number of unique pixels contained in both masks combined is also calculated. IoU is then computed by dividing the number of shared pixels (the intersection) by the total number of unique pixels across both masks (the union). The possible values of IoU range from 0 (indicating there is no overlap between the pixel masks) to 1 (indicating a perfect overlap between the pixel masks).

Cost functions other than IoU may be used to evaluate alignment, such as reprojection error, iterative closest point (ICP) error, mutual information (MI), sum of squared distances (SSD), or normalized cross correlation (NCC). Optionally, the cost function used may have smooth gradients (e.g., IoU, MI, SSD, NCC) such that local minima do not interfere with locating the absolute minimum of the cost function (which may indicate the optimal orientation of the pre-generated three-dimensional model with respect to the two-dimensional image).

At step 512, a decision is made whether to continue to search for an initial orientation by setting a new orientation or to move on to the determination of the initial orientation based on the orientations already tested, such as by determining which orientation had the highest similarity metric when compared with the two-dimensional image. In some examples, if the similarity metric computed at step 510 for a given orientation exceeds a predetermined threshold value, the decision may be to stop iterating. In other examples, the decision of whether to continue to iterate is based on a predetermined criterion, such as whether a predetermined number of iterations have been completed. For instance, the initial orientation may be determined by performing a grid search in which a similarity metric is computed for multiple rotations of the three-dimensional model between the rotated positions (−90,−90,−90) and (90, 90, 90) by changing one variable at a time in 20-degree increments. The grid search may check the rotated positions (−90,−90,−90), (−90,−90, −70), (−90,−90,−50), and so on until reaching (90, 90, 90). The rotated position for which the similarity metric indicates the highest degree of similarity between the projection of the three-dimensional model and the two-dimensional image may be selected as the initial orientation of the 3D model. Thus, the decision may be to continue iterating until each 20-degree rotated position between (−90,−90,−90) and (90, 90, 90) has been checked. It should be appreciated that in other aspects, increments other than 20-degrees may be used.

If the decision at step 512 is to continue, a new orientation is set at step 514. As described above, the new orientation may be a predetermined rotated position between (−90,−90,−90) and (90, 90, 90). Once the new orientation is set, steps 508 and 510 may be repeated for the new orientation.

If the decision at step 512 is to stop iterating, the orientation with the highest similarity metric out of all orientations evaluated in the previous steps (i.e., the first orientation set in step 506 and all new orientations set in each iteration of step 514) may be selected at step 516. The selected orientation may be set as the initial orientation at step 518.

Once the initial orientation of the three-dimensional model is determined, the initial orientation is optimized at step 520 in order to complete registration of the three-dimensional model to the two-dimensional image. The orientation of the three-dimensional model may be optimized using a gradient descent algorithm. Optionally, the gradient descent algorithm may be the Adam algorithm. The gradient descent algorithm may compute the approximate gradient of the cost function (i.e., IoU) with respect to each variable. In the context of orientation, the variables include the six degrees of freedom: rotation in the x, y, and z directions and translation in the x, y, and z directions. The gradient descent algorithm may use the computed gradients to optimize each variable. The gradient descent algorithm may optimize each variable by using the finite difference method, partial differentiation, or any other suitable technique.

Using translation in the x-direction as an example, if the initial orientation of the three-dimensional model has an x-axis translation value of 10 mm, and the IoU for that value is 90, the gradient descent algorithm may compute the IoU for an x-axis translation value of 9 mm and an x-axis translation value of 11 mm. If the IoU for 9 mm is less than 90 and the IoU for 11 mm is greater than 90, the gradient descent algorithm may recognize that increasing the x-axis translation value above 10 mm improves IoU. The gradient descent algorithm may subsequently use an x-axis translation value greater than 10 mm in the next iteration. The subsequent x-axis translation value may be based on the computed gradient with respect to x-axis translation and the learning rate of the gradient descent algorithm. The learning rate may decay over time.

The gradient descent algorithm may continue to iteratively guess values for each variable using the techniques described above until the cost function for each variable is minimized or satisfies a predetermined criterion. For example, if IOU is used as the cost function, the gradient descent algorithm may continue to iteratively guess values for each variable until the IoU is equal to 1 (indicating a perfect alignment) or until the IoU exceeds a predetermined threshold (e.g., about 0.95). Alternatively, the gradient descent algorithm may continue to iterate for a predetermined amount of time or for a predetermined number of iterations. The end result of the iterative process is a determined three-dimensional model orientation that, when projected into the two-dimensional plane, aligns with the at least one two-dimensional image. According to other aspects, the initial orientation may be optimized using different techniques.

Returning to FIG. 2, a position and orientation of the at least one radiopaque object relative to the at least one two-dimensional image may also be determined at step 204 in order to determine the position and orientation of the at least one radiopaque object relative to the three-dimensional model. For example, continuing with the example of FIG. 3A, the at least one radiopaque object may be a radiopaque object with a known geometry, and the known geometry may be used to determine a position and orientation of the radiopaque object relative to the two-dimensional image. The known geometry of the radiopaque object may be in the form of a three-dimensional model of at least a portion of the radiopaque object, which may be retrieved by the image processing system 106 from the memory of the image processing system 106 or from a remote system 108. The position and orientation of the three-dimensional model of the radiopaque object relative to the radiopaque object in the two-dimensional image may be determined by manipulating the model according to the available degrees of freedom (e.g., translation in the x, y, and z directions, rotation about these axes, and scaling relative to the viewpoint of a simulated camera) such that a projection of the model onto a two-dimensional plane that corresponds with the plane of the two-dimensional image aligns with the at least one radiopaque object in the two-dimensional image. In some examples, the process for determining the position and orientation of the at least one radiopaque object with respect to the two-dimensional image may share one or more characteristics with the process for aligning a three-dimensional model of bony anatomy to a two-dimensional image of the same bony anatomy, as described above with reference to FIG. 5.

The position and orientation of the three-dimensional model relative to the at least one two-dimensional image may be combined with the position and orientation of the at least one radiopaque object relative to the two-dimensional image to obtain the position and orientation of the at least one radiopaque object relative to the three-dimensional model.

In some examples, determining the position and orientation of the radiopaque object relative to the three-dimensional model includes determining the position and orientation of the radiopaque object within a virtual world reference frame. The virtual world reference frame may be, for example, a coordinate system in which the three-dimensional model defines the origin and coordinate directions of the reference frame. The perspective in the virtual world reference frame from which a projection of the three-dimensional model aligns with what is shown in the two-dimensional image may define the position and orientation of the three-dimensional model relative to the two-dimensional image in the virtual world reference frame. Optionally, the two-dimensional image (e.g., a plane representing the two-dimensional image) may be represented within the virtual world reference frame based on the perspective in the virtual world reference frame from which a projection of the three-dimensional model aligns with what is shown in the two-dimensional image. According to various aspects, the two-dimensional imaging system used to capture the two-dimensional image may be an X-ray imaging system. One or both of the X-ray source and the X-ray detector may be positioned in the virtual world reference frame to determine the position and orientation of the three-dimensional model with respect to the two-dimensional image at which the projection of the three-dimensional model aligns with what is shown in the two-dimensional image while accounting for the X-ray imaging system characteristics. The position and orientation of the at least one radiopaque object relative to the at least one two-dimensional image may be combined with the position and orientation of the three-dimensional model relative to the two-dimensional image in the virtual world reference frame, resulting in the position and orientation of the at least one radiopaque object relative to the three-dimensional model in the virtual world reference frame.

At step 206, at least one endoscopic image that includes at least one feature associated with the at least one radiopaque object is received. The at least one endoscopic image may be captured by an endoscopic imager, such as endoscopic imager 112 of FIG. 1A, and provided to an image processing system, such as image processing system 106. The at least one endoscopic image may be captured intra-operatively, such as during a bone removal procedure, or pre-operatively. The endoscopic image(s) may be generated prior to performance of method 200 and retrieved from a suitable image storage system. Pre-generated endoscopic images may be captured when the patient is in the same position as they are when the two-dimensional image is captured so that the pre-generated endoscopic image captures the patient's anatomy in the same state it was in when the two-dimensional image was captured. According to other aspects, the pre-generated endoscopic image may be captured when the patient is in a different position compared to when the two-dimensional image was captured.

Optionally, multiple endoscopic images may be received, and the endoscopic images may depict the bony anatomy in a plurality of positions. Depicting the bony anatomy in a plurality of positions may be useful in providing multiple views of the bony anatomy from multiple different angles and/or showing relative positions of the bony anatomy relative to other anatomical features. To increase the amount of the bony anatomy that is viewable with the camera, the leg can be moved into a plurality of different positions. The plurality of positions may include predetermined positions, such as standard positions that are used during a surgical procedure. The standard positions of the leg include particular degrees or ranges of degrees of internal rotation, external rotation, flexion, extension, abduction, and/or adduction. During a hip surgery, the endoscopic images received at step 206 may include images of the bony anatomy in at least two of the standard positions in order to provide the surgeon with different views of the hip joint. Alternatively, the received endoscopic images may include images of the bony anatomy in a combination of standard and non-standard positions. For example, the received endoscopic images may include images of the bony anatomy in at least one of the standard positions and at least one non-standard position. Alternatively, the received endoscopic images may include images of the bony anatomy in a plurality of non-standard positions. Alternatively, the received endoscopic images may include images of the bony anatomy in the same position.

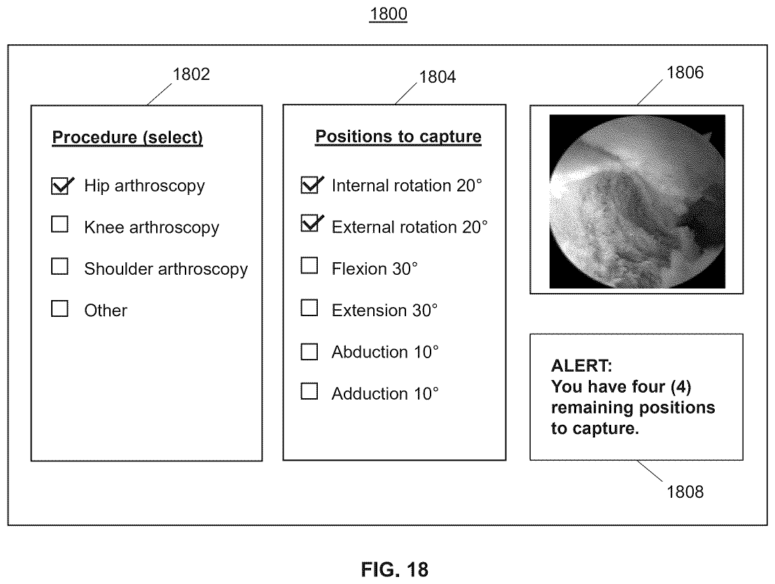

A surgeon may be provided with guidance to position the bony anatomy in a plurality of different positions. FIG. 18 illustrates an exemplary graphical user interface 1800 for guiding a surgeon to place bony anatomy in a plurality of positions. Graphical user interface 1800 may be displayed on display(s) 120, and a surgeon may interact with graphical user interface 1800 via user control device 122. Graphical user interface 1800 includes a procedure type panel 1802 that includes a list of surgical procedures (e.g., hip arthroscopy, knee arthroscopy, shoulder arthroscopy, other). A surgeon may indicate the type of procedure, for example by operating user control device 122 to check a checkbox corresponding to the appropriate surgical procedure (e.g., via a button press). The illustrated checkboxes are merely exemplary, and it should be understood that a surgeon can indicate a procedure type using any number of graphical user interface options. For example, the surgeon may indicate which procedure is ongoing by selecting a surgical procedure from a drop-down menu or toggling on a slider corresponding to a surgical procedure.

Based on the procedure selected by the surgeon in procedure panel 1802, a position panel 1804 may be populated with a list of positions. The positions listed in position panel 1804 correspond to the procedure type selected on procedure panel 1802. For example, as shown in FIG. 18, when the selected surgical procedure is a hip arthroscopy, position panel 1804 displays a list of leg positions used in hip arthroscopy surgery, which include various degrees of internal rotation, external rotation, flexion, extension, abduction, and adduction. Each position listed in position panel 1804 may include a corresponding check box. A surgeon may operate user control device 122 to check a box corresponding to a position once endoscopic video of the bony anatomy in that position has been captured.

Optionally, graphical user interface 1800 may display live endoscopic video 1806, such that a surgeon can view the endoscopic video side-by-side with the positioning guidance (e.g., to ensure that adequate views of the bony anatomy in the various positions are captured). Graphical user interface 1800 may also include an alert panel 1808. Alert panel 1808 may indicate to the surgeon a number of remaining positions in which to capture endoscopic video of the bony anatomy. For example, in FIG. 18, alert panel 1808 indicates that there are four remaining bony anatomy positions to capture in the endoscopic video.

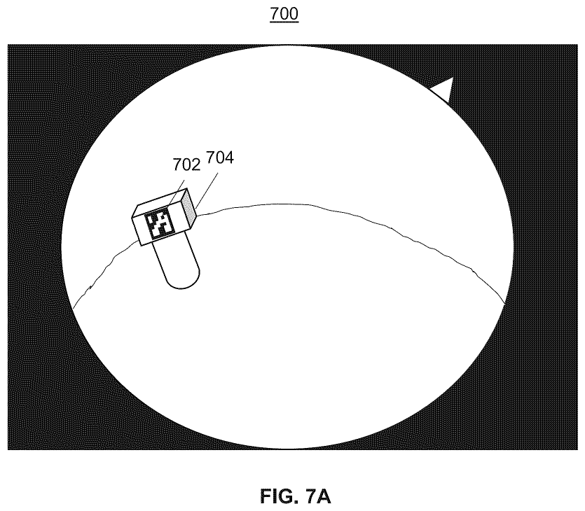

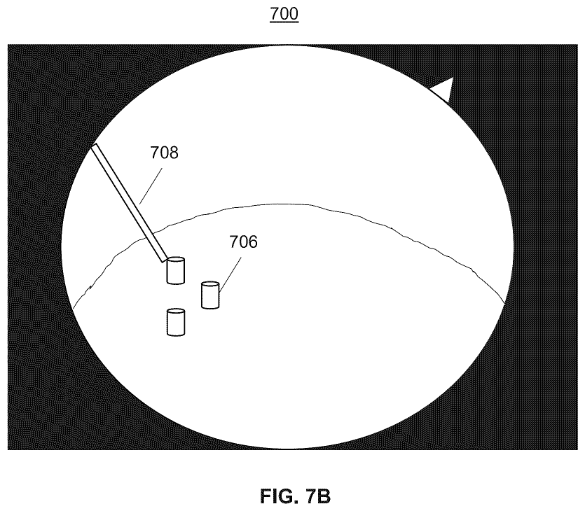

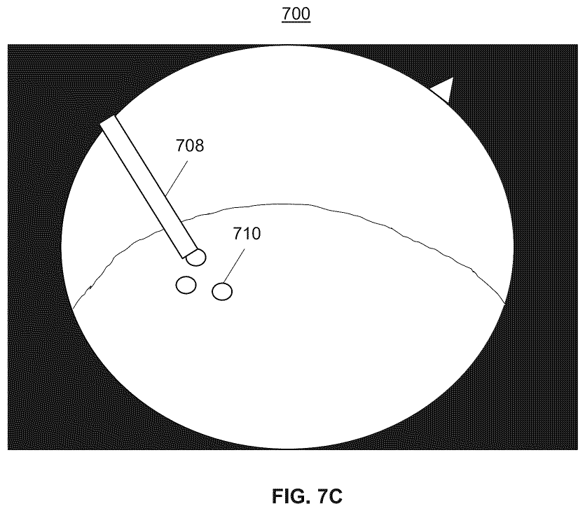

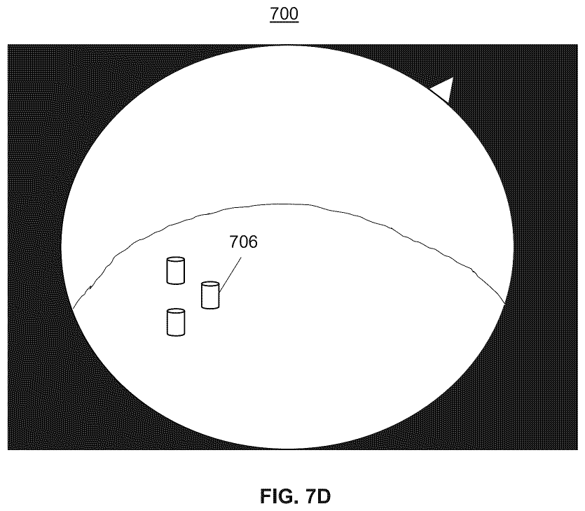

The at least one endoscopic image includes at least one feature associated with the at least one radiopaque object. In some examples, the at least one feature associated with the at least one radiopaque object may be the shape of the at least one radiopaque object itself. For example, the at least one radiopaque object may have a sufficiently unique geometry such that the shape of the at least one radiopaque object is recognizable in the endoscopic view. In some examples, the at least one feature associated with the at least one radiopaque object may be a fiducial marker. The fiducial marker may be an optically visible marker that is visible in endoscopic images. For instance, continuing with the example of FIG. 3A in which the at least one radiopaque object is a radiopaque object with a known geometry, the at least one feature associated with the at least one radiopaque object may be a fiducial marker attached to or printed on the radiopaque object with the known geometry. FIG. 7A illustrates an example of an endoscopic image 700 that includes a fiducial marker 702 that has a predetermined pattern and is associated with a radiopaque object 704. The fiducial marker 702 shown in the example of FIG. 7A is an ArUco, but fiducial marker 702 may also be a QR code, RF marking, or any other type of predetermined pattern. Fiducial marker 702 may be integral with radiopaque object 704 (e.g., printed on radiopaque object 704) or may be part of a separate structure that is affixed to or otherwise associated with radiopaque object 704.

The at least one radiopaque object may be a radiopaque tool, such as a tool for positioning objects on or in the bone or a tool for burring depressions in the bone, as shown in FIG. 3B, and the at least one feature associated with the radiopaque tool may be a plurality of objects positioned by the tool. FIG. 7B illustrates an exemplary endoscopic image 700 that includes a tool 708 used to position a plurality of objects 706. The plurality of objects may be pins, beads, gel drops, pieces of bone wax, or any optically visible objects. The plurality of objects may be non-radiopaque objects or radiopaque objects. Regardless of whether radiopaque or non-radiopaque objects are used, the objects include optically visible portions that can be seen in the endoscopic image 700. Alternatively, tool 708 may be used to create a plurality of depressions in the bone. FIG. 7C shows an exemplary endoscopic image 700 that includes tool 708 and a plurality of depressions 710 burred in the bony anatomy by the tool. Tool 708 may also be used to create markings in the bony anatomy other than a plurality of depressions (e.g., RF markings).

The at least one radiopaque object may be a plurality of radiopaque objects, as shown in FIG. 3C, and the at least one feature associated with the radiopaque objects may be an optically visible portion of the plurality of objects. FIG. 7D shows an exemplary endoscopic image 700 illustrating a plurality of small objects 706 inserted into bony anatomy. The plurality of small objects 706 shown in the endoscopic image may include the optically visible portions of the plurality of radiopaque objects.

At step 208, a position and orientation of the at least one feature associated with the at least one radiopaque object relative to the at least one endoscopic image is determined. For the purposes of this disclosure, determining the position and orientation of the at least one feature associated with the at least one radiopaque object relative to the at least one endoscopic image may include determining a pose of the at least one feature with respect to the endoscopic image that results in a projection of the at least one feature matching the representation of the at least one feature shown in the endoscopic image. As discussed in greater detail below, determining the pose of the at least one feature with respect to the endoscopic image that results in a projection of the at least one feature matching the representation of the at least one feature shown in the endoscopic image may involve accounting for the characteristics of the optics of the endoscopic imager used to capture the endoscopic image.

The position and orientation of the at least one feature associated with the at least one radiopaque object relative to the at least one endoscopic image may be determined by analyzing the at least one endoscopic image (e.g., by one or more processors of image processing system 106). Continuing with the example of FIG. 3A in which the at least one radiopaque object is a radiopaque object with a known geometry and the at least one feature associated with the at least one radiopaque object is a fiducial marker, a position and orientation of the fiducial marker relative to the at least one endoscopic image may be determined by analyzing the at least one endoscopic image to identify a location of the fiducial marker within the image. The predetermined pattern of the fiducial marker may be known along with the relationship between the fiducial marker and the radiopaque object. Image processing system 106 may identify characteristics of the fiducial marker within the at least one endoscopic image (e.g., the relative locations and/or shapes of various features of the fiducial marker). The image processing system 106 may compare the characteristics of the fiducial marker in the image with the predetermined pattern of the fiducial marker to determine the position and orientation of the fiducial marker in the endoscopic image. Image processing system 106 may use image analysis techniques (e.g., object detection models) or machine learning techniques (e.g., deep learning techniques) to determine the position and orientation of the fiducial marker relative to the endoscopic image. Optionally, predetermined characteristics of the optics of the endoscopic imager used to capture the endoscopic image may be used to determine the position and orientation of the fiducial marker relative to the endoscopic image. For example, the location of the fiducial marker within the endoscopic image may be determined using any of the techniques described above (e.g., object detection models, and/or machine learning models). Characteristics of the optics (e.g., the focal length) of the endoscopic imager used to capture the endoscopic image may be predetermined, such as from calibration of the endoscopic imager. Based on the determined location of the fiducial marker within the endoscopic image and the characteristics of the optics of the endoscopic imager used to capture the endoscopic image, the position and orientation of the fiducial marker in three-dimensional space that results in a projection of the fiducial marker matching what is shown in the endoscopic image may be determined.

In some examples, determining the position and orientation of the at least one feature relative to the at least one endoscopic image includes determining the position and orientation of the endoscopic imager used to capture the at least one endoscopic image within the virtual world reference frame. For example, a perspective of the endoscopic imager can be determined based on the at least one feature shown in the at least one endoscopic image, the known geometry of the at least one feature, and one or more characteristics of the optics of the endoscopic imager. This perspective can be combined with the position and orientation of the at least one feature within the virtual world reference frame (which may be determined based on the position and orientation of the at least one radiopaque object in the virtual world reference frame determined at step 204 and a known relationship between the at least one radiopaque object and the at least one feature) to determine the position and orientation of the endoscopic imager in the virtual world reference frame.

At step 210, an alignment of the three-dimensional model relative to the at least one endoscopic image is determined (e.g., by one or more processors of image processing system 106). The three-dimensional model is “aligned” relative to the at least one endoscopic image when a projection of the three-dimensional model matches the bony anatomy shown in the at least one endoscopic image. The position and orientation of the at least one radiopaque object relative to the three-dimensional model has been determined from step 204. The position and orientation of the at least one feature associated with the at least one radiopaque object relative to the at least one endoscopic image has been determined from step 208. The position and orientation of the at least one radiopaque object relative to the at least one endoscopic image may then be determined based on a known relationship between the at least one radiopaque object and the at least one feature associated with the at least one radiopaque object. Continuing with the example of FIG. 3A, the at least one radiopaque object may be a radiopaque object with a known geometry, and the associated feature may be a fiducial marker that is attached to or printed on the radiopaque object. The position and orientation of the fiducial marker relative to the radiopaque object may be known. For instance, a three-dimensional model of the radiopaque object stored in a memory of image processing system 106 or remote system 108 may indicate which surface of the radiopaque object the fiducial marker is attached to or printed on. The image processing system 106 may use the known relationship between the fiducial marker and the radiopaque object to determine the position and orientation of the radiopaque object relative to the endoscopic image and/or the position and orientation of the fiducial marker relative to the three-dimensional model. The position and orientation of the object relative to the three-dimensional model determined at step 204 may be combined with the position and orientation of the object relative to the fiducial marker to determine a position and orientation of the fiducial marker relative to the three-dimensional model or a position and orientation of the object relative to the endoscopic image. The position and orientation of the fiducial marker relative to the three-dimensional model or the position and orientation of the object relative to the endoscopic image may be combined with the position and orientation of the fiducial marker relative to the at least one endoscopic image to achieve the position and orientation of the three-dimensional model relative to the at least one endoscopic image.

At step 212, at least a portion of the three-dimensional model is displayed based on the alignment of the three-dimensional model relative to the at least one endoscopic image. The three-dimensional model may be displayed to a surgeon to guide bone removal, for example via display 120 described above with reference to FIG. 1A.

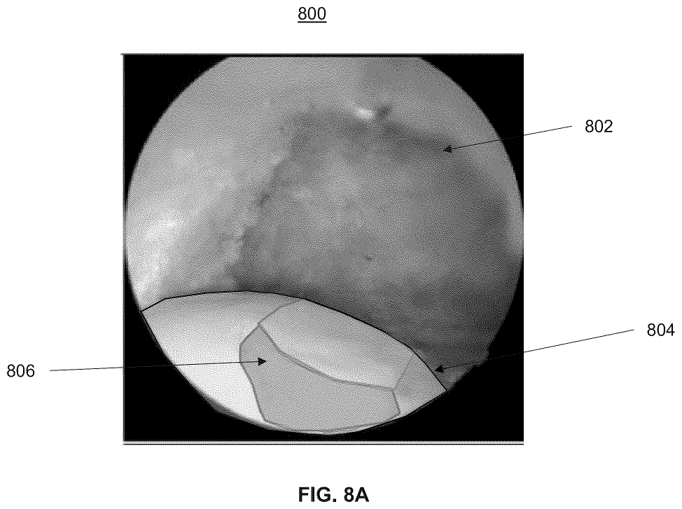

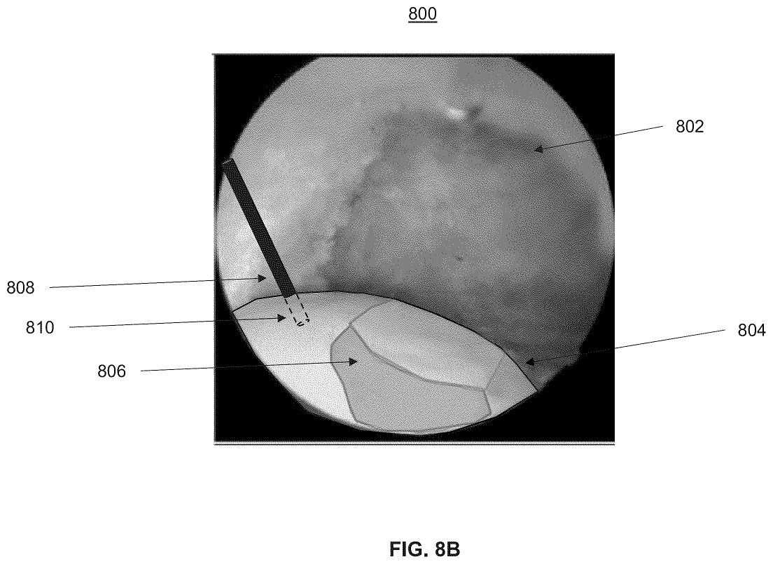

The at least a portion of the three-dimensional model may be displayed in various manners. For example, the at least a portion of the three-dimensional model may be displayed as an overlay on the endoscopic image. An exemplary overlay image 800 is shown in FIG. 8A. FIG. 8A shows a portion of a three-dimensional model 804 overlaid on an endoscopic image 802. In the overlay, three-dimensional model 804 is aligned with endoscopic image 802, such that a projection of three-dimensional model 804 into two dimensions matches the bony anatomy shown in endoscopic image 802. For example, when aligned with endoscopic image 802, the three-dimensional model 804 is positioned such that the perspective and scale of the displayed three-dimensional model 804 matches with the bony anatomy shown in endoscopic image 802. Optionally, three-dimensional model 804 may extend beyond the boundaries of the endoscopic image 802, such that anatomical structures outside of the endoscopic field of view are visible. Three-dimensional model 804 includes a heat map 806 covering a portion of the bony anatomy that deviates from a target bone morphology. Heat map 806 may serve as a plan for a surgical procedure that indicates to the surgeon where and how much bone should be removed to achieve the target morphology. Heat map 806 may be color-coded according to the planned depth of bone removal. For example, as shown in FIG. 8A, heat map 806 includes a teal blue portion corresponding to a first depth of bone removal and a dark blue portion corresponding to a different, second depth of bone removal. Alternatively, or additionally, an outline of the planned bone removal area, a contour map indicating amounts of bone removal, or any other suitable visual aid may be used to represent planned bone removal. Optionally, three-dimensional model 804 (and heat map 806) may be at least partially transparent, such that the bony anatomy in the endoscopic image is visible through the overlay of three-dimensional model 804 and heat map 806. The heat map 806 (or any other indication of planned bone removal) may also be toggled on and off entirely by a user in accordance with the user's preferences. Optionally, heat map 806 may be directly overlaid onto endoscopic image 802. In other words, heat map 806 may be overlaid onto endoscopic image 802 without the bone of three-dimensional model 804.

In some examples, the endoscopic image 802 may capture a surgical instrument 808 (e.g., a bone removal tool such as a bur, shaver, drill, saw, etc.), as shown in FIG. 8B. Surgical instrument 808 may be located in a portion of endoscopic image 802 that is overlaid with three-dimensional model 804. For example, the distal portion 810 of surgical instrument 808 is located in the region of endoscopic image 802 covered by three-dimensional model 804. As discussed above, three-dimensional model 804 may be partially transparent. Thus, the portion of surgical instrument 808 covered by three-dimensional model 804 (e.g., distal portion 810) may be visible through the partially transparent three-dimensional model 804. In implementations where surgical instrument 808 is a bone removal tool, this may allow a user to visualize where bone is being removed. In some aspects, an indication of planned bone removal (e.g., heat map 806) may be displayed on top of at least a portion of surgical instrument 808. The indication of planned bone removal may be opaque, such that the surgical instrument 808 does not obscure the indication of planned bone removal. Alternatively, the indication of planned bone removal may be at least partially transparent, such that surgical instrument 808 is at least partially visible through the indication of planned bone removal. In some examples, surgical instrument 808 may be identified in the endoscopic image (e.g., by one or more machine learning models). The one or more machine learning models may determine that a portion of surgical instrument 808 overlaps with the area of endoscopic image 802 covered by three-dimensional model 804 (and, optionally, heat map 806). Image processing system 106 may indicate the portion of the surgical instrument 808 that overlaps with three-dimensional model 804 and/or heat map 806 using coloring, shading, or patterns. For example, in FIG. 8B, distal portion 810 represents the portion of the surgical instrument 808 that overlaps with three-dimensional model 804 (the overlapping portion is schematically represented using dashed lines). In implementations involving a bone removal instrument, the one or more machine learning models may identify the bone removing feature of the instrument (e.g., distal portion 810 of surgical instrument 808) in the endoscopic image 802. Image processing system 106 may generate a visualization in which the three-dimensional model 804 is overlaid on the entirety of the endoscopic image 802 except for a region corresponding to distal portion 810, such that a user can visualize where bone is being removed. Similarly, if the surgical instrument 808 overlaps with the heat map 806 (or other indication of planned bone removal), image processing system 106 may generate a visualization in which the heat map 806 is not displayed in the region corresponding to the surgical instrument 808 in order to allow the user to clearly visualize the location of the surgical instrument 808 without the heat map 806 obstructing their view.

In some examples, a visualization may include a representation of a surgical instrument (e.g., a three-dimensional model of the surgical instrument). In aspects where the visualization includes an overlay of three-dimensional model 804 on endoscopic image 802, the representation of the surgical instrument may be displayed in a location relative to the portion of three-dimensional model 804 that corresponds to the location of surgical instrument 808 relative to the bony anatomy in endoscopic image 802. Three-dimensional model 804 may include a heat map 806, and heat map 806 may not be displayed in an area corresponding to the surgical instrument (e.g., an area corresponding to a bone removal feature of the surgical instrument), such that the representation of the surgical instrument (or the bone removal feature thereof) is not obscured by heat map 806. Three-dimensional model 804 may optionally be displayed separately from endoscopic image 802. In aspects where the visualization includes a representation of a surgical instrument and an overlay of heat map 806 directly on endoscopic image 802 (without the rest of three-dimensional model 804), heat map 806 may not be displayed in the area corresponding to the representation of the surgical instrument so as to ensure that the representation of the surgical instrument remains visible to a surgeon.

Alternatively, heat map 806 may be displayed in the area corresponding to the representation of the surgical instrument, and the representation of the surgical instrument may be adjusted by a user so that heat map 806 is visible or obscured. For example, the representation of the surgical instrument may be opaque, such that heat map 806 is obscured by the representation of the surgical instrument. Alternatively, the representation of the surgical instrument may be at least partially transparent, such that heat map 806 is at least partially visible underneath the representation of the surgical instrument. The transparency of the representation of the surgical instrument may be selectively adjusted by a user. Optionally, only a silhouette or outline of the surgical instrument may be displayed to ensure that heat map 806 is visible. Optionally, the representation of the surgical instrument may be toggled on and off entirely. It should be understood that the representation of the surgical instrument can be displayed in the above-described manner in any aspects that display a representation of a surgical instrument with respect to a heat map (e.g., a three-dimensional model with a heat map, an overlay of a three-dimensional model with a heat map on a two-dimensional image, an overlay of a heat map directly on a two-dimensional image, etc.).

Optionally, one or more machine learning models may be used to ensure that the three-dimensional model 804 and any indication of planned bone removal (e.g., heat map 806) is displayed in an accurate position relative to the bony anatomy pictured in the endoscopic image 802. For example, one or more machine learning models may be configured to identify tissue (e.g., soft tissue or bone), anatomical landmarks, and/or abnormalities shown in the endoscopic image 802. The same or different machine learning model(s) may determine an appropriate placement of the indication of planned bone removal relative to the endoscopic image 802 based on the identified tissue, landmarks, and/or abnormalities (e.g., the machine learning model(s) may align a region of planned bone removal in the indication of planned bone removal with an identified abnormality). Alternatively or additionally, the one or more machine learning models may identify other objects in the endoscopic image 802 to ensure accurate positioning of the indication of planned bone removal. The machine learning model(s) may be configured to identify one or more surgical instruments in the endoscopic image 802 and determine an appropriate placement of the indication of planned bone removal relative to the endoscopic image 802 based on the location of the surgical instrument(s). For example, the machine learning model(s) may determine the presence of a bone removal portion of a bone removal instrument and determine that a portion of the indication of planned bone removal should align with the bone removal portion of the bone removal instrument.

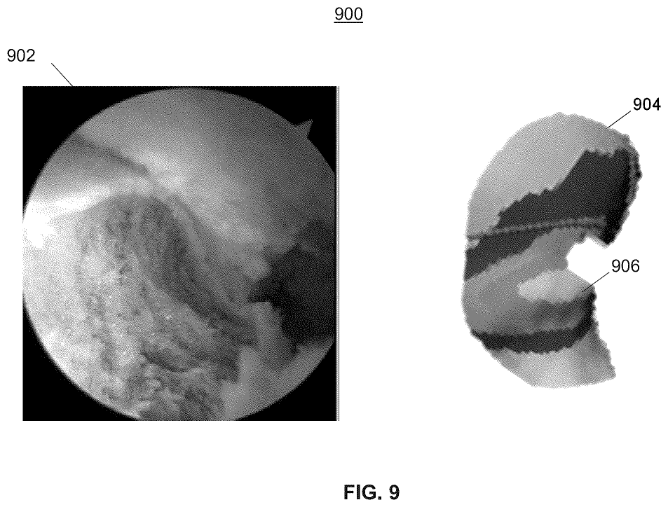

In some aspects, at least a portion of the three-dimensional model may be displayed simultaneously with but separately from the at least one endoscopic image. For example, as illustrated in FIG. 9, a visualization 900 may include an endoscopic image 902 displayed next to a corresponding portion of a three-dimensional model 904 that aligns with endoscopic image 902. Endoscopic image 902 and three-dimensional model 904 are aligned when a projection of three-dimensional model 904 into two-dimensional space matches the bony anatomy that is shown in endoscopic image 902. For example, the displayed three-dimensional model 904 may have the same scale as the bony anatomy shown in endoscopic image 902 and may be shown from the same perspective as the bony anatomy shown in endoscopic image 902. As shown in FIG. 9, the three-dimensional model 904 may optionally include an indication of planned bone removal 906. The indication of planned bone removal 906 may be toggled on and off by a user in accordance with the user's preferences.

Optionally, a representation of a surgical instrument may be displayed with respect to the three-dimensional model. The representation of the surgical instrument may be displayed in a location relative to the portion of the three-dimensional model that corresponds to the location of a surgical instrument relative to the bony anatomy in the endoscopic image. The representation of the surgical instrument may be opaque or may be at least partially transparent. The transparency of the representation of surgical instrument may be adjusted by a user. The representation of the surgical instrument may also be toggled on and off entirely by a user. In some implementations, the representation of the surgical instrument may include only an outline or silhouette of the surgical instrument.

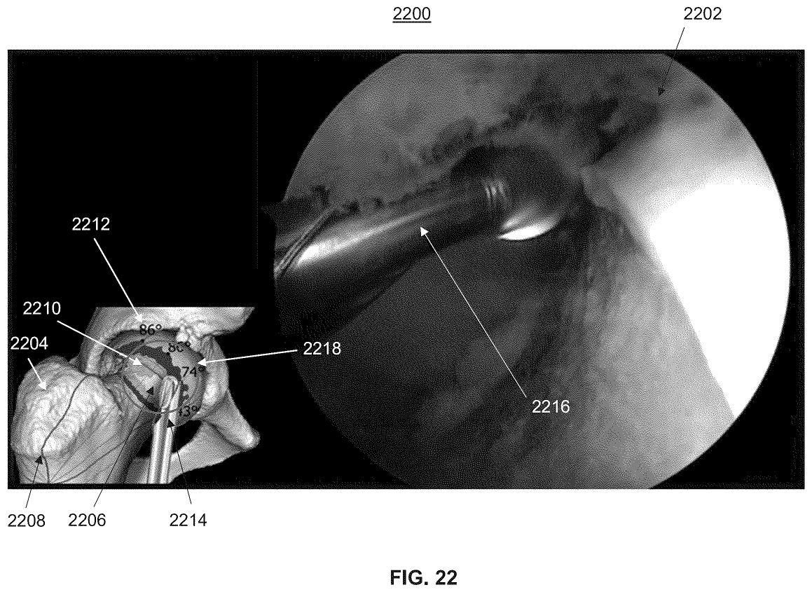

As noted above, the displayed endoscopic image 902 and three-dimensional model 904 may be shown at the same scale. Alternatively, the endoscopic image 902 and three-dimensional model 904 may be shown at different scales. For example, the three-dimensional model and endoscopic image may be displayed using “picture-in-picture” techniques, in which the endoscopic image is displayed prominently, and the three-dimensional model is displayed in a smaller window overlaid on a portion of the endoscopic image. FIG. 22 illustrates a visualization 2200 in which endoscopic video 2202 is displayed prominently in a first portion of visualization 2200, and a portion of the three-dimensional model 2204 corresponding to the displayed endoscopic video 2202 is displayed in a second, less prominent portion of visualization 2200. The portion of the three-dimensional model 2204 may include an indication of planned bone removal, such as heat map 2206. Alternatively, the portion of the three-dimensional model 2204 may be displayed without heat map 2206.

Optionally, visualization 2200 may indicate which region of the displayed portion of three-dimensional model 2204 is being shown in endoscopic image 2202. For example, as shown in FIG. 22, a circle 2218 encloses the region of three-dimensional model 2204 that corresponds to endoscopic image 2202. Alternatively, the region of three-dimensional model 2204 corresponding to what is shown in endoscopic image 2202 may be indicated by highlighting the region of three-dimensional model 2204 being shown and/or by graying out the region of three-dimensional model 2204 that is not being shown. Alternatively, or additionally, the region of three-dimensional model 2204 being shown may be displayed using a different colorization (e.g., by brightening the region being viewed, increasing the contrast in the region being viewed, etc.).

Optionally, the portion of the three-dimensional model 2204 may include one or more clock-face lines 2208. As is known in the art, clock-face lines are useful for surgeons to identify positions within a joint (e.g., within a hip joint, clock-face lines may help a surgeon identify rotational positions about the femoral head or the acetabular cup). The portion of the three-dimensional model 2204 may also include one or more Alpha Angle lines 2210. An Alpha Angle line 2210 may represent the set of circumferential locations where the bony anatomy first extends outside of a best-fit sphere (or a plurality of best-fit circles) around the femoral head. The portion of the three-dimensional model 2204 may also include one or more Alpha Angle points 2212, which may indicate the location of the start of a given pathology relative to a predetermined clock-face position.

As discussed above, a representation of a surgical instrument 2214 may be displayed with respect to the portion of the three-dimensional model 2204. The representation of the surgical instrument 2214 may be a representation of surgical instrument 2216, which is shown in endoscopic image 2202. The representation of the surgical instrument 2214 may be displayed in a location relative to the portion of the three-dimensional model 2204 that corresponds to the location of surgical instrument 2216 relative to the bony anatomy in endoscopic image 2202. The representation of the surgical instrument 2214 may be opaque, as shown in FIG. 22, or may be at least partially transparent. The user may selectively adjust the transparency of the representation of the surgical instrument 2214. The representation of the surgical instrument 2214 may be toggled on and off by a user.

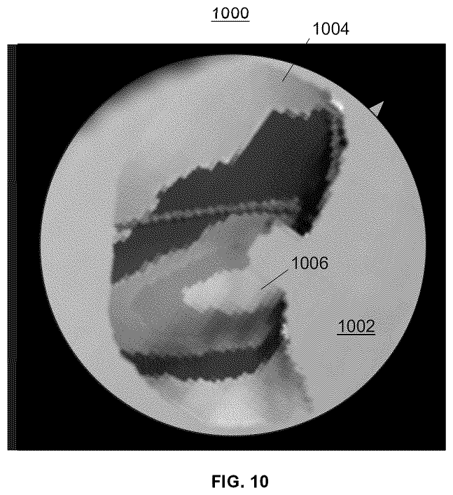

In some aspects, the at least a portion of the three-dimensional model may be displayed as a simulated endoscopic image that includes the portion of the three-dimensional model that corresponds to the bony anatomy captured in the endoscopic image. The simulated endoscopic image may be displayed based on the determined alignment between the three-dimensional model and the at least one endoscopic image. The three-dimensional model and the endoscopic image are aligned when a projection of the three-dimensional model matches the bony anatomy that is shown in the at least one endoscopic image. The simulated endoscopic image may, for example, show the same anatomy, from the same perspective, as the anatomy represented in the at least one endoscopic image. An exemplary simulated endoscopic image 1000 is illustrated in FIG. 10. Simulated endoscopic image 1000 shows a simulated field of view 1002 of an endoscope. The simulated endoscopic image 1000 includes only the portion of the three-dimensional model 1004 that corresponds to what is shown in the endoscopic image being simulated. Optionally, the simulated endoscopic image 1000 includes more of the three-dimensional model 1004 beyond what is shown in the endoscopic image being simulated (e.g., the three-dimensional model may extend beyond the boundaries of what is shown in the simulated endoscopic field of view). Display of the simulated endoscopic image 1000 may be advantageous in providing the surgeon with the portion of the three-dimensional model 1004 that corresponds to what is shown in the endoscopic image but without obscuring the endoscopic image. As shown in FIG. 10, three-dimensional model 1004 may include an indication of planned bone removal 1006. The indication of planned bone removal 1006 may be in the form of a color-coded heat map, where different colors indicate differing depths of bone removal. The indication of planned bone removal 1006 may be toggled on and off by a user in accordance with the user's preferences.

Optionally, the simulated endoscopic image 1000 may be shown in association with a real endoscopic image using picture-in-picture techniques. For example, similar to what is shown in FIG. 22, the real endoscopic image may be displayed prominently, and simulated endoscopic image 1000 may be displayed in a smaller window overlaid on a portion of the real endoscopic image. In other examples, the simulated endoscopic image 1000 may be displayed prominently, and the real endoscopic image may be displayed in a smaller window overlaid on a portion of simulated endoscopic image 1000.

Optionally, the simulated endoscopic image 1000 may include a representation of a surgical instrument. The representation of the surgical instrument may be displayed in a location that corresponds to the location of a surgical instrument relative to the bony anatomy in the real endoscopic image. The representation of the surgical instrument may be opaque or may be at least partially transparent. The transparency of the representation of surgical instrument may be adjusted by a user. The representation of the surgical instrument may also be toggled on and off entirely by a user. In some implementations, the representation of the surgical instrument may include only an outline or silhouette of the surgical instrument. As discussed above with respect to FIG. 8B, if the surgical instrument overlaps with the indication of planned bone removal 1006, image processing system 106 may generate a visualization in which the indication of planned bone removal 1006 is not displayed in the region corresponding to the surgical instrument in order to allow the user to clearly visualize the location of the surgical instrument without the indication of planned bone removal obstructing their view.

The simulated endoscopic image 1000 may provide a simplified view to a user. For instance, the simulated endoscopic image 1000 may omit unnecessary tissues and/or blockages from view. The simulated endoscopic image 1000 may also result in enhanced image display (e.g., by removing jitter from the displayed image). Furthermore, the simulated endoscopic image 1000 may be shown in a non-distorted manner as compared to a regular, non-simulated endoscopic image, which may appear distorted.

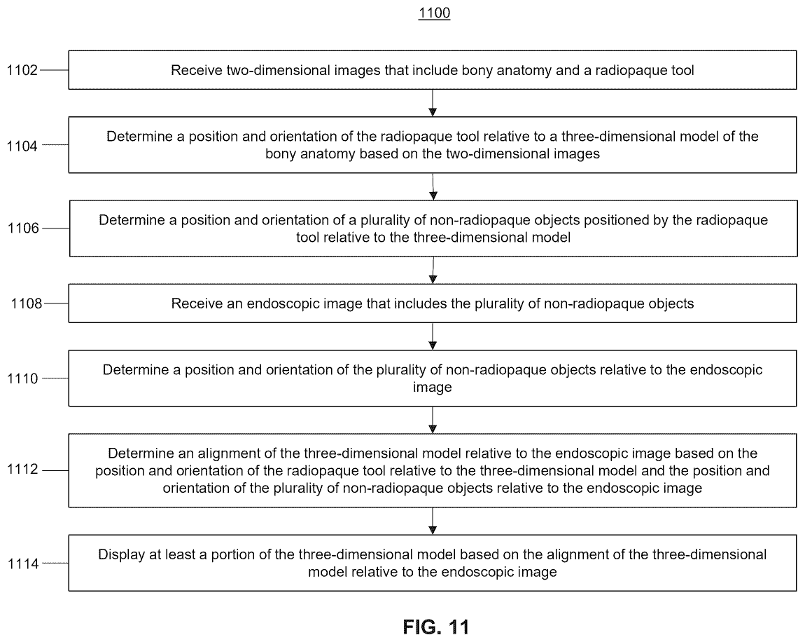

Aspects of method 200 have been described above using an example in which the at least one radiopaque object is a radiopaque object with a known geometry and the at least one feature associated with the at least one radiopaque object is a fiducial marker attached to or printed on the radiopaque object. As noted above, method 200 may be performed using various different radiopaque objects and/or features associated with the radiopaque objects. For example, the at least one radiopaque object of method 200 may be a radiopaque tool, as discussed above with reference to FIG. 3B, and the at least one feature associated with the at least one radiopaque object may be a plurality of non-radiopaque objects positioned by the radiopaque tool. Method 1100 of FIG. 11 illustrates an exemplary implementation of method 200 in which a radiopaque tool and a plurality of non-radiopaque objects are used to determine an alignment of a three-dimensional model relative to at least one endoscopic image. Method 1100 may be performed by one or more processors of image processing system 106 or any other suitable computing system. Aspects of method 1100 are described above with respect to method 200 and are not repeated below for simplicity.

At step 1102, two-dimensional images that includes bony anatomy and a radiopaque tool are received. As described above with reference to step 202 of FIG. 2, the two-dimensional images may be fluoroscopic images received, for example, by image processing system 106 from two-dimensional imaging system 110 of FIG. 1A. The two-dimensional images may include the portion of the bone that is being or will be surgically treated, as well as surrounding portions of the bone that enable the surgeon to compare what is shown in the two-dimensional image to what the surgeon sees endoscopically.

The two-dimensional images include a radiopaque tool. FIG. 3B shows an exemplary two-dimensional image 300 that includes bony anatomy 302 and a radiopaque tool 306. The radiopaque tool 306 itself may be radiopaque, as shown in FIG. 3B, or the radiopaque tool may include one or more radiopaque fiducials (e.g., radiopaque beads) affixed to an otherwise non-radiopaque tool. As shown in FIG. 3B, radiopaque tool 306 may be used to position a plurality of non-radiopaque objects relative to the bony anatomy 302 that do not appear in two-dimensional image 300. However, it should be understood that objects positioned by radiopaque tool 306 may also be radiopaque.

Multiple two-dimensional images are received at step 1102 because multiple non-radiopaque objects may be positioned using the radiopaque tool. As discussed in greater detail below, the multiple non-radiopaque objects may be positioned in arbitrary locations by the surgeon. Thus, image processing system 106 may not possess predetermined knowledge of the position and orientation of the non-radiopaque objects relative to the three-dimensional model that is required to align the three-dimensional model with an endoscopic image that includes the non-radiopaque objects. Accordingly, to determine the position and orientation of the non-radiopaque objects relative to the three-dimensional model, a two-dimensional image of the radiopaque tool may be captured when the tip of the radiopaque tool is positioning each non-radiopaque object. The position and orientation of the radiopaque tool relative to the three-dimensional model may be determined based on each two-dimensional image in step 1104 discussed below. Based on the position and orientation of the tip of the tool relative to the three-dimensional model in each two-dimensional image, the position and orientation of each non-radiopaque object relative to the three-dimensional model may be determined by one or more processors of image processing system 106, since each non-radiopaque object is positioned by the radiopaque tool.

At step 1104, a position and orientation of the radiopaque tool relative to a three-dimensional model of the bony anatomy is determined based on the two-dimensional images. Step 1104 may share one or more characteristics with step 204 of FIG. 2. For example, a position and orientation of the radiopaque tool relative to each two-dimensional image may be determined by aligning a projection of a three-dimensional model of the radiopaque tool stored in a memory of image processing system 106 or remote system 108 with the respective two-dimensional image. A position and orientation of the three-dimensional model of the bony anatomy may also be aligned with the two-dimensional images by aligning a projection of the three-dimensional model into two-dimensional space with the two-dimensional images. The position and orientation of the radiopaque tool relative to the three-dimensional model of the bony anatomy may then be determined based on the position and orientation of the radiopaque tool relative to the two-dimensional images and the position and orientation of the three-dimensional model relative to the two-dimensional images. As used herein, determining the position and orientation of the three-dimensional model of the radiopaque tool and the three-dimensional model of bony anatomy relative to the two-dimensional images may include determining a pose of the respective three-dimensional model with respect to the two-dimensional images that results in a projection of the respective three-dimensional model that matches what is shown in the two-dimensional images. The two-dimensional images may be X-ray images, and determining the pose of the respective model with respect to the two-dimensional images may include accounting for X-ray system characteristics, as described above.

Determining the position and orientation of the radiopaque tool relative to the three-dimensional model may include determining the position and orientation of the radiopaque tool within a virtual world reference frame. As discussed above with reference to step 204, the two-dimensional image (e.g., a plane representing the two-dimensional image) may be represented within the virtual world reference frame based on the perspective in the virtual world reference frame from which a projection of the three-dimensional model aligns with what is shown in the two-dimensional image. The position and orientation of the radiopaque tool relative to the at least one two-dimensional image may be combined with the position and orientation of the three-dimensional model relative to the two-dimensional image in the virtual world reference frame, resulting in the position and orientation of the radiopaque tool relative to the three-dimensional model in the virtual world reference frame. According to various aspects, the two-dimensional imaging system used to capture the two-dimensional image may be an X-ray imaging system. One or both of the X-ray source and the X-ray detector may be positioned in the virtual world reference frame to determine the position and orientation of the three-dimensional model with respect to the two-dimensional image at which the projection of the three-dimensional model aligns with what is shown in the two-dimensional image while accounting for the X-ray imaging system characteristics.

At step 1106, a position and orientation of the plurality of non-radiopaque objects positioned by the radiopaque tool relative to the three-dimensional model is determined. The position and orientation of the plurality of non-radiopaque objects relative to the three-dimensional model may be determined using the two-dimensional images that were captured when the radiopaque tool was positioning the non-radiopaque objects. As discussed above, a two-dimensional image of the radiopaque tool may be captured when the tip of the radiopaque tool is positioning each non-radiopaque object. The position and orientation of the radiopaque tool relative to the three-dimensional model at the location of each non-radiopaque object is determined based on the two-dimensional images at step 1104. Because each non-radiopaque object was positioned by the radiopaque tool, the position and orientation of the tip of the radiopaque tool relative to the three-dimensional model when the radiopaque tool was positioning a given non-radiopaque object is substantially the same as the position and orientation of the non-radiopaque object relative to the three-dimensional model. Therefore, the position and orientation of each non-radiopaque object relative to the three-dimensional model may be determined by one or more processors of image processing system 106 based on the position and orientation of the tip of the radiopaque tool relative to the three-dimensional model determined from each two-dimensional image.

Determining the position and orientation of the plurality of non-radiopaque objects positioned by the radiopaque tool relative to the three-dimensional model may include determining the position and orientation of the non-radiopaque objects within the virtual world reference frame. The position and orientation of the radiopaque tool within the virtual world reference frame is determined at step 1104. Thus, because the three-dimensional model defines the virtual world reference frame, and the position and orientation of the tip of radiopaque tool relative to the three-dimensional model when the radiopaque tool was positioning a given non-radiopaque object is substantially the same as the position and orientation of the non-radiopaque object relative to the three-dimensional model, the position and orientation of the plurality of non-radiopaque objects within the virtual world can be determined.

At step 1108, an endoscopic image that includes at least some of the plurality of non-radiopaque objects positioned by the radiopaque tool is received. For example, FIG. 7B shows an exemplary endoscopic image 700 in which a plurality of non-radiopaque pins 706 were positioned by a radiopaque tool 708. While the plurality of non-radiopaque objects in FIG. 7B are depicted as pins, it should be understood that any other suitable non-radiopaque objects (e.g., beads, pieces of bone wax, or gel drops) may be used instead of pins. In some examples, the plurality of non-radiopaque objects may include at least two objects in order to provide enough information to determine a location of the objects in the endoscopic image. In some examples, the plurality of non-radiopaque objects may include at least three objects, at least four objects, or at least five objects. The relative arrangement of non-radiopaque pins 706 may not be known in the sense that the non-radiopaque pins 706 may have been placed in relatively arbitrary locations of the bony anatomy such that their positions relative to one another are not initially known to image processing system 106. For example, the non-radiopaque objects 706 may be placed by a surgeon using radiopaque tool 708 after a three-dimensional model of the bony anatomy has already been generated. The image processing system may determine the position of each of the non-radiopaque objects with respect to the three-dimensional model using the techniques described herein.

According to another aspect, the endoscopic image received at step 1108 may include the radiopaque tool in locations associated with the non-radiopaque objects. For example, the endoscopic image may show the tip of the radiopaque tool touching a non-radiopaque object. The endoscopic image including the radiopaque tool may then be used to determine the position and orientation of the non-radiopaque objects in the endoscopic image, as described in greater detail below with reference to step 1110. This may result in a more accurate determination of the position and orientation of the non-radiopaque objects, especially when one or more of the non-radiopaque objects are difficult to visualize in the endoscopic image. In some implementations, endoscopic images of the radiopaque tool touching (or in another known position with respect to) each of the non-radiopaque objects may be obtained at the same time that the two-dimensional images are acquired. This may help to improve the accuracy of the determination of the position and orientation of each non-radiopaque object in the three-dimensional model and the alignment of the three-dimensional model relative to the endoscopic view.

At step 1110, a position and orientation of the plurality of non-radiopaque objects relative to the endoscopic image is determined. Step 1110 may share one or more similarities with step 208 discussed above with reference to FIG. 2. Determining the position and orientation of the plurality of non-radiopaque objects relative to the at least one endoscopic image may include determining a pose of the plurality of non-radiopaque objects with respect to the endoscopic image that results in a projection of the plurality of non-radiopaque objects matching the representation of the plurality of non-radiopaque objects shown in the endoscopic image. As discussed further below, determining the pose of the plurality of non-radiopaque objects with respect to the endoscopic image that results in a projection of the plurality of non-radiopaque objects matching the representation of the plurality of non-radiopaque objects shown in the endoscopic image may involve accounting for the characteristics of the optics of the endoscopic imager used to capture the endoscopic image.

The position and orientation of the plurality of non-radiopaque objects may be determined using image analysis techniques (e.g., an object detection algorithm) to identify the location of the plurality of non-radiopaque objects within the endoscopic image. In some examples, other techniques, such as machine learning (e.g., deep learning), may be used to determine the position and orientation of the non-radiopaque objects relative to the endoscopic image. Characteristics of the optics of the endoscopic imager used to capture the endoscopic image may be used to determine of the position and orientation of the non-radiopaque objects relative to the endoscopic image. Based on the identified location of the plurality of non-radiopaque objects within the endoscopic image and the characteristics of the optics of the endoscopic imager used to capture the endoscopic image, the position and orientation of the plurality of non-radiopaque objects in three-dimensional space that results in a projection of the plurality of non-radiopaque objects matching what is shown in the endoscopic image may be determined.

In some examples, determining the position and orientation of the plurality of non-radiopaque objects relative to the endoscopic image includes determining the position and orientation of the endoscopic imager used to capture the at least one endoscopic image within the virtual world reference frame. For example, a perspective of the endoscopic imager can be determined based on the plurality of non-radiopaque objects shown in the endoscopic image, the known geometry of the plurality of non-radiopaque objects, and one or more characteristics of the optics of the endoscopic imager. This perspective can be combined with the position and orientation of the plurality of non-radiopaque objects within the virtual world reference frame to determine the position and orientation of the endoscopic imager in the virtual world reference frame.

At step 1112, an alignment of the three-dimensional model relative to the endoscopic image is determined based on the position and orientation of the radiopaque tool relative to the three-dimensional model and the position and orientation of the plurality of non-radiopaque objects relative to the endoscopic image. As discussed above with reference to step 1102, the position and orientation of the plurality of non-radiopaque objects relative to the three-dimensional model may be known based the position and orientation of the radiopaque tool relative to the three-dimensional model and the position and the known relationship between the radiopaque tool and the non-radiopaque objects from the two-dimensional images. The position and orientation of the non-radiopaque objects relative to the endoscopic image is also known as a result of step 1110. Thus, the position and orientation of the non-radiopaque objects relative to the three-dimensional model can be combined with the position and orientation of the non-radiopaque objects relative to the endoscopic image to obtain the position and orientation of the three-dimensional model relative to the endoscopic image.

In some examples, not all of the non-radiopaque objects may be visible in the endoscopic image. For example, three non-radiopaque objects may have been placed by the radiopaque tool, but only two of the non-radiopaque objects may be visible from the perspective from which the endoscopic image was taken. Two non-radiopaque objects may not provide enough information to determine an alignment of the three-dimensional model relative to the endoscopic image. Thus, to provide additional context, the radiopaque tool may be positioned so that it is touching the non-radiopaque object that is not visible in the endoscopic image when the endoscopic image is captured.

At step 1114, at least a portion of the three-dimensional model is displayed based on the alignment of the three-dimensional model relative to the endoscopic image. The at least a portion of the three-dimensional model may be displayed in any of the manners discussed above with reference to step 212 of FIG. 2. For example, the at least a portion of the three-dimensional model may be overlaid on or displayed side-by-side with the at least one endoscopic image, or a simulated endoscopic image may be displayed.

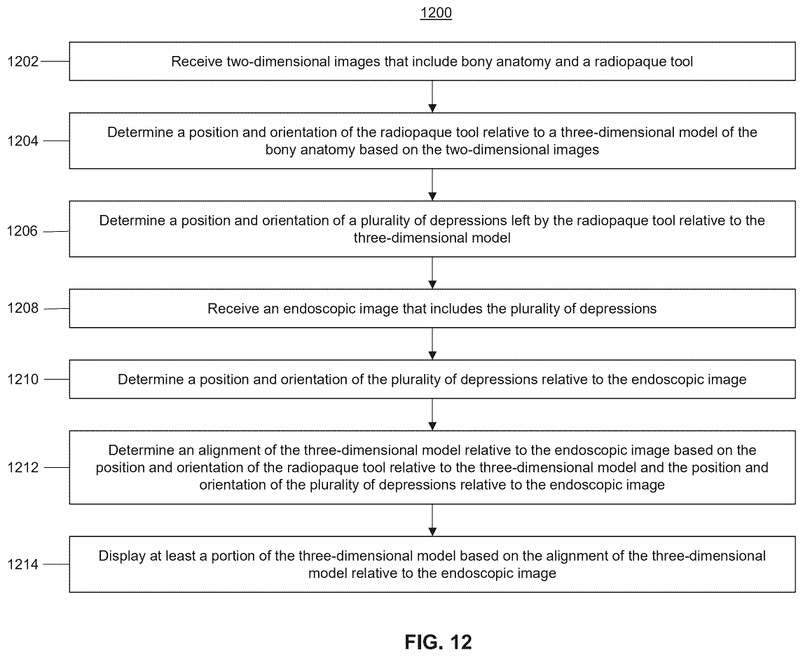

In another example, the at least one radiopaque object of method 200 may be a radiopaque tool used to form depressions in the bony anatomy instead of a radiopaque tool used to position objects relative to the bony anatomy. Attaching objects to bony anatomy can take time and may be difficult, so in some cases, a surgeon may simply create depressions (also referred to herein as “bur pockets”) in the bone. Method 1200 of FIG. 12 illustrates an example implementation of method 200 in which a radiopaque tool and a plurality of depressions are used to determine an alignment of a three-dimensional model relative to at least one endoscopic image. Method 1200 may be performed by one or more processors of image processing system 106 or any other suitable computing system. Aspects of method 1200 are described above with respect to method 200 and are not repeated below for simplicity.

At step 1202, two-dimensional images that include bony anatomy and a radiopaque tool are received. As described above with reference to step 202 of FIG. 2, the two-dimensional images may be fluoroscopic images received, for example, by image processing system 106 from two-dimensional imaging system 110 of FIG. 1A. The two-dimensional images may include the portion of the bone that is being or will be surgically treated, as well as surrounding portions of the bone that enable the surgeon to compare what is shown in the two-dimensional image to what the surgeon sees endoscopically.

The two-dimensional images include a radiopaque tool (e.g., as shown in FIG. 3B discussed above). The radiopaque tool itself may be radiopaque, or the radiopaque tool may include one or more radiopaque markers (e.g., radiopaque beads) affixed to an otherwise non-radiopaque tool. The radiopaque tool may be used to form depressions in the bony anatomy. For example, the radiopaque tool may be a bur, drill, or other bone removal tool configured to form bur pockets in the bony anatomy.

Multiple two-dimensional images are received at step 1202 because multiple depressions may be formed using the radiopaque tool. As discussed above with reference to step 1102 of FIG. 11, the multiple depressions may be formed in arbitrary locations by the surgeon. Thus, image processing system 106 may not possess predetermined knowledge of the position and orientation of the depressions relative to the three-dimensional model that is required to align the three-dimensional model with an endoscopic image that includes the depressions. Accordingly, to determine the position and orientation of the depressions relative to the three-dimensional model, a two-dimensional image of the radiopaque tool may be captured when the tip of the radiopaque tool is forming each depression. The position and orientation of the radiopaque tool relative to the three-dimensional model may be determined based on each two-dimensional image in step 1204 discussed below. Based on the position and orientation of the tip of the tool relative to the three-dimensional model in each two-dimensional image, the position and orientation of each depression relative to the three-dimensional model may be known, since each depression is formed by the radiopaque tool.

At step 1204, a position and orientation of the radiopaque tool relative to a three-dimensional model of the bony anatomy is determined based on the two-dimensional image. Step 1204 may share one or more characteristics with step 204 of FIG. 2. For example, a position and orientation of the radiopaque tool relative to the two-dimensional image may be determined by aligning a projection of a three-dimensional model of the radiopaque tool stored in a memory of image processing system 106 or remote system 108 with the two-dimensional image. A position and orientation of the three-dimensional model of the bony anatomy may also be aligned with the two-dimensional image by aligning a projection of the three-dimensional model into two-dimensional space with the two-dimensional image. The position and orientation of the radiopaque tool relative to the three-dimensional model of the bony anatomy may then be determined based on the position and orientation of the radiopaque object relative to the two-dimensional images and the position and orientation of the three-dimensional model relative to the two-dimensional images. As used herein, determining the position and orientation of the three-dimensional model of the radiopaque tool and the three-dimensional model of bony anatomy relative to the two-dimensional images may include determining a pose of the respective three-dimensional model with respect to the two-dimensional images that results in a projection of the respective three-dimensional model that matches what is shown in the two-dimensional images. Determining the pose of the respective model with respect to the two-dimensional images may include accounting for X-ray system characteristics, as described above.

Determining the position and orientation of the radiopaque tool relative to the three-dimensional model may include determining the position and orientation of the radiopaque tool within the virtual world reference frame. As discussed above with reference to step 204, the two-dimensional image (e.g., a plane representing the two-dimensional image) may be represented within the virtual world reference frame based on the perspective in the virtual world reference frame from which a projection of the three-dimensional model aligns with what is shown in the two-dimensional image. The position and orientation of the radiopaque tool relative to the at least one two-dimensional image may be combined with the position and orientation of the three-dimensional model relative to the two-dimensional image in the virtual world reference frame, resulting in the position and orientation of the radiopaque tool relative to the three-dimensional model in the virtual world reference frame. According to various aspects, the two-dimensional imaging system used to capture the two-dimensional image may be an X-ray imaging system. One or both of the X-ray source and the X-ray detector may be positioned in the virtual world reference frame to determine the position and orientation of the three-dimensional model with respect to the two-dimensional image at which the projection of the three-dimensional model aligns with what is shown in the two-dimensional image while accounting for the X-ray imaging system characteristics.

At step 1206, a position and orientation of a plurality of depressions left by the radiopaque tool relative to the three-dimensional model is determined. The position and orientation of the plurality of depressions relative to the three-dimensional model may be determined using the two-dimensional images that were captured when the radiopaque tool was forming the depressions. As discussed above, a two-dimensional image of the radiopaque tool may be captured when the tip of the radiopaque tool is forming each depression. The position and orientation of the radiopaque tool relative to the three-dimensional model at the location of each depression is determined based on the two-dimensional images at step 1204. Because each depression was formed by the radiopaque tool, the position and orientation of the tip of the radiopaque tool relative to the three-dimensional model when the radiopaque tool was forming a given depression is substantially the same as the position and orientation of the depression relative to the three-dimensional model. Therefore, the position and orientation of each depression relative to the three-dimensional model may be determined by one or more processors of image processing system 106 based on the position and orientation of the tip of the radiopaque tool relative to the three-dimensional model determined from each two-dimensional image.

Determining the position and orientation of the plurality of depressions formed by the radiopaque tool relative to the three-dimensional model may include determining the position and orientation of the depressions within the virtual world reference frame. The position and orientation of the radiopaque tool within the virtual world is determined at step 1204. Thus, because the three-dimensional model defines the virtual world reference frame, and the position and orientation of the tip of radiopaque tool relative to the three-dimensional model when the radiopaque tool was forming a given depression is substantially the same as the position and orientation of the depression relative to the three-dimensional model, the position and orientation of the plurality of depressions within the virtual world can be determined.

At step 1208, an endoscopic image that includes the plurality of depressions left by the radiopaque tool is received. For example, FIG. 7C illustrates an endoscopic image 700 that includes a plurality of depressions 710 (e.g., bur pockets) in the bony anatomy that were created by a radiopaque tool 708. Radiopaque tool 708 may or not may not be pictured in the endoscopic image. The plurality of depressions 710 may include at least two depressions in order to provide enough information to determine a location of the depressions in the endoscopic image. In some examples, the plurality of depressions may include at least three depressions, at least four depressions, or at least five depressions. The relative arrangement of the depressions 710 may not be known in the sense that the depressions 710 may have been formed in relatively arbitrary locations of the bony anatomy such that their positions relative to one another are not initially known to image processing system 106. For example, the depressions 710 may be formed by a surgeon using radiopaque tool 708 after a three-dimensional model of the bony anatomy has already been generated.

According to another aspect, the endoscopic image received at step 1208 may include the radiopaque tool in locations associated with the plurality of depressions. For example, the endoscopic image may show the tip of the radiopaque tool positioned within one of the depressions. The endoscopic image including the radiopaque tool may then be used to determine the position and orientation of the depressions in the endoscopic image, as described in greater detail below with reference to step 1210. This may result in a more accurate determination of the position and orientation of the depressions, especially when one or more of the depressions are difficult to visualize in the endoscopic image. In some implementations, endoscopic images of the radiopaque tool touching (or in another known position with respect to) each of the depressions may be obtained at the same time that the two-dimensional images are acquired. This may help to improve the accuracy of the determination of the position and orientation of each depression in the three-dimensional model and the alignment of the three-dimensional model relative to the endoscopic view.

At step 1210, a position and orientation of the plurality of depressions relative to the endoscopic image is determined. Step 1210 may share one or more similarities with step 208 discussed above with reference to FIG. 2. Determining the position and orientation of the plurality of depressions relative to the at least one endoscopic image may include determining a pose of the plurality of depressions with respect to the endoscopic image that results in a projection of the plurality of depressions matching the representation of the plurality of depressions shown in the endoscopic image. As discussed further below, determining the pose of the plurality of depressions with respect to the endoscopic image that results in a projection of the plurality of depressions matching the representation of the plurality of depressions shown in the endoscopic image may involve accounting for the characteristics of the optics of the endoscopic imager used to capture the endoscopic image.

The position and orientation of the plurality of depressions may be determined using image analysis techniques (e.g., an object detection algorithm) to identify the location of the plurality of depressions within the endoscopic image. In some examples, other techniques, such as machine learning (e.g., deep learning), may be used to determine the position and orientation of the plurality of depressions relative to the endoscopic image. Characteristics of the optics of the endoscopic imager used to capture the endoscopic image may be used to determine the position and orientation of the non-radiopaque objects relative to the endoscopic image. Based on the identified location of the plurality of depressions within the endoscopic image and the characteristics of the optics of the endoscopic imager used to capture the endoscopic image, the position and orientation of the plurality of depressions in three-dimensional space that results in a projection of the plurality of depressions matching what is shown in the endoscopic image may be determined.

In some examples, determining the position and orientation of the plurality of depressions relative to the endoscopic image includes determining the position and orientation of the endoscopic imager used to capture the at least one endoscopic image within the virtual world reference frame. For example, a perspective of the endoscopic imager can be determined based on the depressions shown in the endoscopic image, the known geometry of the depressions, and one or more characteristics of the optics of the endoscopic imager. This perspective can be combined with the position and orientation of the plurality of depressions within the virtual world reference frame to determine the position and orientation of the endoscopic imager in the virtual world reference frame.

At step 1212, an alignment of the three-dimensional model relative to the endoscopic image is determined based on the position and orientation of the radiopaque tool relative to the three-dimensional model and the position and orientation of the plurality of depressions relative to the endoscopic image. As discussed above with reference to step 1202, the position and orientation of the plurality of depressions relative to the three-dimensional model may be known based on the position and orientation of the radiopaque tool relative to the three-dimensional model and a known relationship between the radiopaque tool and the plurality of depressions from the two-dimensional images. The position and orientation of the depressions relative to the endoscopic image is also known as a result of step 1210. Thus, the position and orientation of the depressions relative to the three-dimensional model can be combined with the position and orientation of the depressions relative to the endoscopic image to obtain the position and orientation of the three-dimensional model relative to the endoscopic image.

In some examples, not all of the depressions may be visible in the endoscopic image. For example, three depressions may have been formed by the radiopaque tool, but only two of the depressions may be visible from the perspective from which the endoscopic image was taken. Two depressions may not provide enough information to determine an alignment of the three-dimensional model relative to the endoscopic image. Thus, to provide additional context, the tip of the radiopaque tool may be positioned within the depression that is not visible in the endoscopic image when the endoscopic image is captured.

At step 1214, at least a portion of the three-dimensional model is displayed based on the alignment of the three-dimensional model relative to the endoscopic image. The at least a portion of the three-dimensional model may be displayed in any of the manners discussed above with reference to step 212 of FIG. 2. For example, the at least a portion of the three-dimensional model may be overlaid on or displayed side-by-side with the at least one endoscopic image, or a simulated endoscopic image may be displayed.

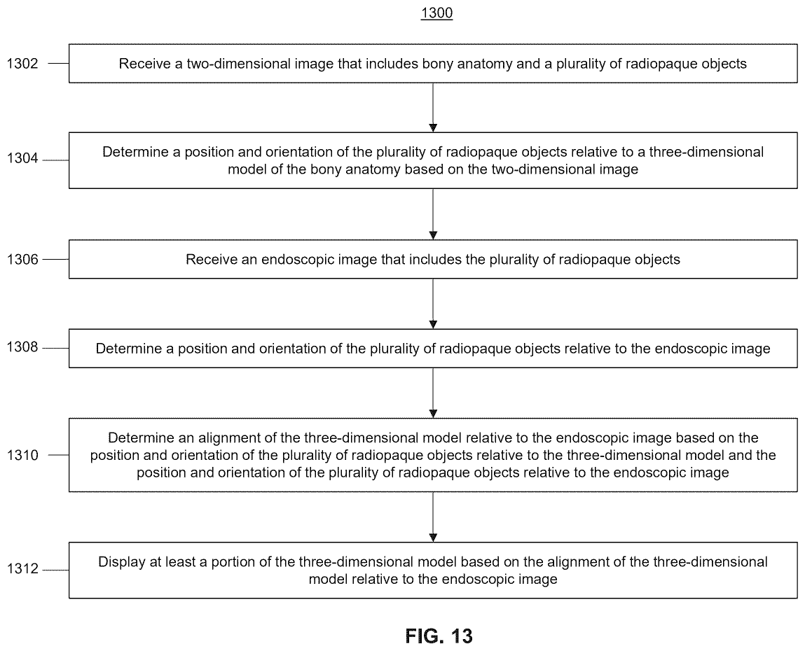

In another example, the at least one radiopaque object of method 200 may be a plurality of radiopaque objects, as discussed above with reference to FIG. 3C, and the at least one feature associated with the at least one radiopaque object may be an optically visible portion of the plurality of radiopaque objects. FIG. 13 illustrates an example implementation of method 200 in which a plurality of radiopaque objects are used to determine an alignment of a three-dimensional model relative to at least one endoscopic image. Method 1300 of FIG. 13 may be performed by one or more processors of image processing system 106 or any other suitable computing system. Aspects of method 1300 are described above with respect to method 200 and are not repeated below for simplicity.

At step 1302, a two-dimensional image that includes bony anatomy and a plurality of radiopaque objects is received. As described above with reference to step 202 of FIG. 2, the two-dimensional image may be a fluoroscopic image received, for example, by image processing system 106 from two-dimensional imaging system 110 of FIG. 1A. The two-dimensional image may include the portion of the bone that is being or will be surgically treated, as well as surrounding portions of the bone that enable the surgeon to compare what is shown in the two-dimensional image to what the surgeon sees endoscopically.

The two-dimensional image includes a plurality of radiopaque objects. Each of the radiopaque objects may be insufficient, alone, to enable the position and orientation determinations described above with respect to the radiopaque object with known geometry. For example, a radiopaque object, such as a pin, may be insufficiently large and/or have an insufficiently unique geometry to determine its position and orientation in the two-dimensional image and/or in an endoscopic image. As such, not enough information may be available in the endoscopic view to determine an accurate alignment of a three-dimensional model to endoscopic images. Accordingly, according to method 1300, multiple radiopaque objects are used, which collectively provide the information needed to determine an accurate alignment of a three-dimensional model to endoscopic images. Any number of radiopaque objects may be used, including at least two radiopaque objects, at least three radiopaque objects, at least four radiopaque objects, or at least five radiopaque objects.

FIG. 3C illustrates an example in which a plurality of radiopaque objects 304 have been inserted into the bony anatomy 302. For example, the radiopaque objects 304 may be placed by a surgeon using an insertion tool. The radiopaque objects 304 may be placed after a three-dimensional model of the bony anatomy has already been generated and, thus, the geometry of the plurality of radiopaque objects may not be known to image processing system 106. The plurality of radiopaque objects 304 may include, but are not limited to, radiopaque pins, radiopaque beads, radiopaque gel drops, or pieces of radiopaque bone wax positioned on the surface of the bony anatomy.

At step 1304, a position and orientation of the plurality of radiopaque objects relative to a three-dimensional model is determined based on the two-dimensional image. As used in this context, the “position and orientation” of the plurality of radiopaque objects refers to the position and orientation of the radiopaque objects as a collective group, rather than the position and orientation of each individual radiopaque object. In some examples, the position and orientation of the plurality of radiopaque objects relative to the three-dimensional model may be determined by determining a plurality of possible locations of the plurality of radiopaque objects relative to the three-dimensional model and selecting a probable location based on an assumed position and orientation of a two-dimensional imaging system (e.g., two-dimensional imaging system 110) used to capture the two-dimensional image relative to the bony anatomy. The plurality of possible locations may include a plurality of possible bone surfaces on which the at least one radiopaque object may be located based on how the two-dimensional imaging system operates, such as opposite surfaces of the bone. Image processing system 106 may estimate which bone surface the plurality of radiopaque objects are on based on a known or assumed position and orientation of the two-dimensional imaging system used to capture the at least one two-dimensional image relative to the bony anatomy. For instance, image processing system 106 may assume that the two-dimensional imaging system is positioned such that any radiopaque objects in a two-dimensional image are on an anterior surface of the bone based on an assumption about the most common positioning of the patient and/or use of the two-dimensional imaging system. Alternatively, image processing system 106 may be provided with a user input indicating the arrangement of the surgical suite (e.g., indicating the position of the patient relative to the two-dimensional imaging system). Based on this known or assumed information, image processing system 106 may determine the location of the plurality of radiopaque objects.

Determining the position and orientation of the plurality of radiopaque objects relative to the three-dimensional model may include determining the position and orientation of the plurality of radiopaque objects within the virtual world reference frame. As discussed above, the position and orientation of the plurality of radiopaque objects relative to the three-dimensional model can be determined by estimating which bone surface the plurality of radiopaque objects are on based on a known or assumed position and orientation of the two-dimensional imaging system used to capture the two-dimensional image relative to the bony anatomy. Because the three-dimensional model defines the virtual world reference frame, the position and orientation of the plurality of radiopaque objects in the virtual world reference frame can be determined. According to various aspects, the two-dimensional imaging system used to capture the two-dimensional image may be an X-ray imaging system. One or both of the X-ray source and the X-ray detector may be positioned in the virtual world reference frame to determine the position and orientation of the three-dimensional model with respect to the two-dimensional image at which the projection of the three-dimensional model aligns with what is shown in the two-dimensional image while accounting for the X-ray imaging system characteristics.

At step 1306, an endoscopic image that includes the plurality of radiopaque objects is received. Step 1306 may be largely similar to step 206 of FIG. 2, except that the “at least one feature associated with the at least one radiopaque object” of step 206 includes optically visible portions of the plurality of radiopaque objects in step 1306. FIG. 7D shows an exemplary endoscopic image 700 illustrating a plurality of small objects 706 inserted into bony anatomy. The plurality of small objects 706 shown in the endoscopic image may include optically visible portions of the plurality of radiopaque objects.

At step 1308, a position and orientation of the plurality of radiopaque objects relative to the endoscopic image is determined. Step 1308 may share one or more similarities with step 208 discussed above with reference to FIG. 2. Determining the position and orientation of the plurality of radiopaque objects relative to the at least one endoscopic image may include determining a pose of the plurality of radiopaque objects with respect to the endoscopic image that results in a projection of the plurality of radiopaque objects matching the representation of the plurality of radiopaque objects shown in the endoscopic image. As discussed further below, determining the pose of the plurality of radiopaque objects with respect to the endoscopic image that results in a projection of the plurality of radiopaque objects matching the representation of the plurality of radiopaque objects shown in the endoscopic image may involve accounting for characteristics of the optics of the endoscopic imager used to capture the endoscopic image.

The position and orientation of the plurality of radiopaque objects may be determined using image analysis techniques to identify the location of the optically visible portions of the plurality of radiopaque objects within the endoscopic image. For example, object detection may be used to identify a known arrangement of the plurality of radiopaque objects in the endoscopic image. The arrangement of the plurality of radiopaque objects may be known from step 1304. In some examples, other techniques, such as machine learning (e.g., deep learning), may be used to determine the position and orientation of the plurality of radiopaque objects relative to the endoscopic image. Characteristics of the optics of the endoscopic imager used to capture the endoscopic image may be used to determine the position and orientation of the plurality of radiopaque objects relative to the endoscopic image. Based on the identified location of the plurality of radiopaque objects within the endoscopic image and the characteristics of the optics of the endoscopic imager used to capture the endoscopic image, the position and orientation of the plurality of radiopaque objects in three-dimensional space that results in a projection of the plurality of radiopaque objects matching what is shown in the endoscopic image may be determined.

In some examples, determining the position and orientation of the plurality of radiopaque objects relative to the endoscopic image includes determining the position and orientation of the endoscopic imager used to capture the endoscopic image within the virtual world reference frame. For example, a perspective of the endoscopic imager can be determined based on the plurality of radiopaque objects shown in the at least one endoscopic image, the known geometry of the plurality of radiopaque objects, and one or more characteristics of the optics of the endoscopic imager. This perspective can be combined with the position and orientation of the plurality of radiopaque objects within the virtual world reference frame to determine the position and orientation of the endoscopic imager in the virtual world reference frame.

At step 1310, an alignment of the three-dimensional model relative to the endoscopic image is determined based on the position and orientation of the plurality of radiopaque objects relative to the three-dimensional model and the position and orientation of the plurality of radiopaque objects relative to the endoscopic image. The position and orientation of the plurality of radiopaque objects is known relative to the three-dimensional model and the endoscopic image from steps 1304 and 1308, respectively. Accordingly, these positions and orientations can be combined to determine an alignment of the three-dimensional model relative to the endoscopic image.

At step 1312, at least a portion of the three-dimensional model is displayed based on the alignment of the three-dimensional model relative to the endoscopic image. The at least a portion of the three-dimensional model may be displayed in any of the manners discussed above with reference to step 212 of FIG. 2. For example, the at least a portion of the three-dimensional model may be overlaid on or displayed side-by-side with the at least one endoscopic image, or a simulated endoscopic image may be displayed.

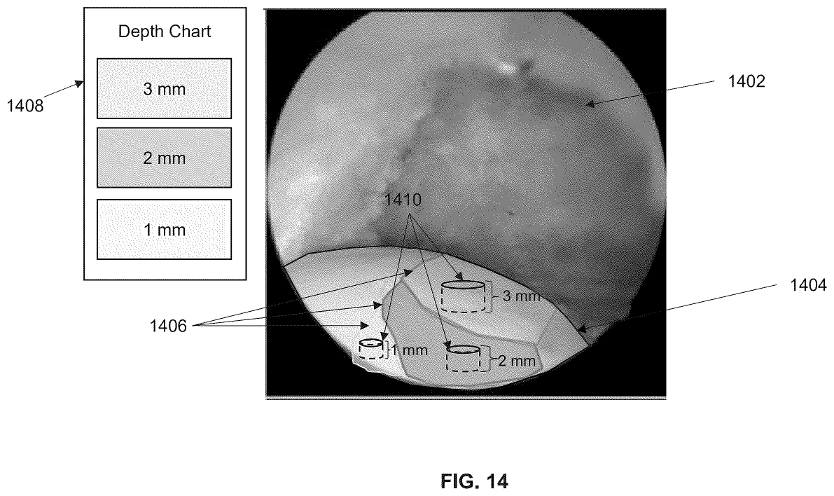

Returning to FIG. 2, in any of the exemplary embodiments discussed above, the displayed three-dimensional model may be used to perform one or more optional steps. For example, at least one depth guide is optionally drilled in the bony anatomy at step 214. A depth guide may indicate to a surgeon the depth of bone to be removed in the vicinity of the drilled depth guide. Based on the depth guide, the surgeon may know how much bone to remove from a given area. In some examples, a depth guide may be based on an indication of planned bone removal (e.g., a heat map). For instance, as shown in FIG. 14, an endoscopic image 1402 may be overlaid with a three-dimensional model 1404. Three-dimensional model 1404 may include a heat map 1406. Heat map 1406 may be color-coded, where different colors indicate differing depths of bone to be removed. Each different color in heat map 1406 may correspond to a different planned bone removal depth in a depth chart 1408. For example, in the example of FIG. 14, the depth of bone to be removed from a first area represented by a first color on the heat map is 3 mm, the depth of bone to be removed from a second area represented by a second color on the heat map is 2 mm, and the depth of bone to be removed from a third area represented by a third color on the heat map is 1 mm. A surgeon may drill a depth guide 1410 in each of the first, second, and third areas based on the depth chart. For example, the surgeon may drill a 3 mm depth guide in the first area represented by the first color, a 2 mm depth guide in the second area represented by the second color, and a 1 mm depth guide in the third area represented by the third color. The surgeon may then remove bone using depth guides 1410 as indicators of roughly how much bone should be removed in different areas of the bony anatomy. For example, the surgeon may resect bone to the depth indicated by each depth guide in the areas surrounding each of the respective depth guides.

Optionally, an alignment of the three-dimensional model relative to endoscopic images is maintained at step 216. As used herein, “maintaining alignment” of the three-dimensional model relative to endoscopic images refers to maintaining the three-dimensional model in a position and orientation relative to endoscopic images such that a projection of the three-dimensional model into two-dimensional space matches the bony anatomy shown in the endoscopic images. An endoscopic imager used to capture the endoscopic images or the bony anatomy captured by the endoscopic imager may move frequently. For example, a patient's leg may be repositioned during surgery, thereby compromising the alignment between the three-dimensional model and the endoscopic images captured by the endoscopic imager. Thus, to accurately convey to the surgeon how the three-dimensional model aligns with what the surgeon is seeing in the endoscopic view, the alignment of the three-dimensional model relative to incoming endoscopic images may be maintained. In some examples, maintaining an alignment of the three-dimensional model relative to endoscopic images includes re-aligning the three-dimensional model with newly received endoscopic images. Re-alignment may be performed in real-time, enabling a surgeon to view an endoscopic video on which the three-dimensional model is overlaid or otherwise aligned with. The alignment process may be performed for each newly received endoscopic image frame or may be performed after a predetermined number of frames (e.g., every other frame, every third frame, every fifth frame, etc.).

An alignment of the three-dimensional model relative to endoscopic images may be maintained by tracking the at least one feature associated with the at least one radiopaque object. Tracking the at least one feature refers to determining how the at least one feature has moved between image frames, and thus how the endoscopic imager has moved relative to the bony anatomy. The at least one feature may be tracked with the endoscopic imager used to capture the endoscopic images. The at least one feature may be any of the options discussed above, such as a fiducial marker with a predetermined pattern (e.g., an ArUco or QR code), a plurality of small objects (e.g., pins, beads, gel drops, or pieces of bone wax), or depressions in the bony anatomy. As long as the at least one feature is visible in the endoscopic view, alignment between the three-dimensional model and endoscopic images may be maintained. For example, during the procedure, the alignment of the three-dimensional model relative to endoscopic images and the bony anatomy may be maintained using the position of one or more fiducial markers, pins, beads, gel drops, pieces of bone wax, or depressions in the bony anatomy that are visible in the endoscopic video. In other words, alignment of the three-dimensional model relative to the endoscopic view and the bony anatomy may be maintained since the position and orientation of the feature(s) to the three-dimensional model are known and the position of the feature(s) can be determined based on the endoscopic video.

In some examples, maintaining alignment between the three-dimensional model and endoscopic images by tracking the at least one feature may not be possible. For example, the endoscopic imager or the bony anatomy may move such that the at least one feature is no longer visible in the endoscopic view. Optionally, if the at least one feature disappears from the endoscopic view, image processing system 106 may generate and output a notification to a surgeon via display 120. The notification may include, for example, a prompt to move the endoscopic imager in a specified direction in order to bring the at least one feature back into view. The prompt may include text-based instructions, arrows indicating a direction in which to move the endoscopic imager, or any other suitable guidance.

To maintain alignment between the three-dimensional model and endoscopic images while the at least one feature is out of view, motion of the endoscopic imager tracked using one or more sensors, such as one or more inertial measurement units (IMUs), may be used to determine a change in position and/or orientation of the endoscopic imager relative to the three-dimensional model. For example, an IMU may provide data about a change in position and/or orientation of the endoscopic imager between a time at which an image used to generate an initial alignment relative to the three-dimensional model was captured and a time at which a new image was captured. The image processing system 106 may analyze this data to determine a change in position and orientation of the endoscopic imager within a virtual world reference frame in order to determine a new alignment of the three-dimensional model relative to the new endoscopic image.

Maintaining alignment between the three-dimensional model and endoscopic images may include aligning the three-dimensional model and the endoscopic imager used to capture the endoscopic images. As discussed above with reference to step 208, the endoscopic imager may be represented in a virtual world reference frame. Posing the endoscopic imager in the virtual world reference frame enables image processing system 106 to update the pose of the imager within the virtual world reference frame using IMU data. The endoscopic imager may be posed in the virtual world reference frame using characteristics of the optics of the endoscopic imager (e.g., focal length). For instance, as discussed above with reference to step 208, a perspective of the endoscopic imager can be determined based on the at least one feature shown in the at least one endoscopic image, the known geometry of the at least one feature, and one or more characteristics of the optics of the endoscopic imager. This perspective can be combined with the position and orientation of the at least one feature within the virtual world reference frame to determine the position and orientation of the endoscopic imager in the virtual world reference frame. Once the endoscopic imager is posed in the virtual world reference frame, motion data generated by the IMU(s) may be used to update the position and orientation of the endoscopic imager in the virtual world reference frame. The updated position and orientation of the endoscopic imager in the virtual world reference frame may be used to determine an updated alignment of the three-dimensional model relative to a newly received image.

For example, an endoscopic image may be captured at time t₀. The endoscopic image captured at to may be used to determine an initial alignment between the three-dimensional model and the endoscopic image at step 210. A new endoscopic image may be captured at time t₁. One or more IMUs associated with the endoscopic imager may record the movement of the endoscopic imager between time to and time t₁. Based on the motion data provided by the IMU(s), image processing system 106 can determine how the endoscopic imager has moved within the virtual world reference frame (and thus relative to the three-dimensional model) between times t₀ and t₁, resulting in an updated alignment between the three-dimensional model and the endoscopic imager. Based on the known characteristics of the optics of the endoscopic imager, the alignment between the three-dimensional model and the endoscopic imager can be transformed into an alignment between the three-dimensional model and the new endoscopic image.

The IMU(s) may be associated with (e.g., attached to or integrated with) the endoscopic imager. For example, as shown in FIG. 1A, one or more IMUs 118 may be associated with endoscopic imager 112. The IMU(s) may include one or more sensors such as accelerometers, gyroscopes, and/or magnetometers. The one or more sensors may detect motion of the endoscopic imager and output motion data, such as linear accelerations in three dimensions, rotational velocities in three dimensions, and/or magnetic field measurements in three dimensions. In some examples, the IMU(s) may be calibrated prior to using motion data from the IMU(s) to determine an alignment between the three-dimensional model and the endoscopic imager, for example at the start of an imaging session. An IMU may be calibrated by placing the endoscopic imager with which the IMU is associated in a known position and orientation and provide an input (e.g., a button press user control device 122 of FIG. 1A) indicating that the IMU can be calibrated. Upon receiving a user input, the readings from the IMU may be saved (e.g., by image processing system 106) as a reference point.

Optionally, calibrating an IMU may include defining an offset associated with a difference in position between the IMU and a reference position on the endoscopic imager. For example, it may be desirable for IMU data to be associated with the tip of the endoscope of the endoscopic imager. An offset between the location of the IMU and the tip of the endoscope may be used to compute a rigid transform between a reference frame centered at the IMU and a reference frame centered at the tip of the endoscope. Different endoscopic imager arrangements (e.g., different camera heads, endoscopes, and/or camera couplers) may have different offsets. Thus, the calibration step may include receiving a user input (e.g., to user control device 122) associated with the endoscopic imager arrangement. The user input may be the offset itself or a selection of a preprogrammed option associated with the imager arrangement. Alternatively, the offset may be calculated using an optical calibration routine (e.g., by generating and analyzing images of a calibration object of known size).

Optionally, motion of the bony anatomy may also be tracked using one or more IMUs, and the corresponding motion data may be used in determining the position and orientation of the three-dimensional model relative to the endoscopic imager. The IMU(s) associated with the bony anatomy may track small movements of the bony anatomy that would otherwise be unaccounted for without the IMU(s)—if the bony anatomy is not tracked by one or more IMUs, the bony anatomy is assumed to be stationary. Thus, measuring small movements of the bony anatomy with the IMU(s) may enable image processing system 106 to determine motion of the endoscopic imager relative to the bony anatomy. Accordingly, using one or more IMU(s) associated with the bony anatomy may result in a more accurate alignment of the three-dimensional model and the endoscopic imager (and thus a more accurate alignment of the three-dimensional model and endoscopic images captured by the endoscopic imager). For example, motion data from the IMU(s) associated with the bony anatomy may be combined with motion data from IMU(s) associated with the endoscopic imager to determine a new alignment of the three-dimensional model relative to the endoscopic imager. The motion data from the IMU(s) associated with the bony anatomy may indicate that the bony anatomy has moved 1 mm in the +x direction, while the motion data from the IMU(s) associated with the endoscopic imager may indicate that the endoscopic imager has moved 2 mm in the −x direction. Using both types of IMU data, image processing system 106 may determine that the endoscopic imager has moved a net distance of 1 mm in the −x direction and may update the alignment of the three-dimensional model relative to the endoscopic imager accordingly. If the bony anatomy did not have associated IMU(s) and was assumed to be stationary, the determination made by image processing system would be incorrect by 1 mm in the −x direction.

Optionally, instead of or in addition to tracking motion of the bony anatomy using one or more IMUs, motion of the bony anatomy may be tracked using an electromagnetic tracking system (e.g., by securing an electromagnetic tracking device to the bony anatomy) and/or an optical tracking system (e.g., by securing a fiducial marker, such as an ArUco, that is visible outside of the body of the subject to the bony anatomy and tracking the fiducial marker with the optical tracking system). Alternatively or additionally, the bony anatomy may be tracked with one or more sensors other than IMUs (e.g., infrared sensors, microwave sensors, ultrasonic sensors, etc.).

The one or more IMUs, tracking devices, or other sensors may be attached to the bony anatomy itself. In aspects involving the hip, the one or more IMUs, tracking devices, or other sensors may be attached to the greater trochanter of the patient. The greater trochanter is easily accessible by a surgeon for implantation, as it is easily located by the surgeon due to its close proximity to the skin. As a result, an IMU, tracking device, or other sensor may be placed percutaneously without the assistance of endoscopic visualization and will not block the movement of surgical instruments during a surgical procedure on the hip. Alternatively, the one or more IMUs, tracking devices, or other sensors may be attached to a piece of surgical equipment to which the bony anatomy is fixed. For example, during a hip surgery, the IMUs, tracking devices, or other sensors may be attached to a distraction frame that holds the leg of the subject in a desired position.

In some examples, the bony anatomy may be tracked using computer vision techniques instead of or in addition to using one or more IMUs, sensors, and/or tracking devices. For example, movement of the bony anatomy may be determined by analyzing endoscopic video captured by the endoscopic imager to track movement of pixels (e.g., a set of pixels corresponding to the bony anatomy) across frames. The endoscopic video may be analyzed using one or more computer vision techniques. For example, the endoscopic video can be analyzed using visual simultaneous localization and mapping (vSLAM) techniques, in which a vSLAM algorithm is used to detect one or more features of the bony anatomy and track the one or more features across frames. Alternatively, the endoscopic video can be analyzed using monocular depth estimation, in which one or more trained machine learning models predict depth values for pixels corresponding to the bony anatomy and track changes in the predicted depth values across frames. Alternatively, the endoscopic video can be analyzed using structured light imaging techniques, in which a predetermined light pattern is projected onto the bony anatomy and distortions in the pattern are analyzed to determine information about the bony anatomy. Each of the above-mentioned computer vision techniques may alternatively or additionally be used to track movement of the bony anatomy based on video captured by one or more cameras external to the subject, rather than by an endoscopic imager.

When the image processing system 106 determines that the bony anatomy has moved based on any of the techniques described above, the image processing system 106 may provide an indication of the movement to a user. The indication may include a warning that the bony anatomy has moved and/or a prompt to the user to re-register the three-dimensional model with respect to the bony anatomy. For example, the prompt may instruct the user to touch a tracked surgical instrument to a location on the bony anatomy that is known relative to the three-dimensional model, as discussed in greater detail below.

Alternatively or in addition, alignment between the three-dimensional model and endoscopic images may be maintained using image analysis techniques. For example, a motion tracking algorithm may be used to process a sequence of image frames and determine relative motion across the frames. The motion tracking algorithm may use optical flow techniques to estimate motion of the endoscopic imager based on the apparent motion of one or more pixels in the sequence of image frames. The motion tracking algorithm may determine frame-to-frame motion between every frame or between a predetermined number of frames (e.g., every other frame, every third frame, etc.). The display of the at least a portion of the three-dimensional model may be updated accordingly. Alternatively, a machine learning model may be used to identify tissue shown in a sequence of endoscopic image frames. For example, a machine learning model may be used to analyze endoscopic images to identify tissue (e.g., by identifying anatomical landmarks). The machine learning model may be trained using a plurality of images of the bone that is being or will be surgically treated and the surrounding tissue. The machine learning model may analyze the endoscopic images to determine the tissue that is shown in the image. Image processing system 106 may match the identified tissue to a corresponding portion of the three-dimensional model and update the displayed three-dimensional model accordingly. In some examples, machine learning models that do not identify tissue may be used to maintain alignment. For example, machine learning models (e.g., deep learning models) may use similarity metrics to determine movement of the endoscopic images relative to the three-dimensional model without identifying anatomical landmarks or specific types of tissue shown in the endoscopic images.

In some examples, the displayed portion of the three-dimensional model may be updated in real-time as bone removal occurs, such that the display reflects the current state of the bone. To update the displayed portion of the three-dimensional model, bone removal may be tracked, and information corresponding to the bone removal may be provided to image processing system 106 to update the three-dimensional model accordingly. Bone removal may be tracked in a variety of ways. For example, bone removal may be tracked by tracking a fiducial marker associated with a bone removal instrument with an endoscopic imager. One or more IMUs may also be used for tracking in case the fiducial marker on the bone removal instrument is not visible (e.g., if the fiducial marker moves out of the endoscopic view). The one or more IMUs may also be used even if the fiducial marker is visible in order to provide additional information about motion of the bone removal instrument, endoscopic imager, and/or bony anatomy. This may improve the accuracy of the updated display of the three-dimensional model.

According to another aspect, bone removal may be tracked by using computer vision techniques to track the bone removal instrument. For example, the bone removal instrument may be tracked by applying a visual simultaneous localization and mapping (vSLAM) algorithm to endoscopic video of the bone removal instrument. The vSLAM algorithm may be configured to detect the bone removal instrument within received endoscopic video and track the bone removal instrument across frames. Optionally, one or more vSLAM algorithms may be used to analyze video captured by a camera external to the subject. For example, one or more vSLAM algorithms may be used to track the movement of the visual fiducial attached to a portion of the bone removal instrument that is external to the body of the subject across a sequence of video frames.

Alternatively, or additionally, bone removal may be tracked using an electromagnetic tracking system to track the bone removal instrument. For example, one or more electromagnetic tracking devices may be attached to the bone removal instrument. Alternatively or additionally, bone removal may be tracked using an optical tracking system including a camera external to the body of the subject. The camera may be an RGB color camera, an infrared camera, or any other type of camera. The camera may track a fiducial marker associated with the bone removal instrument that is external to the subject. For example, the bone removal instrument may include a visual fiducial. The visual fiducial may be located on a portion of the bone removal instrument that is external to the subject and thus outside of the field of view of an endoscopic imager. The visual fiducial may be an ArUco, QR code, or any other visual pattern or marking. The visual fiducial may be tracked by a camera external to the subject to determine the movement of the bone removal instrument.

In some examples, an alert may be provided to a user (e.g., a surgeon or other medical personnel) if the subject has moved while the video is being captured. The alert may prompt the user to re-register the three-dimensional model 402 with respect to the bony anatomy, such as by touching the surgical instrument that includes the visual fiducial to one or more locations on the bony anatomy whose position is known relative to the three-dimensional model 402.

Alternatively or additionally, a bone removal instrument may be tracked by detecting the bone removal instrument in the endoscopic images and determining the location of its bone removing feature. For example, a fiducial marker may be associated with (e.g., affixed to or printed on) the bone removal instrument. The fiducial marker may be an optically visible marker that is visible in the endoscopic view. The fiducial marker may be an ArUco, QR code, marking (e.g., RF marking), or any other predetermined pattern. Motion of the bone removal instrument may be monitored by keeping the fiducial in the endoscopic view. When the endoscopic images show that a bone removal portion of the bone removal instrument is removing bone, image processing system 106 may update the three-dimensional model accordingly based on the endoscopic images. In some examples, one or more machine learning (e.g., deep learning) models may be used to identify the location of the bone removal portion of the bone removal instrument in the endoscopic images and determine that the bone removal portion is removing bone.

Alternatively or in addition, the bone removal instrument may be tracked using one or more IMUs. The one or more IMUs may be affixed to or integrated into the bone removal instrument. When a portion of the bone removal instrument used to resect bone is determined to be in a location that is known to correspond to bony anatomy, the three-dimensional model may be updated by removing the portion of the three-dimensional model that corresponds to the overlap between the portion of the bone removal instrument and the three-dimensional model. Using one or more IMUs may be particularly helpful if the bone removal instrument moves out of the endoscopic view. In that case, a fiducial associated with the bone removal instrument can no longer be tracked using the endoscopic imager, and the one or more IMUs may be used to track the bone removal instrument. In some examples, the one or more IMUs associated with the bone removal instrument may be used to supplement tracking of a fiducial associated with the bone removal instrument to improve the accuracy of the updated three-dimensional model.

The information derived from any of the tracking techniques discussed above may be used by image processing system 106 to update the three-dimensional model to reflect bone removal. When a portion of the tracked bone removal instrument used to resect bone is determined to be in a location that is known to correspond to bony anatomy, the three-dimensional model may be updated to reflect removal of the portion of the three-dimensional model that corresponds to the overlap between the portion of the bone removal instrument and the three-dimensional model.

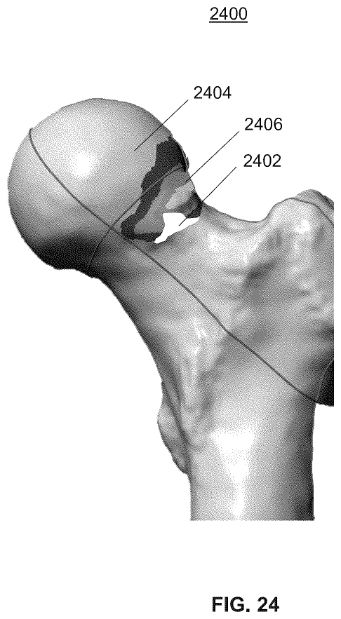

According to an aspect, the three-dimensional model may be updated to include an indication of portions of bone that have been resected. For example, the three-dimensional model may be updated to show the portion(s) of bone that have been removed with a different color and/or level of transparency than the rest of the bone. FIG. 24 illustrates an example of a three-dimensional model 2400 in which a portion of bone that has been resected 2402 is rendered using a lighter color than non-resected portions of bone 2404. As shown in FIG. 24, the heat map 2406 may optionally be removed in the area corresponding to the portion of bone that has been resected 2402. In some examples, the portion of bone that has been resected 2402 may be rendered using a different level of transparency than the non-resected portions of bone 2404 instead of or in addition to using a different coloration. Rendering resected areas using a different color and/or level of transparency may indicate to a surgeon where bone has been resected. Optionally, in aspects where different coloration is used to indicate the portion of bone that has been resected 2402, the color may indicate how recently the bone was resected. For example, portion 2402 may be shown in a first color if the bone was resected in the last minute and may be shown in a second color (e.g., a lighter color) if it was resected over one minute ago. The color may change at a fixed or variable rate. In implementations where the color changes at a fixed rate, the color may fade or change continuously with time or may change color or brightness according to a step function. Similarly, in aspects where different levels of transparency are used to indicate the portion of bone that has been resected 2402, the level of transparency may indicate how recently the bone was resected. For example, portion 2402 may be shown using a first level of transparency if the bone was resected in the last minute and may be shown using a second level of transparency (e.g., either more or less transparent) if it was resected over one minute ago. The level of transparency may change with time at a fixed or variable rate. It should be understood that the level of transparency may change over times other than one minute, according to various aspects.

In some examples, other system components may be tracked using IMUs, sensors, computer vision techniques, or any of the other tracking techniques described herein instead of or in addition to the bone removal instrument. For example, one or more IMUs may be associated with the endoscopic imager. Tracking motion of the endoscopic imager using one or more IMUs enables image processing system 106 to determine the motion of the bone removal instrument relative to the endoscopic imager. This may result in a more accurate determination of bone removal and thus a more accurate updated three-dimensional model. In some examples, one or more IMUs may also be associated with the bony anatomy itself. Tracking the bony anatomy with one or more IMUs enables image processing system 106 to determine whether the patient has moved, which may also result in a more accurate updated three-dimensional model. Tracking the endoscopic imager and/or the bony anatomy with one or more IMUs may also help to maintain alignment of the three-dimensional model to the endoscopic view while bone is being removed.

Alignment of the three-dimensional model to the endoscopic view may also be maintained during bone removal by placing the tip of the bone removal instrument at a location that is known relative to the three-dimensional model and capturing an endoscopic image. For example, the tip of the bone removal instrument may be placed at a location on the bony anatomy that is known relative to the three-dimensional model, such as in a bur pocket or touching an object placed on or in the bony anatomy (e.g., a pin, bead, piece of bone wax, a gel drop, etc.). Alternatively or additionally, the tip of the bone removal instrument may be placed at a location external to the patient that is known relative to the three-dimensional model (e.g., a predetermined location on a surgical table, an exterior portion of the patient's anatomy, or any other suitable external position within the surgical suite). The position and orientation of the bur pocket, object, or other location relative to the three-dimensional model may be known (e.g., from performing one of methods 200, 1100, 1200, or 1300). The position and orientation of the tip of the bone removal instrument in the endoscopic image may be determined, for example by using image processing techniques. For instance, image processing techniques may include using one or more object detection algorithms and/or machine learning (e.g., deep learning) techniques. Because the position and orientation of the tip of the bone removal instrument is known relative to the endoscopic image, and the tip of the bone removal instrument is touching the bur pocket or object, the position and orientation of the bur pocket or object in the endoscopic image is known. As noted above, the position and orientation of the bur pocket or object relative to the three-dimensional model is already known. Thus, the position and orientation of the three-dimensional model relative to the endoscopic image can be determined. In some examples, a surgeon may repeat this process throughout a surgical procedure in order to maintain alignment of the three-dimensional model relative to the endoscopic view. The surgeon may repeat this process periodically (e.g., at regular time intervals throughout the surgical procedure) and/or when the alignment is disrupted (e.g., if the surgeon moves the bony anatomy during the surgical procedure).

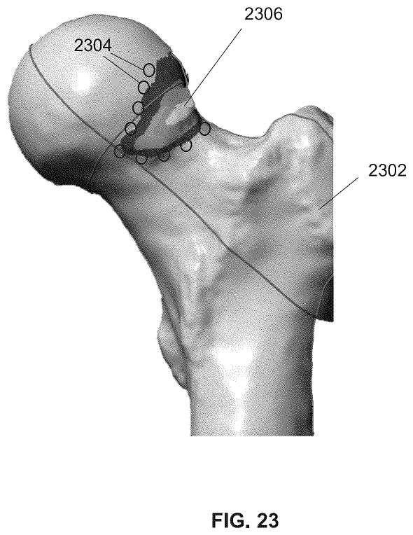

According to an aspect, the location(s) known relative to the three-dimensional model may be bur pockets or small divots formed in the bony anatomy along the perimeter of a planned bone removal area. For example, a surgeon may form 2, 3, 4, 5, 10, 15, or more bur pockets along the perimeter of a planned bone removal area. The bur pockets may correspond to the outer perimeter of an indication of planned bone removal (e.g., a heat map) displayed with respect to the three-dimensional model. FIG. 23 illustrates an example of a three-dimensional model of bony anatomy 2302 having a plurality of bur pockets 2304 distributed along the perimeter of a planned bone removal area 2306, which is indicated in FIG. 23 using a heat map. In some implementations, the position of the bony anatomy in the virtual world reference frame may be known, and the bur to be used to form the bur pockets 2304 may be tracked relative to the virtual world reference frame prior to forming the bur pockets, such that the surgeon can visualize where the bur pockets 2304 are being formed in the virtual world reference frame. The bur pockets 2304 may then be used for re-registering the three-dimensional model with respect to the bony anatomy (and thus with respect to the endoscopic view) if their alignment becomes disrupted. For example, if the patient's leg is moved during a procedure on a hip, a tracked surgical instrument may be touched to one or more of the bur pockets 2304. Since the position and orientation of the surgical instrument relative to endoscopic video is known, and the tip of the surgical instrument is touching the bur pocket, the position and orientation of the bur pocket in the endoscopic image is known. And since the position and orientation of the bur pocket relative to the three-dimensional model is known, the position and orientation of the three-dimensional model relative to the endoscopic view can be re-established. According to other aspects, different types of marks that are visible in the endoscopic image other than bur pockets may be placed along the perimeter of a planned bone removal area. For example, one or more RF markings or etched lines may be used to mark the perimeter of a planned bone removal area.

Forming numerous bur pockets 2304 (as opposed to forming one or very few bur pockets) can be advantageous because having more bur pockets 2304 available provides more options for re-registering the three-dimensional model with respect to the bony anatomy and the endoscopic view. This is especially important because the bur pockets 2304 may be removed or obscured over the course of a bone removal procedure. In addition to providing options for re-registration, bur pockets 2304 may also serve as visual guidance for bone removal, since they are placed along the perimeter of the area where bone is to be removed. Moreover, bur pockets 2304 may be used to maintain alignment between the three-dimensional model, the endoscopic view, and the bony anatomy using information from the endoscopic video. For example, during the procedure, the alignment of the three-dimensional model relative to the endoscopic view and the bony anatomy may be maintained using the positions of the bur pockets 2304 that are visible in the endoscopic video (e.g., alignment of the three-dimensional model relative to the endoscopic view and the bony anatomy may be maintained since the position and orientation of the bur pockets relative to the three-dimensional model are known and the positions of the bur pockets can be determined based on the endoscopic video). In other words, it may be possible to maintain alignment between the three-dimensional model and the endoscopic view and the bony anatomy without using touching a surgical instrument to the bur pockets.

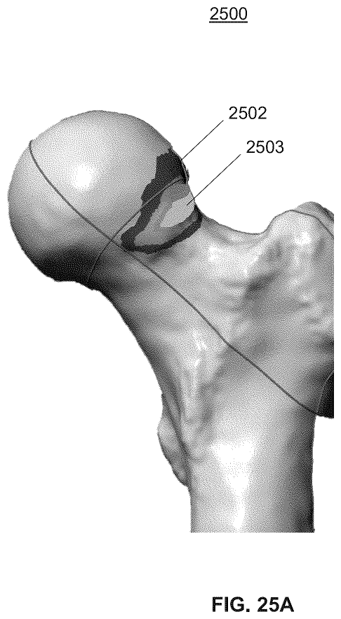

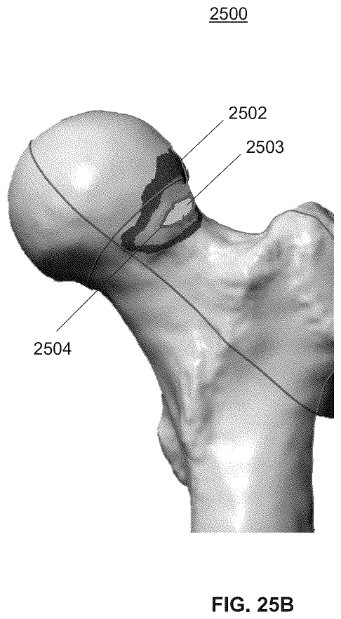

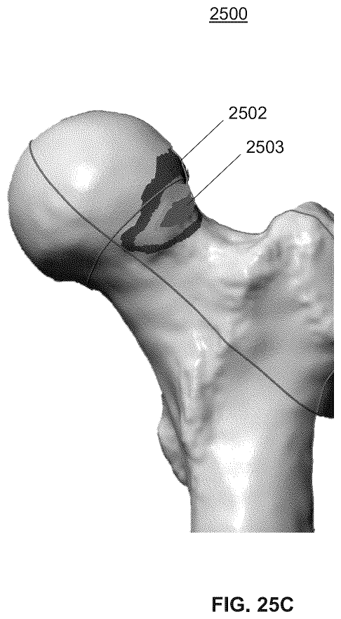

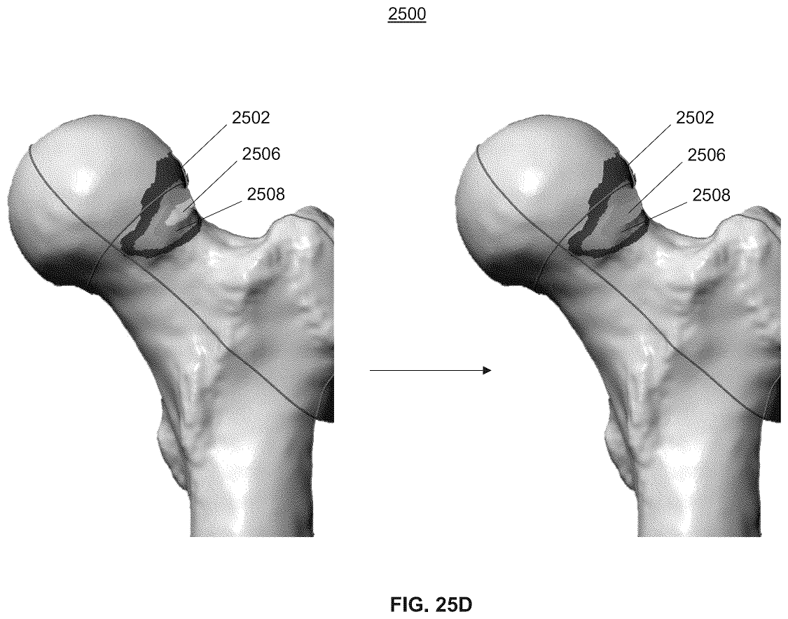

As discussed above, the three-dimensional model may be updated to reflect bone removal. For example, bone corresponding to an area presumed to have been resected may be removed from the model. According to other aspects, bone removal may be indicated by without completely removing the bone from the model. For example, a heat map displayed on the three-dimensional model may be updated to reflect the removal of bone. For example, a region of the heat map corresponding to bone that has recently been resected may be removed, highlighted, colorized, or the like. In some examples, a region of the heat map corresponding to bone that has been resected may be removed. For example, FIG. 25A illustrates a visualization in which a portion 2503 of heat map 2502 corresponding to bone that has been resected has been removed, such that the three-dimensional model of the bone is visible in the region of portion 2503. Removal of portion 2503 may indicate that bone in the area represented by portion 2503 has been resected to the target depth. In some examples, the portion 2503 of heat map 2502 that has been removed may be indicated using an outline 2504, as shown in FIG. 25B, which may help a surgeon better visualize which areas of bone have been resected to their target depth. According to another aspect, a region of the heat map corresponding to bone that has been resected to a target depth may be indicated using a predetermined color. For example, as shown in FIG. 25C, the portion 2503 of heat map 2502 corresponding to bone that has been resected to its target depth may be shown using a color that is not used in the heat map to represent bone to be removed. In FIG. 25C, portion 2503 is indicated using purple, since purple is not used to represent anything else in the heat map.

Additionally, or alternatively, a region of the heat map corresponding to bone that has recently been resected may be highlighted using a predetermined color to indicate that the bone in that location was recently removed. In some examples, different color indicators may be used in the heat map to indicate the recency of bone removal (e.g., a portion of the heat map corresponding to bone that was removed one minute ago may be brighter or darker than a portion of the heat map corresponding to bone that was removed five minutes ago). The color indicators may change at a fixed or variable rate. In implementations where the color indicators change at a fixed rate, the color of a recently removed region of the heat map may fade or change continuously with time or may change color or brightness according to a step function. Additionally, or alternatively, the color of a region of the heat map corresponding to a region of removed bone may be updated to reflect how much more bone in that region should be removed. For example, with reference to FIG. 25D, a first portion 2506 of a heat map 2502 may be colored yellow to indicate that 3 mm of bone remains to be resected in that area, and a second portion 2508 of heat map 2502 may be colored green to indicate that 2 mm of bone remains to be resected in that area. If 1 mm of bone is resected from first portion 2506, the color of first portion 2506 may change from yellow to green to reflect that 2 mm of bone now remains to be resected in first portion 2506. The color of first portion 2506 may change in real-time as bone is resected. Optionally, the color may fade gradually as bone is resected, which may indicate to a surgeon where resection has recently occurred.

Optionally, one or more interpolation and/or smoothing operations may be performed, such that resected areas of bone in the three-dimensional model are shaped like valleys with relatively smooth edges rather than holes with jagged or abrupt edges. The updated three-dimensional model may be shown to the surgeon while the bone removal instrument is removing bone so that the surgeon can see where and how much bone has been removed and still needs to be removed in real-time.

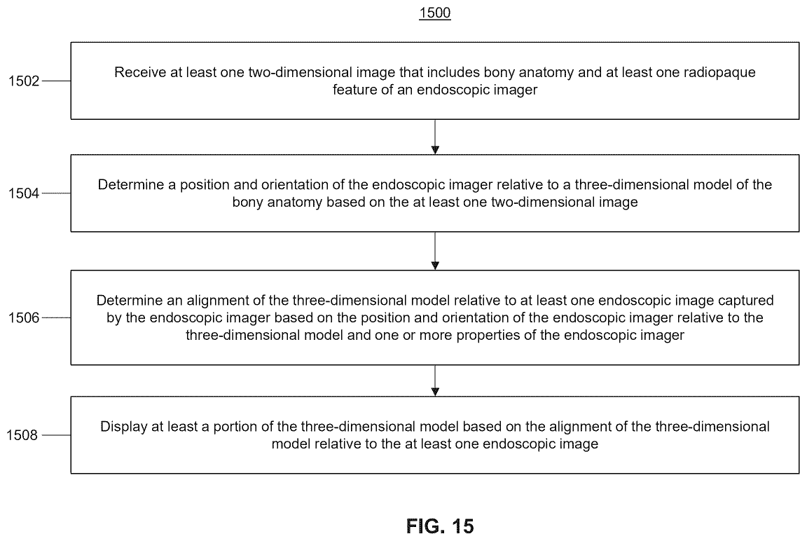

In some examples, rather than using objects or depressions associated with bony anatomy to determine an alignment of a three-dimensional model to an endoscopic image, the endoscopic imager itself may be used. FIG. 15 illustrates an exemplary method 1500 for guiding bone removal in which radiopaque features of an endoscopic imager are used to align a three-dimensional model to an endoscopic image. Method 1500 may be performed by one or more processors of image processing system 106 or any other suitable computing system. By performing method 1500, image processing system 106 may generate a visualization for display by display 120 that includes at least a portion of a three-dimensional model of bony anatomy displayed in a position and orientation that aligns with the bony anatomy in an endoscopic image. Method 1500 may be advantageous because it does not require a surgeon to insert any objects into the surgical site or form any depressions in the bony anatomy in order to align a three-dimensional model to an endoscopic image. Rather, the endoscopic imager may include radiopaque features that enable alignment of the endoscopic imager relative to the three-dimensional model, and calibrated optics of the endoscopic imager may be used to transform the alignment of the endoscopic imager relative to the three-dimensional model to alignment of an endoscopic image relative to the three-dimensional model.

At step 1502, at least one two-dimensional image that includes bony anatomy and at least one radiopaque feature of an endoscopic imager is received. As described above with reference to step 202 of FIG. 2, the at least one two-dimensional image may be a fluoroscopic image received, for example, by image processing system 106 from two-dimensional imaging system 110 of FIG. 1A. The two-dimensional image(s) may include the portion of the bone that is being or will be surgically treated, as well as surrounding portions of the bone that enable the surgeon to compare what is shown in the two-dimensional image(s) to what the surgeon sees endoscopically.

The at least one two-dimensional image includes at least one radiopaque feature of the endoscopic imager. The at least one radiopaque feature may be one or more radiopaque objects or markings associated with the endoscopic imager. For example, the at least one radiopaque feature may include radiopaque beads or pins affixed to the endoscopic imager or radiopaque gel drops or paint placed on the endoscopic imager. Alternatively, the at least one radiopaque feature of the endoscopic imager may be a feature of the endoscopic imager itself. For instance, at least a portion of the endoscopic imager may be made from a radiopaque material.

At step 1504, a position and orientation of the endoscopic imager relative to a three-dimensional model of the bony anatomy is determined based on the at least one two-dimensional image. The at least one two-dimensional image includes at least a portion of the endoscopic imager. The geometry of the endoscopic imager may be known. For example, a three-dimensional model of the endoscopic imager may be stored in a memory of image processing system 106 or in remote system 108. A position and orientation of the endoscopic imager relative to the at least one two-dimensional image may thus be determined by determining a projection of the model of the endoscopic imager into two-dimensional space that aligns with the at least one two-dimensional image. In addition, a position and orientation of the three-dimensional model of the bony anatomy relative to the at least one two-dimensional image may be determined. Both the position and orientation of the endoscopic imager relative to the at least one two-dimensional image and the position and orientation of the three-dimensional model relative to the at least one two-dimensional image may be determined using method 500 or any other suitable alignment method. The endoscopic imager and the three-dimensional model are thus both aligned with the at least one two-dimensional image. Accordingly, the endoscopic imager and the three-dimensional model may be aligned with each other.

At step 1506, an alignment of the three-dimensional model relative to at least one endoscopic image captured by the endoscopic imager is determined based on the position and orientation of the endoscopic imager relative to the three-dimensional model and one or more properties of the endoscopic imager. The one or more properties of the endoscopic imager may include calibrated optics of the endoscopic imager. The position and orientation of the endoscopic imager relative to objects and/or anatomical structures in the endoscopic images captured by the endoscopic imager may be known as a result of calibrating the endoscopic imager. During calibration, the endoscopic imager may capture an endoscopic image of a calibration object with a known pattern and/or geometry. The known characteristics of the calibration object may be compared to the characteristics of the calibration object in the endoscopic image to determine characteristics of the optics of the scope (e.g., the focal length and/or rotation of the endoscope of the endoscopic imager). For example, in some implementations, the endoscopic imager may be positioned within a range of distances from a field of calibration markers that corresponds to the typical working range of the endoscopic imager during surgery. The endoscopic imager may be positioned using a calibration fixture, which may include a plurality of ports for positioning the endoscope in different positions relative to the field of calibration markers. The endoscopic imager may be used to capture a plurality of images of the field of calibration markers from the different positions. The field of calibration markers may include a plurality of visual markers (e.g., ArUco markers, bar codes, QR codes, etc.) arranged in a known pattern. Attributes of the calibration markers in the images may be compared to known attributes of the calibration markers. Calibration factors corresponding to the optics of the scope (e.g., an initial camera matrix and distortion coefficients) may be generated based on the comparison of the attributes.

The position and orientation of endoscopic images captured by the endoscopic imager may then be determined based on the calibrated optics. Because the position and orientation of the endoscopic imager relative to the three-dimensional model is known as a result of step 1504, and the relationship between the endoscopic imager and objects and/or anatomical structures in the endoscopic images is known based on the calibration optics, the alignment of the three-dimensional model relative to at least one endoscopic image captured by the endoscopic imager may be determined.

At step 1508, at least a portion of the three-dimensional model is displayed based on the alignment of the three-dimensional model relative to the at least one endoscopic image. The at least a portion of the three-dimensional model may be displayed in any of the manners discussed above with reference to step 212 of FIG. 2. For example, the at least a portion of the three-dimensional model may be overlaid on or displayed side-by-side with the at least one endoscopic image, or a simulated endoscopic image may be displayed.

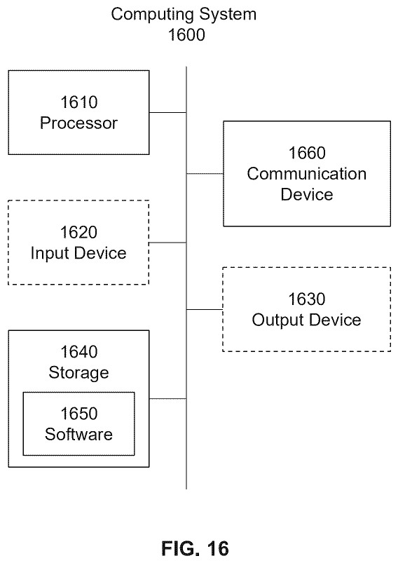

FIG. 16 illustrates an example of a computing system 1600 that may be used in any one of the systems described herein, such as in image processing system 106 of FIG. 1A. System 1600 can be a computer connected to a network. System 1600 can be a client computer or a server. As shown in FIG. 16, system 1600 can be any suitable type of microprocessor-based system, such as a personal computer, workstation, server, or handheld computing device (portable electronic device) such as a phone or tablet. The system can include, for example, one or more of a processor 1610, storage 1640, and communication device 1660.

Optionally, system 1600 may include an input device 1620 and/or an output device 1630. Input device 1620 can be any suitable device that provides input, such as a touch screen, keyboard or keypad, mouse, gesture recognition component of a virtual/augmented reality system, or voice-recognition device. Output device 1630 can be or include any suitable device that provides output, such as a display, touch screen, haptics device, virtual/augmented reality display, or speaker. Input device 1620 and output device 1630 can be integrated into system 1600. Alternatively, external input devices and/or output devices that are not a part of system 1600 may be used in conjunction with system 1600. For example, in FIG. 1A, system 1600 may be used in image processing system 106, and a user control device 122 and a display 120 may be connected to image processing system 106.

Storage 1640 can be any suitable device that provides storage, such as an electrical, magnetic, or optical memory, including a RAM, cache, hard drive, removable storage disk, or other non-transitory computer readable medium. Communication device 1660 can include any suitable device capable of transmitting and receiving signals over a network, such as a network interface chip or device. The components of the computer can be connected in any suitable manner, such as via a physical bus or wirelessly.

Software 1650, which can be stored in storage 1640 and executed by processor 1610, can include, for example, the programming that embodies the functionality of the present disclosure (e.g., as embodied in the devices as described above). For example, software 1650 can include one or more programs for performing one or more of the steps of method 200, method 1100, method 1200, method 1300, and/or method 1500.

Software 1650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1640, that can contain or store programming for use by or in connection with an instruction execution system, apparatus, or device.

Software 1650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

System 1600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 1600 can implement any operating system suitable for operating on the network. Software 1650 can be written in any suitable programming language, such as C, C++, Java, or Python. In various aspects, application software embodying the functionality of the present disclosure can be deployed in different configurations, such as in a client/server arrangement or through a Web browser as a Web-based application or Web service, for example.

The foregoing description, for the purpose of explanation, has been described with reference to specific aspects. However, the illustrative discussions above are not intended to be exhaustive or to limit the invention to the precise forms disclosed. Many modifications and variations are possible in view of the above teachings. The aspects were chosen and described in order to best explain the principles of the techniques and their practical applications. Others skilled in the art are thereby enabled to best utilize the techniques and various aspects with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of any patents and publications referred to in this application are hereby incorporated herein by reference.

1. A method for guiding bone removal comprising:
receiving at least one two-dimensional image comprising bony anatomy and at least one radiopaque object;
determining a position and orientation of the at least one radiopaque object relative to a three-dimensional model of the bony anatomy based on the at least one two-dimensional image;
receiving at least one endoscopic image comprising at least one feature associated with the at least one radiopaque object;
determining a position and orientation of the at least one feature associated with the at least one radiopaque object relative to the at least one endoscopic image;
determining an alignment of the three-dimensional model relative to the at least one endoscopic image based on the position and orientation of the at least one radiopaque object relative to the three-dimensional model and the position and orientation of the at least one feature associated with the at least one radiopaque object relative to the at least one endoscopic image; and
displaying at least a portion of the three-dimensional model based on the alignment of the three-dimensional model relative to the at least one endoscopic image.

2. The method of claim 1, wherein determining a position and orientation of the at least one radiopaque object relative to the three-dimensional model of the bony anatomy based on the at least one two-dimensional image comprises:
determining a position and orientation of the three-dimensional model relative to the at least one two-dimensional image; and
determining a position and orientation of the at least one radiopaque object relative to the at least one two-dimensional image.

3. The method of claim 1, wherein determining a position and orientation of the at least one radiopaque object relative to a three-dimensional model of the bony anatomy based on the at least one two-dimensional image comprises:
determining a plurality of possible locations of the at least one radiopaque object relative to the three-dimensional model; and
selecting a location of the at least one radiopaque object relative to the three-dimensional model based on a position and orientation of a two-dimensional imager used to capture the at least one two-dimensional image relative to the bony anatomy.

4. The method of claim 1, wherein displaying the at least a portion of the three-dimensional model comprises displaying an overlay of the at least a portion of the three-dimensional model on the at least one endoscopic image.

5. The method of claim 4, wherein the overlay is at least partially transparent.

6. The method of claim 1, wherein displaying the at least a portion of the three-dimensional model comprises displaying the at least a portion of the three-dimensional model separately from the at least one endoscopic image.

7. The method of claim 1, wherein displaying the at least a portion of the three-dimensional model comprises displaying a simulated endoscopic image that comprises the at least a portion of the three-dimensional model.

8. The method of claim 1, wherein the at least a portion of the three-dimensional model that is displayed comprises an indication of planned bone removal.

9. The method of claim 8, wherein the indication of planned bone removal comprises a heat map.

10. The method of claim 1, wherein the at least one feature associated with the at least one radiopaque object comprises a fiducial marker, a shape of at least a portion of the at least one radiopaque object, one or more markings in the bony anatomy made by a tool, bone wax or gel drops deposited by the tool, one or more depressions formed in the bony anatomy by the tool, or one or more pins inserted into the bony anatomy by the tool.

11. The method of claim 1, wherein the at least one radiopaque object comprises at least one radiopaque object anchored to the bony anatomy, at least one radiopaque material deposited on the bone, a tool for positioning the at least one feature associated with the at least one radiopaque object with respect to the bony anatomy, or a radiopaque fiducial attached to the tool for positioning the at least one feature associated with the at least one radiopaque object with respect to the bony anatomy.

12. The method of claim 1, wherein the at least one radiopaque object comprises a plurality of radiopaque objects, and the at least one feature associated with the at least one radiopaque object comprises an optically visible portion of at least one of the plurality of radiopaque objects.

13. The method of claim 1, wherein the at least one feature associated with the at least one radiopaque object is not radiopaque.

14. The method of claim 1, further comprising:
receiving a new endoscopic image comprising the at least one feature associated with the at least one radiopaque object;
determining a position and orientation of the at least one feature associated with the at least one radiopaque object relative to the new endoscopic image; and
determining an alignment of the three-dimensional model relative to the new endoscopic image based on the position and orientation of the at least one radiopaque object relative to the three-dimensional model and the position and orientation of the at least one feature associated with the at least one radiopaque object relative to the new endoscopic image.

15. The method of claim 14, further comprising updating the display of the at least a portion of the three-dimensional model based on the alignment of the three-dimensional model relative to the new endoscopic image.

16. The method of claim 1, comprising monitoring motion of an endoscopic imager based on a plurality of endoscopic images or at least one inertial measurement unit and updating the display of the at least a portion of the three-dimensional model based on the monitored motion of the endoscopic imager.

17. The method of claim 1, comprising monitoring motion of the bony anatomy using at least one inertial measurement unit and updating the display of the at least a portion of the three-dimensional model based on the monitored motion of the bony anatomy.

18. The method of claim 1, further comprising tracking a bone removal instrument while the bone removal instrument removes bone and updating the at least a portion of the three-dimensional model to reflect bone removal based on the tracking of the bone removal instrument.

19. The method of claim 18, wherein tracking the bone removal instrument comprises analyzing endoscopic images to determine a position and orientation of the bone removal instrument or tracking motion of the bone removal instrument using at least one inertial measurement unit.

20. The method of claim 19, wherein analyzing the endoscopic images to determine the position and orientation of the bone removal instrument comprises analyzing a fiducial marker associated with the bone removal instrument.

21. The method of claim 18, comprising, while tracking the bone removal instrument, maintaining alignment of the three-dimensional model relative to endoscopic images based on at least one fiducial attached to the bone.

22. The method of claim 18, comprising, while tracking the bone removal instrument, maintaining alignment of the three-dimensional model relative to endoscopic images by:
analyzing the endoscopic images to identify at least a portion of the bony anatomy in the endoscopic images; and
matching the at least a portion of the bony anatomy in the endoscopic images to a corresponding portion of the three-dimensional model.

23. The method of claim 18, comprising, while tracking the bone removal instrument, maintaining alignment of the three-dimensional model relative to endoscopic images by:
receiving endoscopic images comprising the bone removal instrument positioned at a location of the at least one feature associated with the at least one radiopaque object;
determining a position and orientation of the bone removal instrument relative to the endoscopic images; and
determining a position and orientation of the three-dimensional model relative to the endoscopic images based on the position and orientation of the bone removal instrument relative to the endoscopic images and a position and orientation of the at least one feature associated with the at least one radiopaque object relative to the three-dimensional model.

24. The method of claim 1, comprising:
while displaying the at least a portion of three-dimensional model, drilling at least one depth guide in the bony anatomy; and
removing bone based on the at least one depth guide.

25. A method for guiding bone removal comprising:
receiving at least one two-dimensional image comprising bony anatomy and at least one radiopaque feature of an endoscopic imager;
determining a position and orientation of the endoscopic imager relative to a three-dimensional model of the bony anatomy based on the at least one two-dimensional image;
determining an alignment of the three-dimensional model relative to at least one endoscopic image captured by the endoscopic imager based on the position and orientation of the endoscopic imager relative to the three-dimensional model and one or more properties of the endoscopic imager; and
displaying at least a portion of the three-dimensional model based on the alignment of the three-dimensional model relative to the at least one endoscopic image.

26. A system for guiding bone removal comprising one or more processors and memory storing one or more programs for execution by the one or more processors, the one or more programs including instructions that, when executed by the one or more processors, cause the system to:
receive at least one two-dimensional image comprising bony anatomy and at least one radiopaque object;
determine a position and orientation of the at least one radiopaque object relative to a three-dimensional model of the bony anatomy based on the at least one two-dimensional image;
receive at least one endoscopic image comprising at least one feature associated with the at least one radiopaque object;
determine a position and orientation of the at least one feature associated with the at least one radiopaque object relative to the at least one endoscopic image;
determine an alignment of the three-dimensional model relative to the at least one endoscopic image based on the position and orientation of the at least one radiopaque object relative to the three-dimensional model and the position and orientation of the at least one feature associated with the at least one radiopaque object relative to the at least one endoscopic image; and
display at least a portion of the three-dimensional model based on the alignment of the three-dimensional model relative to the at least one endoscopic image.
